(19) United States
(12) Patent Application Publication
(10) Pub. No.: US 2023/0312652 A1
(43) Pub. Date: Oct. 5, 2023

(54) NOVEL PEPTIDES AND USES THEREOF

(71) Applicant: Aarhus Universitet, Aarhus C (DK)

(72) Inventors: Simon Mølgaard Jensen, Aarhus C (DK); Simon Glerup Pedersen, Aarhus C (DK); Anders Dalby, Aarhus C (DK)

(21) Appl. No.: 18/040,084

(22) PCT Filed: Aug. 6, 2021
(86) PCT No.: PCT/EP2021/071974

§ 371 Date: Jan. 31, 2023

(30) Foreign Application Priority Data
Aug. 6, 2020 (EP) 20189831.9

(51) Int. Cl. C07K 7/64; A61P 25/28; A61P 25/02; C07K 7/06

(52) U.S. Cl. CPC C07K 7/64; A61P 25/28; A61P 25/02; C07K 7/06; A61K 38/00

(57) ABSTRACT

The present invention concerns novel cyclic peptides, and medical uses thereof, such as treatment and/or prevention of diseases of the nervous system, neuropathic pain, and/or mental and behavioural disorders, and related aspects.

### TECHNICAL FIELD

The present invention concerns novel cyclic peptides, and medical uses thereof, such as treatment and/or prevention of diseases of the nervous system, neuropathic pain, and/or mental and behavioural disorders.

### BACKGROUND

Neurodegenerative diseases designate illnesses in which progressive loss of neuronal functions and synapses leading to apoptosis occurs in distinct brain areas. These include Alzheimer's disease, Parkinson's disease, Huntington's disease, amyotrophic lateral sclerosis (ALS) and frontotemporal dementia (FTD) among others. Hallmarks of neurodegenerative diseases include lack in neurotrophic signaling and aggregation of misfolded proteins, and the diseases are often linked with jammed neurotrophic-signaling caused by the aggregates.

In a healthy neuron, a variety of signalling pathways, initiated by neurotrophic growth factors, converge on the activation of transcription factor CREB leading to growth, neuronal plasticity and survival^{1,2}. In line with this, decreased activation of downstream transcription factor CREB is observed in Huntington's, Alzheimer's and FTD³⁻⁵.

Interestingly, distinct mutations linked to neurodegenerative diseases attenuate general clearing-mechanisms of misfolded proteins and damaged organelles in cells⁶. These include the lysosomal network, the proteasome-system and chaperone-mediated autophagy. For example, in Huntington's Disease, mutations of extensive CAG-repeats in exon 1 in the HTT gene cause the protein huntingtin to aggregate intranuclearly, which disrupts the autolysosomal network and reduce axonal transport of autophagosomes^{7,8}. Similarly, heterozygous loss of function mutations in the GRN gene has been linked to FTLD, in which mutations result in lysosomal dysfunction, which leads to aggregation of the protein TDP-43^{9,10}.

Thus, strategies for treating neurodegenerative diseases may include increasing activation of CREB and increasing clearance of misfolded proteins aggregates.

Recently, receptors of the Vps10p-domain receptor family, called SorCS1, SorCS2 and SorCS3, have emerged within neuroscience as they have shown to be deeply involved with neuronal viability and function¹¹⁻¹⁷. These receptors mediate the sorting and trafficking of a variety of ligands and receptors, which are crucial to neuronal viability and metabolic functions. Large cohort studies have highlighted the clinical relevance of SorCS1-3, linking them to several neurodegenerative and psychiatric disorders including Alzheimer's disease (AD), amyotrophic lateral sclerosis (ALS), Huntington's disease (HD), frontotemporal dementia (FTD), depression, schizophrenia, and ADHD¹⁸⁻²². Additionally, SorCS2 has been functionally linked with the severe neurological proteinopathies of amyotrophic lateral sclerosis (ALS) and HD^{23,24}. In these, SorCS2 has been shown to mis-localize to disease-aggregates resulting in its deficiency and acceleration of disease progression.

SorCS2 was further shown by Glerup et al. to be critical in mediating the signalling by brain-derived neurotrophic factor—a neurotrophin, which initiates survival and synaptic plasticity through activation of CREB 15. Interestingly, this mediation by SorCS2 was restricted to its intracellular domain. To a similar extent has the cytoplasmic domains of SorCS1 and 3-receptors previously been associated with their functions^{11, 25, 26}.

WO 2017/101956 relates to linear peptides and methods for modulating the phosphorylation of the Vps10 domain-containing receptor SorCS2, SorCS1 or SorCS3, and/or expression thereof. By said modulation, neoplastic disorders and disorders of the nervous system may be treated.

However, as of today, there is no treatment that can cure neurodegenerative diseases. While the number of patients with neurodegenerative disorders is growing rapidly as humans' life expectancy is increasing, there is a need for compounds that can be used to treat these diseases.

There remains a need for novel agents of use in the therapy or prophylaxis of diseases of the nervous system; neuropathic pain; mental and behavioural disorders; stroke; and metabolic disorders. Such agents may demonstrate: high potency; selectivity; improved safety profile; improved manufacturability; and/or desirable pharmacokinetic parameters, for example high brain availability and/or low clearance rate that reduces the dose and/or dose frequency required.

### SUMMARY

As outlined above, one promising strategy in treating neurodegenerative diseases is targeting the specific pathways mediated by the SorCS1-3 receptors through their cytoplasmic domain. The present inventors have thus developed seven novel peptides derived from the C-terminal cytoplasmic domain of the Vps10p domain receptors SorCS1, SorCS2 and SorCS3. In a main aspect, the present invention relates to a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7). Such peptides may be referred to herein as ‘peptides of the invention’.

Also provided is a cyclic peptide consisting of an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

In one aspect, the present invention relates to a cyclic peptide comprising an amino acid sequence selected from the group consisting of SEQ ID NO: 1 to 7 for use as a medicament.

In one aspect, the present invention relates to a cyclic peptide comprising an amino acid sequence selected from the group consisting of SEQ ID NO: 1 to 7 for use in the treatment and/or prevention of a disease or disorder selected from the group consisting of diseases of the nervous system; neuropathic pain; mental and behavioural disorders; stroke; and metabolic disorders.

It will be appreciated that peptides of the invention may form salts under appropriate conditions, therefore salts of peptides of the invention are also provided by the present invention, in particular pharmaceutically acceptable salts of the peptides of the invention. The peptides and their salts may exist in dissociated form in appropriate solvents, such as water.

### DESCRIPTION OF DRAWINGS

FIG. 1. Purification and Qualitative Check of Peptides P1, P2, P3, P4, P6, P8 and P9

HPLC chromatogram for Peptide P1 (SEQ ID NO: 1) with UV detection at 220 nm (A), LCMS chromatogram (B) and full scan acquisition positive ion mode spectrum (C). HPLC chromatogram for Peptide P2 (SEQ ID NO: 2) with UV detection at 215 nm (D) and MS full scan acquisition in positive ion mode (E). HPLC chromatogram for Peptide P3 (SEQ ID NO: 3) with UV detection at 215 nm (F) and MS full scan acquisition in positive ion mode (G). HPLC chromatogram for Peptide P4 (SEQ ID NO: 4) with UV detection at 215 nm (H) and MS full scan acquisition in positive ion mode (I). HPLC chromatogram for Peptide P6 (SEQ ID NO: 6) with UV detection at 215 nm (J) and MS full scan acquisition in positive ion mode (K). HPLC chromatogram for Peptide P8 (SEQ ID NO: 8) with UV detection at 215 nm (L) and MS full scan acquisition in positive ion mode (M). HPLC chromatogram for Peptide P9 (SEQ ID NO: 9) with UV detection at 215 nm (N) and MS full scan acquisition in positive ion mode (O).

FIG. 2. Peptides P1, P2 and P6 Increase Activation of CREB in Primary Neurons

Peptides P1 (A), P2 (B) and P6 (C) (SEQ ID NOs: 1, 2 and 6) activate CREB (phosphorylation on S133) in mouse primary cortical neurons compared to neurons stimulated with a scrambled peptide (Scr). Means±SEM. *p<0.05.

FIG. 3. Comparison of Peptide P1 and a Linear Analog (LP1) in the Activation of CREB in Wild-Type Cortical Neurons

Peptide P1 (SEQ ID NO: 1) and its corresponding linear peptide LP1 (SEQ ID NO: 8) with N- and C-terminal amidation and acetylation, respectively, both increased the activation of CREB (phosphorylation on S133) compared to neurons treated with scrambled peptide (Scr), however LP1 to a lesser extent. Means±SEM. *p<0.05.

FIG. 4. Peptides P1, P2, P4 and P6 Increase Survival in Wild-Type Cortical Neurons

Mouse cortical neurons were treated on day 7, 9 and 11 with 1 uM of peptides P2, P4 or P6 (SEQ ID NO: 2, 4 and 6). Living cells were subsequently assessed by MTT assay on the twelfth day in vitro (DIV12, the day of initiation being DIV0). The peptides increased the relative survival compared to neurons treated with a scrambled peptide (Scr). (A, B and C) Peptide P1 (SEQ ID NO: 1) survival assay using a serial of drug-dose concentration. Peptide P1 increased survival with an EC50 value of 2.7 pM. Means±SEM. *p<0.05 n=6. (D) FIG. 5. Downstream Effects of CREB Activation

1 uM of peptide P1 (SEQ ID NO: 1) significantly increases the levels of well-known downstream targets of CREB: neurotrophic factor BDNF (A), mitochondrial master regulator PGC1a (B) and lysosomal master regulator TFEB (C) in mouse primary neurons. These pathways are likewise activated in Huntington's Disease (HD) patient-derived fibroblasts (D, E and F respectively), thereby showing target engagement in cells with HD. Means±SEM. *p<0.05, **p<0.01. n=8

FIG. 6. Activation of Lysosomal Pathways

1 uM of peptide P1 (SEQ ID NO: 1) activates the lysosomal regulator AMPK by its phosphorylation on Threonine 172 (A). Inactivation of the direct-downstream target of AMPK, Raptor, was validated by its phosphorylation on Serine 792 (B), which was significantly more phosphorylated at this site following stimulation. This suggests that P1 activates pathways involved with lysosomal function. Means±SEM. *p<0.05, **p<0.01. n=8

FIG. 7. P1 Activation of AMPK and CREB is Blocked by STO-609 (CaMKK2-Inhibitor)

Mouse cortical neurons were pre-treated with 5 uM of the CaMKK2-inhibitor STO-609 for 1 hour followed stimulation with 1 uM of P1 (SEQ ID NO: 1). Activation of the lysosomal regulator AMPK and CREB was validated by their phosphorylation on Threonine 172 and Serine 133. Peptide P1 significantly increased phosphorylation of AMPK (A) and CREB (B), which was blocked by CaMKK2-inhibition, suggesting CaMKK2 is necessary for these activations. Means±SEM. *p<0.05. **p<0.01, ***p<0.001, ****p<0.0001. n=9.

FIG. 8. Peptide P1 Increases Lysosomal Acidification

Mouse wild-type hippocampal neurons treated with 1 uM of peptide P1 (SEQ ID NO: 1) show increased lysosomal acidification (as a measure of lysosomal activity) when measured using LysoSensor probe DND-189 in a live imaging setup. More than 1000 lysosomes were analysed per treatment, and the relative lysosomal intensity is displayed. After both 4 and 8 hours of treatment lysosomal acidification was increased in the peptide P1 treated cells, compared to neurons treated with a scrambled peptide (Scr), however after 24 hours no difference was observed. (A) LysoSensor DND-160 probe was used in a separate study to measure lysosomal acidification in SH-SY5Y cells (human neuroblastoma cell line) treated with peptide P1. Treatment of SH-SY5Y cells with P1 decreases the 340/380 nm ratio (B) which corresponds to a ˜0.2 drop in lysosomal pH (C). Means±SEM. *p<0.05. n=12 for (B) and (C)

FIG. 9. P1 Clears Soluble Mutated HTT in Huntington's Patient-Derived Fibroblasts (GM04719)

Peptide P1 (SEQ ID NO: 1) significantly reduces total HTT levels in Huntington's patient-derived fibroblasts (GM04719) after 8 hours of treatment. (A) In fibroblasts derived from a healthy individual (GM01650E) peptide P1 (SEQ ID NO: 1) increases HTT levels after 2-4 hours of stimulation, while no reduction at any timepoint is observed. (B) Treating HD fibroblasts once daily for 3 days with P1, significantly reduces the soluble levels of mutated HTT (measured using MW1 antibody specific for polyglutamine stretch). (C) No significant decrease in total HTT levels were observed (D). This indicates a therapeutic potential in HD. Means±SEM. *p<0.05. n=12

FIG. 10. P1 Increases Active Mitochondrial Mass in a Cell Model of HD (ST HDH)

Mitochondrial mass was measured in ST HDH cells (mouse striatal cell line) expressing either HTT with a 111 polyglutamine stretch (Q111) or a 7 polyglutamine stretch (Q7) when stimulated with P1 (SEQ ID NO: 1). The baseline mitochondrial mass in Q111 cells is significantly lower than the healthy cell (Q7), however when treated with P1 for 24 h the mitochondrial mass significantly increases above the baseline of Q7. P1 likewise induced mitochondrial biogenesis in the healthy cell line after 6 hours of stimulation. This demonstrates a potential rescue effect on mitochondrial function by P1 in Huntington's cells. Means±SEM. *p<0.05. n=12.

FIG. 11. P1 Reaches Brain by Both Subcutaneous and Intravenous Injection

Peptide P1 (SEQ ID NO: 1) was either subcutaneously or intravenously injected in wild-type mice in either 4.38 mM L-His, 140 mM NaCl, 0.2% Tween-20 and 1500 IU hyaluronidase (for SC, pH 6.15) or saline (IV). At 13 mg/kg plasma (A) and whole brain (B) concentrations and at 52 mg/kg plasma, whole brain and cerebrospinal fluid concentrations were validated at different timepoints by LC MS/MS. Half-life (T½) for P1 in brain following IV delivery was 0.232 hours, while SC delivery showed T½ of 0.316 hours. The maximal brain-plasma ratio was 0.032 for SC delivery and 0.034 for IV delivery, thereby showing that P1 reaches the brain following IV and SC delivery. Means±SEM. n=3

FIG. 12. P1 Dose-Dependent Delivery to the Brain Following SC Injection

Peptide P1 (SEQ ID NO: 1) was subcutaneously injected in wild-type mice in different concentrations of 0 to 52 mg/kg in 4.38 mM L-His, 140 mM NaCl, 0.2% Tween-20 and 1500 IU hyaluronidase (pH 6.15). Levels of P1 were validated in both plasma (A), whole brain (B) and cerebrospinal fluid (C) 15 min after injection by LC MS/MS. P1 is measurable in the brain and CSF at all concentrations. Means±SEM. n=3.

FIG. 13. Delivery of P1 to the Brain by SC Injection in Different Formulations

13 mg/kg of peptide P1 (SEQ ID NO: 1) was subcutaneously injected in wild-type mice in different formulations in either PBS buffer, in buffer containing 4.38 mM L-His, 140 mM NaCl, 0.2% Tween-20 and 1500 IU hyaluronidase (pH 6.15) or in buffer with 4.38 mM L-His, 140 mM NaCl, 0.2% Tween-20. Levels of P1 were validated in both plasma (A) and whole brain (B) 15 and 30 min. after injection by LC MS/MS. P1 is measurable in the plasma and brain in all formulations while no significant difference is observed between L-His buffer with or without the enzyme hyaluronidase, demonstrating that brain delivery of P1 does not require hyaluronidase. Means±SEM. *p<0.05. n=3.

FIG. 14. Peptide P1 Displays CREB-Activation in Striatum and Hippocampus of Wild-Type Mice Following IV Injection

Wild-type mice were injected with 0.26 mg/kg of peptide P1 (SEQ ID NO: 1), its linear analog LP1 (SEQ ID NO: 8) or a scrambled peptide (Scr) intravenously dissolved in isotonic saline. 1 hour after injection, pCREB (phosphorylation on S133) levels in the striatum (A) and hippocampus (B) were assessed. Both LP1 and P1 increase phosphorylated CREB. Means±SEM. *p<0.05.

FIG. 15. Subcutaneous Injection of Peptide P1 Activates CREB and AMPK in Striatum of Wild-Type Mice

Wild-type mice were injected with 0-26 mg/kg of peptide P1 (SEQ ID NO: 1) subcutaneously in 4.38 mM L-His, 140 mM NaCl, 0.2% Tween-20 and 1500 IU hyaluronidase (pH 6.15). 2 hours after injection, pCREB (phosphorylation on S133) and pAMPK levels in the striatum were assessed by western blotting normalized to beta-actin levels. As shown, peptide P1 significantly activated transcription factor CREB (A) at doses between 0.13-26 mg/kg and AMPK (B) at doses between 0.13-13 mg/kg in the striatum in wild-type mice (n=10). In a separate study, a linear version of P1 attached to a cell-penetrating TAT moiety (P9), was tested for its ability to activate CREB in both striatum and hippocampus following subcutaneous injection of 3.6 mg/kg using the same formulation (C). pCREB levels following administration of P9 were not increased significantly in either hippocampus or striatum tissue. Means±SEM. *p<0.05.

FIG. 16. P1 Pathway Engagement in Striatum of Wild-Type Mice

Wild-type mice were injected with 13 mg/kg of P1 (SEQ ID NO: 1) subcutaneously in 4.38 mM L-His, 140 mM NaCl, 0.2% Tween-20 and 1500 IU hyaluronidase (pH 6.15). The mice were sacrificed at timepoints between 2-8 hours after injection. Levels of pCREB, TFEB, downstream lysosomal gene products LAMP1, p62/SQSTM1, PGRN and mitochondrial master regulator PGC1a were validated by western blotting. All proteins were normalized to beta-actin levels. As shown, P1 significantly activates CREB after 2 hours (A), although no significant effect is seen at 4 hours. TFEB (B) and LAMP1 (C) are significantly increased at 2 and 4 hours, while both PGRN (E) and PGC1a (F) are significantly increased at 4 hours. Furthermore, autophagic flux was validated through assessing p62 levels (D). P62 levels decline after 4 hours and are significantly lowered at 8 hours following injection, demonstrating increased lysosomal activation in striatum of the wild-type mice. Means±SEM. *p<0.05. n=15-20.

FIG. 17. Daily Subcutaneous Administration of P1 Increases Pro-Survival and Mitochondrial Proteins in Cortex of R6/2 Mice

R6/2 mice were subcutaneously injected with a daily dose of 13 mg/kg of P1 (SEQ ID NO: 1) between 8 weeks to 12 weeks of age (late stage in disease-development). Levels of DARPP32 (marker of medium spiny neurons, MSN), mature BDNF (mBDNF), TrkB full-length and PGC1a in cortex were validated. Despite, the late start of treatment relative to disease-development, mice treated with P1 demonstrate higher basal levels of both DARPP32 (A) and mature BDNF (B). In addition, both TrkB FL levels and PGC1a levels are largely restored. Means±SEM. *p<0.05, **p<0.01, ***p<0.001. n=12.

FIG. 18. Daily Subcutaneous Administration of P1 Increases Brain Weight and Activity Dependent Behaviour in R6/2 Mice

R6/2 mice were subcutaneously injected with a daily dose of 13 mg/kg of P1 (SEQ ID NO: 1) between 8 weeks to 12 weeks of age (late stage in disease development). At 12 weeks of age, mice were sacrificed and their brains removed and weighed. P1-treated mice increased brain weight by an average of 14 mg. (A) At 11 weeks of age, mice were subjected to an open field behaviour test. Distance travelled and number of rears were recorded over a 20 min. period. P1 treated mice show significantly higher distance travelled (B) and rearing frequency (C) compared to vehicle treated controls. Throughout treatment, the mice motor functions were measured by clasping (D) and rotarod (E). Initiating treatment of R6/2 mice with P1 in a late-stage of disease (between week 8-12) shows a tendency to improve motor functions in both clasping and rotarod. Means±SEM. *p<0.05. n=12.

FIG. 19. Daily Subcutaneous Administration of P1 Shows Tendency to Increase Cortex and Hippocampal Volume in R6/2 Mice

R6/2 mice were subcutaneously injected with a daily dose of 13 mg/kg of P1 (SEQ ID NO: 1) between 8 weeks to 12 weeks of age (late stage in disease development). At 12 weeks of age, mice were sacrificed and their brains removed and regional brain volume was measured by stereological counting in both cortex (A), hippocampus (B) and striatum (C). P1 shows a trend to increase neuronal survival in both cortex and hippocampus, despite initiating treatment at late stage in disease progression. Means±SEM. n=12.

FIG. 20. P1 is Stable in Plasma and Brain Homogenates

Mouse or human plasma were incubated with 2 μM P1 (SEQ ID NO: 1) or propantheline bromide (positive control for degradation). At different timepoints supernatant was analysed by LCMS. P1 shows limited degradation in either mouse or human plasma with a half-life of more than 289 minutes (A and C). Plasma binding was likewise measured in which P1 shows very low plasma binding in mouse plasma while 20% plasma binding in human plasma (B and D). Likewise, mouse brain homogenate was incubated with 2 μM P1 (SEQ ID NO: 1) with or without protease inhibitors or 7-Ethoxycoumarin (positive control of degradation). At the outlined time points, enzymatic reactions were stopped and samples analysed by LCMS. T½ of P1 was 245 minutes in brain homogenate (E). For brain homogenate binding, 2 μM P1 or propranolol (positive control) was used. P1 showed less than 20% brain homogenate binding (F).

FIG. 21. P1 Metabolic Stability in Liver S9 Fractions and Liver Microsomes

Liver S9 fractions from 5 different species were incubated with 2 μM P1 (SEQ ID NO: 1) or 7-Ethoxycoumarin (positive control of clearance). Samples were analysed by LCMS at indicated timepoints. P1 shows low clearance and high stability in liver S9 fractions (A). Likewise, liver microsomes from mouse and human were incubated with 2 μM P1 (SEQ ID NO: 1) or Diclofenac (positive control for degradation) and necessary reactants for up to 60 minutes. Samples were analysed by LCMS for mouse (C) and human (D). P1 shows low clearance and high stability in liver microsomes.

FIG. 22. P1 Stability in L-His+Tween 20+Hyaluronidase pH 6.15

P1 (SEQ ID NO: 1) stability in buffer solution containing 4.4 mM L-histidine, 140 mM NaCl, 0.2% w/V Tween 20 and 1500 IU/mL hyaluronidase was investigated by incubation at −20° C., 4° C. and 25° C. for up to 7 days. LCMS was used to ascertain the amount of P1 remaining in the solutions. Data show no notable losses at −20° C. and 4° C.

FIG. 23. Dose-Dependent Effect of P1 on Cytochrome P450 Activity

Liver microsomes were prepared with cocktails of known substrates of the respective CYP450 enzymes to evaluate the dose-range effects of P1 (SEQ ID NO: 1) on enzyme inhibition. Positive controls were used for each enzyme at a single dose. The data shows no CYP-inhibition effects of P1 on the validated enzymes (A to G), neither with nor without NADPH.

FIG. 24. P1 Effect on HERG Potassium Channels Using Patch-Clamp

The effect of P1 (SEQ ID NO: 1) on hERG potassium channels (a surrogate for IKr, the rapidly activating, delayed rectifier cardiac potassium current) was evaluated at doses between 0.1-30 μM. No inhibition was observed at the tested concentrations.

FIG. 25. Peptide P1 Increases Clearance of Cytoplasmic TDP-43 in HEK293 Cells

HEK293 cells were transfected with TDP-43 ANLS (TDP-43 lacking nuclear localisation signal leading to aberrant cytoplasmic accumulation). The following day the cells were stimulated with the indicated doses of peptide P1 (SEQ ID NO: 1) for 24 hours. TDP-43 levels were validated by western blotting. Peptide P1 decreased the pathogenic cytoplasmic form of TDP-43, indicating a therapeutic potential in frontotemporal dementia.

FIG. 26. Peptide P1 Increases Neuronal Branching in GRN-Deficient Neurons

Hippocampal neurons from GRN+/− mice were stimulated at DIV1 with 1 uM peptide P1 (SEQ ID NO: 1). At DIV4 the neurons were fixed and stained for MAP2. The number of branches were subsequently counted per neuron. Treating neurons suffering from frontotemporal dementia, increases their branching, which demonstrates a neurotrophic activity of peptide P1 in this this model of frontotemporal dementia (GRN+/−). Means±SEM. *p<0.05. n=12.

FIG. 27. Peptide P1 Activates Transcription Factor CREB in GRN-Deficient Neurons

Cortical neurons from GRN+/− mice were stimulated for 10 minutes with either vehicle or peptide P1 (SEQ ID NO: 1) (1 uM). Phosphorylation of CREB (S133) was assessed and validated by western blotting. As shown, peptide P1 is able to activate CREB in an in vitro model of frontotemporal dementia (GRN+/−). Means±SEM. *p<0.05.

FIG. 28. Peptides P2, P4 and P6 Increase Survival in GRN-Deficient Cortical Neurons

(A-C) GRN(+/−) cortical neurons were treated on day 7, 9, 11, 13 and 15 with 1 uM of peptides P2, P4 or P6 (SEQ ID NOs: 2, 4 and 6). Living cells were subsequently assessed by MTT assay on DIV16. The peptides increased the relative survival compared to neurons treated with a scrambled peptide (Scr). Means±SEM. *p<0.05.

FIG. 29. P1 Increases Lysosomal Acidification GRN+/− Hippocampal Neurons

Five days in vitro (DIV) GRN+/− hippocampal neurons were treated with 1 uM of peptide P1 (SEQ ID NO: 1) for 4 hours and subsequently assessed by live-imaging atthe indicated timepoints using LysoSensor probe DND-189. The relative lysosomal intensity is displayed, as a measure of lysosomal acidification. Peptide P1 increases lysosomal acidification, compared to neurons treated with a scrambled peptide (Scr). Means±SEM. *p<0.05, **p<0.01, ***p<0.001, ****p<0.0001.

FIG. 30. P1 Increases GRN in Wild-Type Mice Following 7-Day Daily SC Injections

Wild-type mice were injected with a daily dose of 13 mg/kg of P1 (SEQ ID NO: 1) subcutaneously in 4.38 mM L-His, 140 mM NaCl, 0.2% Tween-20 and 1500 IU hyaluronidase (pH 6.15) for 7 days. The mice were sacrificed on day 8. Levels of GRN in the hippocampus were determined. As shown, SC administration of P1 increases basal GRN levels compared to vehicle-treated in the hippocampus. Means±SEM. *p<0.05, **p<0.01. n=5.

FIG. 31. Peptide P3 Acutely Attenuates Neuropathic Pain in a Spared Nerve Injury Mouse Model

Subcutaneous administration of peptide P3 (SEQ ID NO: 3) in 4.38 mM L-His, 140 mM NaCl and 0.2% maltoside, in a spared nerve injury mouse model (the tibial nerve is crushed to induce neuropathic pain) attenuates the pain in an acute manner measured by von frey test, for up to 2% hours. Means±SEM. *p<0.05.

FIG. 32. Peptide P3 Ameliorates Chronic Neuropathic Pain in a Spared Nerve Injury Mouse Model

Daily subcutaneous administration for 7 days with peptide P3 (SEQ ID NO: 3) in 4.38 mM L-His, 140 mM NaCl and 0.2% maltoside, in a spared nerve injury mouse model, ameliorates the chronic neuropathic pain measured by von frey test. Means±SEM. *p<0.05. n=7.

FIG. 33. P6 Increases Neuronal Branching in Wild-Type Neurons

Hippocampal neurons from wild-type mice were stimulated at DIV1 with 0.1 uM or 1 uM P6 (SEQ ID NO: 6). BDNF was used as positive control. At DIV4 the neurons were fixed and stained for MAP2. The number of branches were subsequently counted per neuron. Treating neurons with P6 increases their branching, which demonstrates a neurotrophic activity of P6. Means±SEM. *p<0.05. n=12.

FIG. 34. P6 Increases SV2A in Wild-Type Cortical Neurons

Primary neurons (DIV8) were treated with 1 uM of P6 (SEQ ID NO: 6). At DIV11 the neurons were lysed and expression of SV2A, marker of synaptic vesicles, were validated by western blotting. P6-treated neurons display increased SV2A levels. Means±SEM. *p<0.05, **p<0.01. n=25.

FIG. 35 Peptide P9

### DESCRIPTION OF SEQUENCES

SEQ ID NO: 1 Cyclic peptide P1
SEQ ID NO: 2 Cyclic peptide P2
SEQ ID NO: 3 Cyclic peptide P3
SEQ ID NO: 4 Cyclic peptide P4
SEQ ID NO: 5 Cyclic peptide P5
SEQ ID NO: 6 Cyclic peptide P6
SEQ ID NO: 7 Cyclic peptide P7
SEQ ID NO: 8 Linear N-amidated and C-acetylated peptide LP1
SEQ ID NO: 9 Linear TAT-sequence containing peptide P9
SEQ ID NO: 10 Linear peptide P1A
SEQ ID NO: 11 Linear peptide P2A
SEQ ID NO: 12 Linear peptide P3A
SEQ ID NO: 13 Linear peptide P4A
SEQ ID NO: 14 Linear peptide P5A
SEQ ID NO: 15 Linear peptide P6A
SEQ ID NO: 16 Linear peptide P7A
SEQ ID NO: 17 Native SorCS2 peptide
SEQ ID NO: 18 Native SorCS1 peptide
SEQ ID NO: 19 Native SorCS3 peptide
SEQ ID NO: 20 Linear peptide P1B
SEQ ID NO: 21 Linear peptide P1C
SEQ ID NO: 22 Linear peptide P1D
SEQ ID NO: 23 Linear peptide P1E
SEQ ID NO: 24 Linear peptide P1F
SEQ ID NO: 25 Linear peptide P1G
SEQ ID NO: 26 Linear peptide P1H
SEQ ID NO: 27 Linear peptide P11
SEQ ID NO: 28 Linear peptide P1J
SEQ ID NO: 29 Linear peptide P1K
SEQ ID NO: 30 Linear peptide P2B
SEQ ID NO: 31 Linear peptide P2C
SEQ ID NO: 32 Linear peptide P2D
SEQ ID NO: 33 Linear peptide P2E
SEQ ID NO: 34 Linear peptide P2F
SEQ ID NO: 35 Linear peptide P2G
SEQ ID NO: 36 Linear peptide P2H
SEQ ID NO: 37 Linear peptide P21
SEQ ID NO: 38 Linear peptide P2J
SEQ ID NO: 39 Linear peptide P2K
SEQ ID NO: 40 Linear peptide P3B
SEQ ID NO: 41 Linear peptide P3C
SEQ ID NO: 42 Linear peptide P3D
SEQ ID NO: 43 Linear peptide P3E
SEQ ID NO: 44 Linear peptide P3F
SEQ ID NO: 45 Linear peptide P3G
SEQ ID NO: 46 Linear peptide P3H
SEQ ID NO: 47 Linear peptide P31
SEQ ID NO: 48 Linear peptide P3J
SEQ ID NO: 49 Linear peptide P3K
SEQ ID NO: 50 Linear peptide P4B
SEQ ID NO: 51 Linear peptide P4C
SEQ ID NO: 52 Linear peptide P4D
SEQ ID NO: 53 Linear peptide P4E
SEQ ID NO: 54 Linear peptide P4F
SEQ ID NO: 55 Linear peptide P4G
SEQ ID NO: 56 Linear peptide P4H
SEQ ID NO: 57 Linear peptide P41
SEQ ID NO: 58 Linear peptide P4J
SEQ ID NO: 59 Linear peptide P4K
SEQ ID NO: 60 Linear peptide P5B
SEQ ID NO: 61 Linear peptide P5C
SEQ ID NO: 62 Linear peptide P5D
SEQ ID NO: 63 Linear peptide P5E
SEQ ID NO: 64 Linear peptide P5F
SEQ ID NO: 65 Linear peptide P5G
SEQ ID NO: 66 Linear peptide P5H
SEQ ID NO: 67 Linear peptide P51
SEQ ID NO: 68 Linear peptide P5J
SEQ ID NO: 69 Linear peptide P5K
SEQ ID NO: 70 Linear peptide P6B
SEQ ID NO: 71 Linear peptide P6C
SEQ ID NO: 72 Linear peptide P6D
SEQ ID NO: 73 Linear peptide P6E
SEQ ID NO: 74 Linear peptide P6F
SEQ ID NO: 75 Linear peptide P6G
SEQ ID NO: 76 Linear peptide P6H
SEQ ID NO: 77 Linear peptide P61
SEQ ID NO: 78 Linear peptide P7J
SEQ ID NO: 79 Linear peptide P7K
SEQ ID NO: 80 Linear peptide P7B
SEQ ID NO: 81 Linear peptide P7C
SEQ ID NO: 82 Linear peptide P7D
SEQ ID NO: 83 Linear peptide P7E
SEQ ID NO: 84 Linear peptide P7F
SEQ ID NO: 85 Linear peptide P7G
SEQ ID NO: 86 Linear peptide P7H
SEQ ID NO: 87 Linear peptide P71
SEQ ID NO: 88 Linear peptide P7J
SEQ ID NO: 89 Linear peptide P7K
SEQ ID NO: 90 Scrambled peptide connected to cell-penetrating moiety (TAT-sequence)

### DETAILED DESCRIPTION

### Definition of Abbreviations and Terms

“CREB” (CREB-TF, cAMP response element-binding protein) is a cellular transcription factor, which binds to certain DNA sequences called cAMP response elements (CRE), thereby increasing or decreasing the transcription of the genes. Genes whose transcription is regulated by CREB include: c-fos, BDNF, tyrosine hydroxylase, numerous neuropeptides (such as somatostatin, enkephalin, VGF, corticotropin-releasing hormone) and genes involved in the mammalian circadian clock (PER1, PER2). CREB has a well-documented role in neuronal plasticity and long-term memory formation in the brain and has been shown to be integral in the formation of spatial memory.

A “peptide” or “protein” is a polymer of amino acid residues preferably joined exclusively by peptide bonds, whether produced naturally or synthetically. Said proteins or peptides may or may not have been post-translationally modified. A peptide is usually shorter in length than a protein, and single-chained. In some embodiments, the peptides may be modified, such as modified after preparation, such as post-translationally. In other embodiments the peptides are not modified, such as modified after preparation.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications.

In some embodiments, the peptides of the invention are intended for prophylactic use, i.e. administration to a subject to prevent or reduce the risk of developing a condition, disease or disorder. “Preventing” or “prevention” refers to hindering the development of a condition, disease or disorder, and includes the administration of a peptide of the invention to prevent or reduce the risk of the onset of symptoms or complications.

In some embodiments, the peptides of the invention are intended for therapeutic use, i.e. administration to a subject having a condition, disease or disorder. The therapeutic use may be intended to alleviate or relieve symptoms or complications; delay the progression of the condition, disease or disorder; cure or eliminate the condition, disease or disorder.

A “subject in need thereof” refers to an individual who may benefit from the present invention. In one embodiment, said subject in need thereof is an individual suffering from diseases of the nervous system, neuropathic pain, and/or mental and behavioural disorders. The subject to be treated is preferably a mammal, in particular a human being. Treatment of animals, such as mice, rats, dogs, cats, cows, horses, sheep and pigs, is, however, also within the scope of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with peptides of the invention.

Peptides

In one aspect, the present invention relates to a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

| SEQ ID NO | Sequence | |
|---|---|---|
| 1 | [MTEPVEHEEDV] | Cyclic (backbone) |
| 2 | [MTDPVDHDEDV] | Cyclic (backbone) |
| 3 | [MTAPVAHAEDV] | Cyclic (backbone) |
| 4 | [MIEPVEHEESR] | Cyclic (backbone) |
| 5 | [MIDPVDHDESR] | Cyclic (backbone) |
| 6 | [MIGSVEQEENA] | Cyclic (backbone) |
| 7 | [MIGSVDQDENA] | Cyclic (backbone) |

The difference between the peptide of SEQ ID NO: 1 and the peptide of SEQ ID NO: 2 is that the glutamic acid residues (E) at positions 3, 6 and 8 are replaced with aspartic acid residues (D).

The difference between the peptide of SEQ ID NO: 4 and the peptide of SEQ ID NO: 5 is that the glutamic acid residues (E) at positions 3, 6 and 8 are replaced with aspartic acid residues (D).

The difference between the peptide of SEQ ID NO: 6 and the peptide of SEQ ID NO: 7 is that the glutamic acid residues (E) at positions 6 and 8 are replaced with aspartic acid residues (D).

In some embodiments, the present invention provides a cyclic peptide consisting of an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

In one aspect, the present invention provides a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a salt thereof, in particular a pharmaceutically acceptable salt.

In some embodiments, the present invention provides a cyclic peptide consisting of an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a salt thereof, in particular a pharmaceutically acceptable salt.

In one aspect, the present invention provides a salt, in particular a pharmaceutically acceptable salt, of a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

In some embodiments, the present invention provides a salt, in particular a pharmaceutically acceptable salt, of a cyclic peptide consisting of an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

In some embodiments, the cyclic peptide consists of the amino acid sequence of MTEPVEHEEDV (SEQ ID NO: 1). In some embodiments, the cyclic peptide consists of the amino acid sequence of MTEPVEHEEDV (SEQ ID NO: 1), or a salt thereof, in particular a pharmaceutically acceptable salt.

In some embodiments, the cyclic peptide consists of the amino acid sequence of MTDPVDHDEDV (SEQ ID NO: 2). In some embodiments, the cyclic peptide consists of the amino acid sequence of MTDPVDHDEDV (SEQ ID NO: 2), or a salt thereof, in particular a pharmaceutically acceptable salt.

In some embodiments, the cyclic peptide consists of the amino acid sequence of MTAPVAHAEDV (SEQ ID NO: 3). In some embodiments, the cyclic peptide consists of the amino acid sequence of MTAPVAHAEDV (SEQ ID NO: 3), or a salt thereof, in particular a pharmaceutically acceptable salt.

In some embodiments, the cyclic peptide consists of the amino acid sequence of MIEPVEHEESR (SEQ ID NO: 4). In some embodiments, the cyclic peptide consists of the amino acid sequence of MIEPVEHEESR (SEQ ID NO: 4), or a salt thereof, in particular a pharmaceutically acceptable salt.

In some embodiments, the cyclic peptide consists of the amino acid sequence of MIDPVDHDESR (SEQ ID NO: 5). In some embodiments, the cyclic peptide consists of the amino acid sequence of MIDPVDHDESR (SEQ ID NO: 5), or a salt thereof, in particular a pharmaceutically acceptable salt.

In some embodiments, the cyclic peptide consists of the amino acid sequence of MIGSVEQEENA (SEQ ID NO: 6). In some embodiments, the cyclic peptide consists of the amino acid sequence of MIGSVEQEENA (SEQ ID NO: 6), or a salt thereof, in particular a pharmaceutically acceptable salt.

In some embodiments, the cyclic peptide consists of the amino acid sequence of MIGSVDQDENA (SEQ ID NO: 7). In some embodiments, the cyclic peptide consists of the amino acid sequence of MIGSVDQDENA (SEQ ID NO: 7), or a salt thereof, in particular a pharmaceutically acceptable salt.

The peptides of the present invention are cyclic. A peptide can typically be cyclized in four different ways: side chain-to-side chain, tail-to-side chain, side chain-to-head and head-to-tail. As used herein, the term “head-to-tail cyclized peptide” is used interchangeably with the term “backbone cyclized peptide”. In one embodiment, the cyclic peptide is a backbone cyclized peptide. In one embodiment, the cyclic peptide is formed by the formation of an amide bond between its N-terminus- and its C-terminus-parts, i.e. head-to tail cyclization.

In some embodiments the peptide is cyclized side chain-to-side chain and the backbone of the peptide is joined exclusively by peptide bonds.

In some embodiments the peptide is cyclized tail-to-side chain and the backbone of the peptide is joined exclusively by peptide bonds.

In some embodiments the peptide is cyclized side chain-to-head and the backbone of the peptide is joined exclusively by peptide bonds.

In some embodiments the peptide is backbone cyclized and the backbone of the peptide is joined exclusively by peptide bonds. In some embodiments the peptide is backbone cyclized and wherein all residues of the peptide are joined exclusively by peptide bonds. Hence, in one embodiment, the cyclic peptide consists of eleven amide-bonded amino acid residues of the sequence selected from SEQ ID NO: 1 to 7.

In one embodiment, the cyclic peptides comprise no more than 50 amino acid residues, such as no more than 40 amino acid residues, such as no more than 30 amino acid residues, such as no more than 20 amino acid residues. Desirably, the cyclic peptides comprise no more than 14 amino acid residues, such as no more than 13 amino acid residues, such as no more than 12 amino acid residues. Accordingly, the cyclic peptides of the invention are at least 11 amino acid residues, such as 11 amino acid residues.

In one embodiment, the cyclic peptide consists of no more than 20 amino acid residues, such as no more than 15 amino acid residues, such as no more than 14 amino acid residues, such as no more than 13 amino acid residues, such as no more than 12 amino acid residues. Accordingly, the peptides of the invention are at least 11 amino acid residues.

In one embodiment, the cyclic peptide consists of an amino acid sequence of no more than 20 amino acid residues, wherein the peptide is backbone cyclized and all residues are connected via peptide bonds and comprising the sequence MTEPVEHEEDV (SEQ ID NO: 1). In one embodiment, the cyclic peptide consists of the amino acid sequence MTEPVEHEEDV (SEQ ID NO: 1), wherein the peptide is backbone cyclized and all residues are connected via peptide bonds.

In one embodiment, the cyclic peptide consists of an amino acid sequence of MTEPVEHEEDV (SEQ ID NO: 1), wherein the peptide is backbone cyclized via the M amino acid residue in position 1 and the V amino acid residue in position 11.

In one embodiment, the cyclic peptide consists of an amino acid sequence of no more than 20 amino acid residues, wherein the peptide is backbone cyclized and all residues are connected via peptide bonds and comprising the sequence MTDPVDHDEDV (SEQ ID NO: 2). In one embodiment, the cyclic peptide consists of the amino acid sequence MTDPVDHDEDV (SEQ ID NO: 2), wherein the peptide is backbone cyclized and all residues are connected via peptide bonds.

In one embodiment, the cyclic peptide consists of an amino acid sequence of MTDPVDHDEDV (SEQ ID NO: 2), wherein the peptide is backbone cyclized via the M amino acid residue in position 1 and the V amino acid residue in position 11.

In one embodiment, the cyclic peptide consists of an amino acid sequence of no more than 20 amino acid residues, wherein the peptide is backbone cyclized and all residues are connected via peptide bonds and comprising the sequence MTAPVAHAEDV (SEQ ID NO: 3). In one embodiment, the cyclic peptide consists of the amino acid sequence MTAPVAHAEDV (SEQ ID NO: 3), wherein the peptide is backbone cyclized and all residues are connected via peptide bonds.

In one embodiment, the peptide consists of an amino acid sequence of MTAPVAHAEDV (SEQ ID NO: 3), wherein the peptide is backbone cyclized via the M amino acid residue in position 1 and the V amino acid residue in position 11.

In one embodiment, the cyclic peptide consists of an amino acid sequence of no more than 20 amino acid residues, wherein the peptide is backbone cyclized and all residues are connected via peptide bonds and comprising the sequence MIEPVEHEESR (SEQ ID NO: 4). In one embodiment, the cyclic peptide consists of the amino acid sequence MIEPVEHEESR (SEQ ID NO: 4), wherein the peptide is backbone cyclized and all residues are connected via peptide bonds.

In one embodiment, the peptide consists of an amino acid sequence of MIEPVEHEESR (SEQ ID NO: 4), wherein the peptide is backbone cyclized via the M amino acid residue in position 1 and the R amino acid residue in position 11.

In one embodiment, the cyclic peptide consists of an amino acid sequence of no more than 20 amino acid residues, wherein the peptide is backbone cyclized and all residues are connected via peptide bonds and comprising the sequence MIDPVDHDESR (SEQ ID NO: 5). In one embodiment, the cyclic peptide consists of the amino acid sequence MIDPVDHDESR (SEQ ID NO: 5), wherein the peptide is backbone cyclized and all residues are connected via peptide bonds.

In one embodiment, the peptide consists of an amino acid sequence of MIDPVDHDESR (SEQ ID NO: 5), wherein the peptide is backbone cyclized via the M amino acid residue in position 1 and the R amino acid residue in position 11.

In one embodiment, the cyclic peptide consists of an amino acid sequence of no more than 20 amino acid residues, wherein the peptide is backbone cyclized and all residues are connected via peptide bonds and comprising the sequence MIGSVEQEENA (SEQ ID NO: 6). In one embodiment, the cyclic peptide consists of the amino acid sequence MIGSVEQEENA (SEQ ID NO: 6), wherein the peptide is backbone cyclized and all residues are connected via peptide bonds.

In one embodiment, the peptide consists of an amino acid sequence of MIGSVEQEENA (SEQ ID NO: 6), wherein the peptide is backbone cyclized via the M amino acid residue in position 1 and the A amino acid residue in position 11.

In one embodiment, the cyclic peptide consists of an amino acid sequence of no more than 20 amino acid residues, wherein the peptide is backbone cyclized and all residues are connected via peptide bonds and comprising the sequence MIGSVDQDENA (SEQ ID NO: 7). In one embodiment, the cyclic peptide consists of the amino acid sequence MIGSVDQDENA (SEQ ID NO: 7), wherein the peptide is backbone cyclized and all residues are connected via peptide bonds.

In one embodiment, the peptide consists of an amino acid sequence of MIGSVDQDENA (SEQ ID NO: 7), wherein the peptide is backbone cyclized via the M amino acid residue in position 1 and the A amino acid residue in position 11.

In one embodiment, the peptide is further conjugated to a detectable moiety.

Polynucleotides, Vectors and Cells

In one aspect, the present invention concerns a polynucleotide encoding the corresponding linear sequence of the cyclic peptide as defined herein. In one embodiment, said corresponding linear sequence of the cyclic peptide as defined herein comprises an amino acid sequence selected from the group consisting of SEQ ID NO: 10 to 16 and 20 to 89. In one aspect, the present invention concerns a vector comprising said polynucleotide. In one aspect, the present invention concerns a host cell comprising said polynucleotide or said vector. In one embodiment, the host cell is a bacterial cell. In one embodiment, the host cell is a mammalian cell. In one embodiment, the host cell is a human cell. In one embodiment, the host cell is an isolated mammalian cell. In one embodiment, the host cell is an isolated human cell.

In one aspect of the present invention there is provided a nucleic acid construct encoding for a peptide comprising an amino acid sequence selected from the group consisting of SEQ ID NO: 10 to 16 and 20 to 89. By nucleic acid construct is understood a genetically engineered nucleic acid. The nucleic acid construct may be a non-replicating and linear nucleic acid, a circular expression vector or an autonomously replicating plasmid. The nucleic acid construct may be replicating or non-replicating. The nucleic acid construct may be linear or circular. The nucleic acid construct may be DNA or RNA. The nucleic acid construct may be codon optimised for expression in a particular host cell. The nucleic acid construct may contain naturally occurring or modified residues, suitably only naturally occurring residues.

Group 1

| | | |
|---|---|---|
| SEQ ID NO: 10 | Linear peptide P1A | MTEPVEHEEDV |
| SEQ ID NO: 20 | Linear peptide P1B | VMTEPVEHEED |
| SEQ ID NO: 21 | Linear peptide P1C | DVMTEPVEHEE |
| SEQ ID NO: 22 | Linear peptide P1D | EDVMTEPVEHE |
| SEQ ID NO: 23 | Linear peptide P1E | EEDVMTEPVEH |
| SEQ ID NO: 24 | Linear peptide P1F | HEEDVMTEPVE |
| SEQ ID NO: 25 | Linear peptide P1G | EHEEDVMTEPV |
| SEQ ID NO: 26 | Linear peptide P1H | VEHEEDVMTEP |
| SEQ ID NO: 27 | Linear peptide P1I | PVEHEEDVMTE |
| SEQ ID NO: 28 | Linear peptide P1J | EPVEHEEDVMT |
| SEQ ID NO: 29 | Linear peptide P1K | TEPVEHEEDVM |

Group 2

| | | |
|---|---|---|
| SEQ ID NO: 11 | Linear peptide P2A | MTDPVDHDEDV |
| SEQ ID NO: 30 | Linear peptide P2B | VMTDPVDHDED |
| SEQ ID NO: 31 | Linear peptide P2C | DVMTDPVDHDE |
| SEQ ID NO: 32 | Linear peptide P2D | EDVMTDPVDHD |
| SEQ ID NO: 33 | Linear peptide P2E | DEDVMTDPVDH |
| SEQ ID NO: 34 | Linear peptide P2F | HDEDVMTDPVD |
| SEQ ID NO: 35 | Linear peptide P2G | DHDEDVMTDPV |
| SEQ ID NO: 36 | Linear peptide P2H | VDHDEDVMTDP |
| SEQ ID NO: 37 | Linear peptide P2I | PVDHDEDVMTD |
| SEQ ID NO: 38 | Linear peptide P2J | DPVDHDEDVMT |
| SEQ ID NO: 39 | Linear peptide P2K | TDPVDHDEDVM |

Group 3

| | | |
|---|---|---|
| SEQ ID NO: 12 | Linear peptide P3A | MTAPVAHAEDV |
| SEQ ID NO: 40 | Linear peptide P3B | VMTAPVAHAED |
| SEQ ID NO: 41 | Linear peptide P3C | DVMTAPVAHAE |
| SEQ ID NO: 42 | Linear peptide P3D | EDVMTAPVAHA |
| SEQ ID NO: 43 | Linear peptide P3E | AEDVMTAPVAH |
| SEQ ID NO: 44 | Linear peptide P3F | HAEDVMTAPVA |
| SEQ ID NO: 45 | Linear peptide P3G | AHAEDVMTAPV |
| SEQ ID NO: 46 | Linear peptide P3H | VAHAEDVMTAP |
| SEQ ID NO: 47 | Linear peptide P3I | PVAHAEDVMTA |
| SEQ ID NO: 48 | Linear peptide P3J | APVAHAEDVMT |
| SEQ ID NO: 49 | Linear peptide P3K | TAPVAHAEDVM |

Group 4

| | | |
|---|---|---|
| SEQ ID NO: 13 | Linear peptide P4A | MIEPVEHEESR |
| SEQ ID NO: 50 | Linear peptide P4B | RMIEPVEHEES |
| SEQ ID NO: 51 | Linear peptide P4C | SRMIEPVEHEE |
| SEQ ID NO: 52 | Linear peptide P4D | ESRMIEPVEHE |
| SEQ ID NO: 53 | Linear peptide P4E | EESRMIEPVEH |
| SEQ ID NO: 54 | Linear peptide P4F | HEESRMIEPVE |
| SEQ ID NO: 55 | Linear peptide P4G | EHEESRMIEPV |
| SEQ ID NO: 56 | Linear peptide P4H | VEHEESRMIEP |
| SEQ ID NO: 57 | Linear peptide P4I | PVEHEESRMIE |
| SEQ ID NO: 58 | Linear peptide P4J | EPVEHEESRMI |
| SEQ ID NO: 59 | Linear peptide P4K | IEPVEHEESRM |

Group 5

| | | |
|---|---|---|
| SEQ ID NO: 14 | Linear peptide P5A | MIDPVDHDESR |
| SEQ ID NO: 60 | Linear peptide P5B | RMIDPVDHDES |
| SEQ ID NO: 61 | Linear peptide P5C | SRMIDPVDHDE |
| SEQ ID NO: 62 | Linear peptide P5D | ESRMIDPVDHD |
| SEQ ID NO: 63 | Linear peptide P5E | DESRMIDPVDH |
| SEQ ID NO: 64 | Linear peptide P5F | HDESRMIDPVD |
| SEQ ID NO: 65 | Linear peptide P5G | DHDESRMIDPV |
| SEQ ID NO: 66 | Linear peptide P5H | VDHDESRMIDP |
| SEQ ID NO: 67 | Linear peptide P5I | PVDHDESRMID |
| SEQ ID NO: 68 | Linear peptide P5J | DPVDHDESRMI |
| SEQ ID NO: 69 | Linear peptide P5K | IDPVDHDESRM |

Group 6

| | | |
|---|---|---|
| SEQ ID NO: 15 | Linear peptide P6A | MIGSVEQEENA |
| SEQ ID NO: 70 | Linear peptide P6B | AMIGSVEQEEN |
| SEQ ID NO: 71 | Linear peptide P6C | NAMIGSVEQEE |
| SEQ ID NO: 72 | Linear peptide P6D | ENAMIGSVEQE |
| SEQ ID NO: 73 | Linear peptide P6E | EENAMIGSVEQ |
| SEQ ID NO: 74 | Linear peptide P6F | QEENAMIGSVE |
| SEQ ID NO: 75 | Linear peptide P6G | EQEENAMIGSV |
| SEQ ID NO: 76 | Linear peptide P6H | VEQEENAMIGS |
| SEQ ID NO: 77 | Linear peptide P6I | SVEQEENAMIG |
| SEQ ID NO: 78 | Linear peptide P6J | GSVEQEENAMI |
| SEQ ID NO: 79 | Linear peptide P6K | IGSVEQEENAM |

Group 7

| | | |
|---|---|---|
| SEQ ID NO: 16 | Linear peptide P7A | MIGSVDQDENA |
| SEQ ID NO: 80 | Linear peptide P7B | AMIGSVDQDEN |
| SEQ ID NO: 81 | Linear peptide P7C | NAMIGSVDQDE |
| SEQ ID NO: 82 | Linear peptide P7D | ENAMIGSVDQD |
| SEQ ID NO: 83 | Linear peptide P7E | DENAMIGSVDQ |
| SEQ ID NO: 84 | Linear peptide P7F | QDENAMIGSVD |
| SEQ ID NO: 85 | Linear peptide P7G | DQDENAMIGSV |
| SEQ ID NO: 86 | Linear peptide P7H | VDQDENAMIGS |
| SEQ ID NO: 87 | Linear peptide P7I | SVDQDENAMIG |
| SEQ ID NO: 88 | Linear peptide P7J | GSVDQDENAMI |
| SEQ ID NO: 89 | Linear peptide P7K | IGSVDQDENAM |

In one embodiment the nucleic acid construct encodes for and is capable of expressing a peptide comprising an amino acid sequence selected from Group 1. In one embodiment the nucleic acid construct encodes for and is capable of expressing a peptide comprising an amino acid sequence selected from Group 2. In one embodiment the nucleic acid construct encodes for and is capable of expressing a peptide comprising an amino acid sequence selected from Group 3. In one embodiment the nucleic acid construct encodes for and is capable of expressing a peptide comprising an amino acid sequence selected from Group 4. In one embodiment the nucleic acid construct encodes for and is capable of expressing a peptide comprising an amino acid sequence selected from Group 5. In one embodiment the nucleic acid construct encodes for and is capable of expressing a peptide comprising an amino acid sequence selected from Group 6. In one embodiment the nucleic acid construct encodes for and is capable of expressing a peptide comprising an amino acid sequence selected from Group 7.

Suitably the encoded peptides comprise no more than 50 amino acid residues, such as no more than 40 amino acid residues, such as no more than 30 amino acid residues, such as no more than 20 amino acid residues. Desirably, the cyclic peptides comprise no more than 14 amino acid residues, such as no more than 13 amino acid residues, such as no more than 12 amino acid residues. Accordingly, the cyclic peptides of the invention are at least 11 amino acid residues, such as 11 amino acid residues.

A nucleic acid construct encoding for and being capable of expressing a peptide consisting of an amino acid sequence selected from the group consisting of SEQ ID NO: 10 to 16 and 20 to 89 is also provided.

Method for Preparation of Peptides

The peptides according to the present invention may be prepared by any methods known in the art. Thus, the peptides of SEQ ID NOs: 1 to 7 may be prepared by standard peptide-preparation techniques, such as solution synthesis or Merrifield-type solid phase synthesis.

In one embodiment, a peptide according to the invention is synthetically made or produced. The methods for synthetic production of peptides are well known in the art. Detailed descriptions as well as practical advice for producing synthetic peptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999. In one embodiment, the peptide or peptide sequences of the invention are produced synthetically, in particular, by the Sequence Assisted Peptide Synthesis (SAPS) method, by solution synthesis, by Solid-phase peptide synthesis (SPPS) such as Merrifield-type solid phase synthesis, by recombinant techniques (production by host cells comprising a first nucleic acid sequence encoding the peptide operably associated with a second nucleic acid capable of directing expression in said host cells) or enzymatic synthesis. These are well-known to the skilled person.

After purification of the linear peptides, such as by reversed phase HPLC, the linear peptides are further processed to cyclic peptides. Techniques for cyclizing a peptide and for obtaining a cyclic peptide, for example by using a solid support, are well known by the man skilled in the art.

In one aspect, the present invention concerns a method of manufacturing a cyclic peptide of the invention, the method comprising the steps of:
(i) preparing a linear peptide having an appropriate amino acid sequence, and
(ii) subsequently generating a cyclized peptide from the linear peptide.

An appropriate amino acid sequence is one which when cyclised provides a cyclic peptide comprising or consisting of an amino acid sequence selected from the group consisting of SEQ ID NOs: 1 to 7. A side chain cyclised, head to side chain or tail to side chain cyclised peptide requires a linear sequence in normal N to C-terminal residue order. However, a backbone cyclized peptide consisting of P1 may be formed from a linear peptide MTEPVEHEEDV, VEHEEDVMTEP or the like.

The linear peptide will typically be joined exclusively by peptide bonds. The cyclized peptide may be backbone cyclized. The cyclized peptide will typically be joined exclusively by peptide bonds.

Synthetic preparation of a linear peptide may require or benefit from the presence of side chain protecting groups on some or all residues containing side chains which may be reactive, and side chain protecting groups may or may not be removed, or may be removed and reintroduced, depending on the particular sequence, prior to generation of a cyclized peptide, such as a backbone cyclised peptide. If some side chain protection is present during generation of a cyclized peptide, such as a backbone cyclised peptide, this may subsequently be removed to form a deprotected cyclised peptide. In preparation of a non-backbone cyclised peptide, protecting groups may be present at the N- or C-termini as required.

The linear peptide and/or the cyclized peptide (or protected versions thereof as appropriate) may be in the form of a salt, in particular a pharmaceutically acceptable salt.

The present invention provides a peptide comprising a linear amino acid sequence selected from the group consisting of SEQ ID NO: 10 to 16 and 20 to 89, or a protected version thereof, such as a side chain protected version thereof. The present invention provides a linear peptide consisting of an amino acid sequence selected from the group consisting of SEQ ID NO: 10 to 16 and 20 to 89, or a protected version thereof, such as a side chain protected version thereof.

In one embodiment, the linear peptide comprises an amino acid sequence selected from Group 1, or a protected version thereof, such as a side chain protected version thereof. In one embodiment the linear peptide comprises an amino acid sequence selected from Group 2, or a protected version thereof, such as a side chain protected version thereof. In one embodiment the linear peptide comprises an amino acid sequence selected from Group 3, or a protected version thereof, such as a side chain protected version thereof. In one embodiment the linear peptide comprises an amino acid sequence selected from Group 4, or a protected version thereof, such as a side chain protected version thereof. In one embodiment the linear peptide comprises an amino acid sequence selected from Group 5, or a protected version thereof, such as a side chain protected version thereof. In one embodiment the linear peptide comprises an amino acid sequence selected from Group 6, or a protected version thereof, such as a side chain protected version thereof. In one embodiment the linear peptide comprises an amino acid sequence selected from Group 7, or a protected version thereof, such as a side chain protected version thereof. The linear peptide, or protected version thereof, may be in the form of a salt, in particular a pharmaceutically acceptable salt.

The present invention provides a side chain protected version of a cyclic peptide comprising an amino acid sequence selected from the group consisting of SEQ ID NO: 1 to 7. The present invention provides a side chain protected version of a cyclic peptide consisting of an amino acid sequence selected from the group consisting of SEQ ID NO: 1 to 7. The protected version of a cyclic peptide, may be in the form of a salt, in particular a pharmaceutically acceptable salt.

Amino acid protecting groups are known to the skilled person and are discussed, for example, in Isidro-Llobet et al, Chem Rev 2009 109 2455-2504 and Chandrudu et al, Molecules 2013 18(4):4373-4388. Common side chain protections include: Arg(Pbf), Asn(Trt), Asp(OtBu), Cys(Trt), Gln(Trt), Glu(OtBu), His(Trt), Lys(Boc), Ser(tBu), Thr(tBu) and Tyr(tBu).

In one aspect, the present invention concerns a method of manufacturing a cyclic peptide as defined herein, the method comprising the steps of preparing a linear peptide comprising or consisting of an amino acid sequence selected from the group consisting of SEQ ID NOs: 1 to 7, and subsequently generating a backbone cyclized peptide of the linear peptide. In one embodiment, the linear peptide is prepared by recombinantly expressing the peptide, for example in an E. coli system. In one embodiment, the linear peptide is prepared synthetically.

Thus, in one aspect, the present invention relates to a peptide comprising or consisting of an amino acid sequence selected from the group consisting of MTEPVEHEEDV (SEQ ID NO: 10), MTDPVDHDEDV (SEQ ID NO: 11), MTAPVAHAEDV (SEQ ID NO: 12), MIEPVEHEESR (SEQ ID NO: 13), MIDPVDHDESR (SEQ ID NO: 14), MIGSVEQEENA (SEQ ID NO: 15) and MIGSVDQDENA (SEQ ID NO: 16).

Biological Activity

As demonstrated in the examples disclosed herein, the peptides of the present invention can promote neuronal survival.

Neurodegenerative diseases are often linked with jammed neurotrophic-signaling caused by the aggregates of misfolded proteins. In a healthy neuron, a variety of signaling pathways, initiated by neurotrophic growth factors, converge on the activation of transcription factor CREB leading to growth, neuronal plasticity and survival. However, decreased activation of downstream transcription factor CREB is observed in a number of neurodegenerative diseases. As demonstrated in Example 3, cyclic peptides comprising an amino acid sequence of SEQ ID NO: 1, 2 or 6 activate CREB. Thus, in one embodiment, a cyclic peptide consisting of an amino acid sequence of MTEPVEHEEDV (SEQ ID NO: 1), MTDPVDHDEDV (SEQ ID NO: 2) or MIGSVEQEENA (SEQ ID NO: 6), is capable of increasing CREB activity.

Further, as demonstrated for cyclic peptides of SEQ ID NOs: 1, 2, 4 and 6 in Example 5, peptides of the present invention can increase the relative survival of cortical neurons.

A hallmark of neurodegenerative diseases is aggregation of misfolded proteins. Mutations linked to neurodegenerative diseases have been shown to attenuate general clearing-mechanisms of misfolded proteins and damaged organelles in cells. Importantly, as demonstrated in Example 9, the cyclic peptide of SEQ ID NO: 1 is able to increase lysosomal acidification, and consequently also increase the clearance of toxic aggregates. Further, a cyclic peptide of SEQ ID NO: 1 increases clearance of soluble mutated huntingtin, which aggregates in Huntington's disease (Example 10), as well as clearance of cytoplasmic protein TDP-43 (Example 22), which aggregates in frontotemporal dementia and ALS.

In one aspect, the present invention concerns a method of increasing the number of synapses, said method comprising administration of the peptide as defined herein to a subject in need thereof.

The impact of administration of peptides of the invention may be quantified in various ways. For example, in the context of Huntington's the Unified Huntington's Disease Rating Scale (UHDRS) can be applied as a measure of motor function, cognition, behavior abnormalities and functional capacity, which may be improved. Other Huntington markers include measuring mutated huntingtin in cerebrospinal fluid (CSF) of a subject, which may be reduced.

Total functional capacity score (TFC) may be improved.

CSF or blood plasma levels of neurofilament light-chain (nf-L), a prognostic marker of cognitive decline, may be reduced.

Magnetic Resonance Imaging (MRI) may be used to quantity brain volume, either entire brain or specific regions, which may be increased.

Medical Use

In one aspect, the present invention provides a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof, for use as a medicament.

In one aspect, the present invention provides a cyclic peptide consisting of an amino acid sequence selected from the group consisting of SEQ ID NO: 1 to 7, or a pharmaceutically acceptable salt thereof, for use as a medicament.

In one aspect, the present invention relates to a cyclic peptide consisting of SEQ ID NO: 1 to 7 for use as a medicament.

In one aspect, the present invention relates to cyclic peptide of SEQ ID NO: 1 to 7 for use in the treatment and/or prevention of a disease or disorder selected from the group consisting of diseases of the nervous system; neuropathic pain; mental and behavioural disorders; stroke and metabolic disorders.

In one aspect, the present invention concerns a method of treatment or prevention of a disease or disorder selected from the group consisting of diseases of the nervous system; neuropathic pain; mental and behavioural disorders; stroke and metabolic disorders, said method comprising administering the cyclic peptide as defined herein to a subject in need thereof.

In one aspect, the present invention relates to the use of the cyclic peptide as defined herein for the manufacture of a medicament for the treatment and/or prevention of a disease or disorder selected from the group consisting of diseases of the nervous system; neuropathic pain; mental and behavioural disorders; stroke and metabolic disorders.

In one aspect, the present invention provides a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof, for use in the therapy of a disease of the nervous system; neuropathic pain; a mental or behavioural disorder; stroke; or a metabolic disorder.

In one aspect, the present invention provides a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof, for use in the prophylaxis of a disease of the nervous system; neuropathic pain; a mental or behavioural disorder; stroke; or a metabolic disorder.

In one aspect, the present invention provides a method of therapy of a disease of the nervous system; neuropathic pain; a mental or behavioural disorder; stroke; or a metabolic disorder, said method comprising administering to a subject a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof.

In one aspect, the present invention provides a method of prophylaxis of a disease of the nervous system; neuropathic pain; a mental or behavioural disorder; stroke or a metabolic disorder, said method comprising administering to a subject a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof.

In one aspect, the present invention provides the use of a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof, for the manufacture of a medicament.

In one aspect, the present invention provides the use of a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof, for the manufacture of a medicament for the therapy of a disease of the nervous system; neuropathic pain; a mental or behavioural disorder; stroke or a metabolic disorder.

In one aspect, the present invention provides the use of a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof, for the manufacture of a medicament for the prophylaxis of a disease of the nervous system; neuropathic pain; a mental or behavioural disorder; stroke or a metabolic disorder.

Suitably the peptide of the invention, or a pharmaceutically acceptable salt thereof, is administered to a subject in need thereof. Suitably the peptide of the invention, or a pharmaceutically acceptable salt thereof, is administered in a safe and effective amount i.e. an amount providing an acceptable balance of desired benefits and undesired side effects. A “safe and effective amount” is intended to include an amount that is effective to achieve a desirable effect in therapy and/or prophylaxis. A desirable effect is typically clinically significant and/or measurable, for instance in the context of (a) preventing a condition, disease or disorder occurring, in particular, when a subject is predisposed or at risk but has not yet been diagnosed; (b) inhibiting a condition, disease or disorder, i.e., slowing or arresting its development; and/or (c) relieving a condition, disease or disorder, i.e., causing regression of the condition, disease or disorder or a reduction in associated symptoms. The safe and effective amount may be one that is sufficient to achieve the desirable effect either when the peptide of the invention, or a pharmaceutically acceptable salt thereof, is administered alone or alternatively when it is administered in combination with one or more further active pharmaceutical ingredients, which either are further peptides of the invention, or a pharmaceutically acceptable salts thereof, or are different from the peptides of the invention.

The cyclic peptide of SEQ ID NO: 1 promotes neuronal survival, lysosomal acidification and removal of toxic aggregates demonstrated in Example 5, Example 9 and Example 10. The cyclic peptide of SEQ ID NO: 1 is derived from SorCS2, which has been described as a regulator of BDNF-signaling, important for the development of depression^{15,24,27}. In one embodiment, the cyclic peptide of SEQ ID NO: 1 is for use in the treatment or prevention of Huntington's disease, amyotrophic lateral sclerosis (ALS), Parkinson's disease, Alzheimer's disease, Frontotemporal dementia (FTD), depression and/or stroke.

In one embodiment, the cyclic peptide of SEQ ID NO: 1, or a pharmaceutically acceptable salt thereof, is for use in the treatment or prevention of Huntington's disease, amyotrophic lateral sclerosis (ALS), Parkinson's disease, Alzheimer's disease, Frontotemporal dementia (FTD), depression and/or stroke.

In one embodiment, the cyclic peptide of SEQ ID NO: 1, or a pharmaceutically acceptable salt thereof, is for the therapy of Huntington's disease. In one embodiment, the cyclic peptide of SEQ ID NO: 1, or a pharmaceutically acceptable salt thereof, is for the prophylaxis of Huntington's disease.

In one embodiment, the cyclic peptide of SEQ ID NO: 1, or a pharmaceutically acceptable salt thereof, is for the therapy of frontotemporal dementia. In one embodiment, the cyclic peptide of SEQ ID NO: 1, or a pharmaceutically acceptable salt thereof, is for the prophylaxis of frontotemporal dementia.

In one embodiment, the cyclic peptide of SEQ ID NO: 1, or a pharmaceutically acceptable salt thereof, is for the therapy of Parkinson's disease. In one embodiment, the cyclic peptide of SEQ ID NO: 1, or a pharmaceutically acceptable salt thereof, is for the prophylaxis of Parkinson's disease (including A, B and C).

In one embodiment, the cyclic peptide of SEQ ID NO: 1, or a pharmaceutically acceptable salt thereof, is for the therapy of lysosomal storage disorders, such as Nieman-Pick disease. In one embodiment, the cyclic peptide of SEQ ID NO: 1, or a pharmaceutically acceptable salt thereof, is for the prophylaxis of lysosomal storage disorders, such as Nieman-Pick disease (including A, B and C).

In one embodiment, the cyclic peptide of SEQ ID NO: 1, or a pharmaceutically acceptable salt thereof, is for the therapy of WAGR syndrome. In one embodiment, the cyclic peptide of SEQ ID NO: 1, or a pharmaceutically acceptable salt thereof, is for the prophylaxis of WAGR syndrome.

In one embodiment, the cyclic peptide of SEQ ID NO: 1, or a pharmaceutically acceptable salt thereof, is for the therapy of dementia. In one embodiment, the cyclic peptide of SEQ ID NO: 1, or a pharmaceutically acceptable salt thereof, is for the prophylaxis of dementia.

The cyclic peptide of SEQ ID NO: 2 promotes neuronal survival demonstrated in Example 5. The cyclic peptide of SEQ ID NO: 2 is derived from SorCS2, which has been described as a regulator of BDNF-signaling, important for the development of depression. In one embodiment, the cyclic peptide of SEQ ID NO: 2 is for use in the treatment or prevention of Huntington's disease, amyotrophic lateral sclerosis (ALS), Parkinson's disease, Alzheimer's disease, Frontotemporal dementia (FTD), depression and/or stroke.

In one embodiment, the cyclic peptide of SEQ ID NO: 2, or a pharmaceutically acceptable salt thereof, is for use in the treatment or prevention of Huntington's disease, amyotrophic lateral sclerosis (ALS), Parkinson's disease, Alzheimer's disease, Frontotemporal dementia (FTD), depression and/or stroke.

In one embodiment, the cyclic peptide of SEQ ID NO: 2, or a pharmaceutically acceptable salt thereof, is for the therapy of Huntington's disease. In one embodiment, the cyclic peptide of SEQ ID NO: 2, or a pharmaceutically acceptable salt thereof, is for the prophylaxis of Huntington's disease.

In one embodiment, the cyclic peptide of SEQ ID NO: 2, or a pharmaceutically acceptable salt thereof, is for the therapy of frontotemporal dementia. In one embodiment, the cyclic peptide of SEQ ID NO: 2, or a pharmaceutically acceptable salt thereof, is for the prophylaxis of frontotemporal dementia.

In one embodiment, the cyclic peptide of SEQ ID NO: 2, or a pharmaceutically acceptable salt thereof, is for the therapy of Parkinson's disease. In one embodiment, the cyclic peptide of SEQ ID NO: 2, or a pharmaceutically acceptable salt thereof, is for the prophylaxis of Parkinson's disease.

In one embodiment, the cyclic peptide of SEQ ID NO: 2, or a pharmaceutically acceptable salt thereof, is for the therapy of lysosomal storage disorders, such as Nieman-Pick disease. In one embodiment, the cyclic peptide of SEQ ID NO: 2, or a pharmaceutically acceptable salt thereof, is for the prophylaxis of lysosomal storage disorders, such as Nieman-Pick disease.

In one embodiment, the cyclic peptide of SEQ ID NO: 2, or a pharmaceutically acceptable salt thereof, is for the therapy of WAGR syndrome, such as Nieman-Pick disease. In one embodiment, the cyclic peptide of SEQ ID NO: 2, or a pharmaceutically acceptable salt thereof, is for the prophylaxis of WAGR syndrome.

In one embodiment, the cyclic peptide of SEQ ID NO: 2, or a pharmaceutically acceptable salt thereof, is for the therapy of dementia. In one embodiment, the cyclic peptide of SEQ ID NO: 2, or a pharmaceutically acceptable salt thereof, is for the prophylaxis of dementia.

The cyclic peptide of SEQ ID NO: 3 ameliorates neuropathic pain in mice demonstrated in Example 28 and 29. The cyclic peptide of SEQ ID NO: 3 is derived from SorCS2, which has been shown to have a functional link to stroke and epilepsy^{27,28}. In one embodiment, the cyclic peptide of SEQ ID NO: 3 is for use in the treatment or prevention of neuropathic pain, stroke and/or epilepsy.

In one embodiment, the cyclic peptide of SEQ ID NO: 3, or a pharmaceutically acceptable salt thereof, is for use in the treatment or prevention of neuropathic pain, stroke and/or epilepsy.

The cyclic peptide of SEQ ID NO: 4 promotes neuronal survival demonstrated in Example 5. The cyclic peptide of SEQ ID NO: 4 is derived from SorCS1, which has a strong genetic link to metabolic diseases e.g., diabetes mellitus type 1, diabetes mellitus type 2, obesity and/or nonalcoholic fatty liver disease (NAFLD)²⁹⁻³³. In one embodiment, the cyclic peptide of SEQ ID NO: 4 is for use in the treatment or prevention of Huntington's disease, Parkinson's disease, Alzheimer's disease, Frontotemporal dementia (FTD), diabetes mellitus type 1, diabetes mellitus type 2, obesity and/or nonalcoholic fatty liver disease (NAFLD).

In one embodiment, the cyclic peptide of SEQ ID NO: 4, or a pharmaceutically acceptable salt thereof, is for use in the treatment or prevention of Huntington's disease, Parkinson's disease, Alzheimer's disease, Frontotemporal dementia (FTD), diabetes mellitus type 1, diabetes mellitus type 2, obesity and/or nonalcoholic fatty liver disease (NAFLD).

The cyclic peptide of SEQ ID NO: 5 is derived from SorCS1, which has a strong genetic link to metabolic diseases e.g., diabetes mellitus type 1, diabetes mellitus type 2, obesity and/or nonalcoholic fatty liver disease (NAFLD)²⁹⁻³³. In one embodiment, the cyclic peptide of SEQ ID NO: 5 is for use in the treatment or prevention of Huntington's disease, Parkinson's disease, Alzheimer's disease, Frontotemporal dementia (FTD), diabetes mellitus type 1, diabetes mellitus type 2, obesity and/or nonalcoholic fatty liver disease (NAFLD).

In one embodiment, the cyclic peptide of SEQ ID NO: 5, or a pharmaceutically acceptable salt thereof, is for use in the treatment or prevention of Huntington's disease, Parkinson's disease, Alzheimer's disease, Frontotemporal dementia (FTD), diabetes mellitus type 1, diabetes mellitus type 2, obesity and/or nonalcoholic fatty liver disease (NAFLD).

The cyclic peptide of SEQ ID NO: 6 promotes neuronal survival demonstrated in Example 5. The cyclic peptide of SEQ ID NO: 6 is derived from SorCS3, which has a strong genetic link to several psychiatric disorders as depression, anxiety, post-traumatic stress disorder (PTSD), Schizophrenia (SZ), attention deficit hyperactivity disorder (ADHD), autism and/or an autism related disorder such as selected from the group consisting of Rett syndrome, Fragile X syndrome and Angelman syndrome³⁴⁻³⁷. In one embodiment, the cyclic peptide of SEQ ID NO: 6 is for use in the treatment or prevention of Huntington's disease, Parkinson's disease, Alzheimer's disease, Frontotemporal dementia (FTD), depression, anxiety, post-traumatic stress disorder (PTSD), Schizophrenia (SZ), attention deficit hyperactivity disorder (ADHD), autism and/or an autism related disorder, such as selected from the group consisting of Rett syndrome, Fragile X syndrome and Angelman syndrome.

In one embodiment, the cyclic peptide of SEQ ID NO: 6, or a pharmaceutically acceptable salt thereof, is for use in the treatment or prevention of Huntington's disease, Parkinson's disease, Alzheimer's disease, Frontotemporal dementia (FTD), depression, anxiety, post-traumatic stress disorder (PTSD), Schizophrenia (SZ), attention deficit hyperactivity disorder (ADHD), autism and/or an autism related disorder, such as selected from the group consisting of Rett syndrome, Fragile X syndrome and Angelman syndrome.

SorCS3 has a strong genetic link to several psychiatric disorders as depression, anxiety, post-traumatic stress disorder (PTSD), Schizophrenia (SZ), attention deficit hyperactivity disorder (ADHD), autism and/or an autism related disorder such as selected from the group consisting of Rett syndrome, Fragile X syndrome and Angelman syndrome³⁴⁻³⁷. In one embodiment, the cyclic peptide of SEQ ID NO: 7 is for use in the treatment or prevention of Huntington's disease, Parkinson's disease, Alzheimer's disease, Frontotemporal dementia (FTD), depression, anxiety, post-traumatic stress disorder (PTSD), Schizophrenia (SZ), attention deficit hyperactivity disorder (ADHD), autism and/or an autism related disorder, such as selected from the group consisting of Rett syndrome, Fragile X syndrome and Angelman syndrome.

In one embodiment, the cyclic peptide of SEQ ID NO: 7, or a pharmaceutically acceptable salt thereof, is for use in the treatment or prevention of Huntington's disease, Parkinson's disease, Alzheimer's disease, Frontotemporal dementia (FTD), depression, anxiety, post-traumatic stress disorder (PTSD), Schizophrenia (SZ), attention deficit hyperactivity disorder (ADHD), autism and/or an autism related disorder, such as selected from the group consisting of Rett syndrome, Fragile X syndrome and Angelman syndrome.

Diseases of the Nervous System

In one embodiment, the present invention relates to a cyclic peptide as defined herein for use in the treatment and/or prevention of diseases of the nervous system.

In one embodiment, the diseases of the nervous system are selected from the group consisting of Huntington's disease, amyotrophic lateral sclerosis (ALS), Parkinson's disease, Alzheimer's disease, Frontotemporal dementia (FTD) and epilepsy. In one embodiment, the cyclic peptide of SEQ ID NO: 1 is for use in the treatment and/or prevention of a diseases of the nervous system, such as selected from the group consisting of Huntington's disease, Frontotemporal dementia (FTD), amyotrophic lateral sclerosis (ALS), Parkinson's disease and Alzheimer's disease. In one embodiment, the cyclic peptide of SEQ ID NO: 2 is for use in the treatment and/or prevention of a disease of the nervous system, such as selected from the group consisting of Huntington's disease, Frontotemporal dementia (FTD), amyotrophic lateral sclerosis (ALS), Parkinson's disease and Alzheimer's disease In one embodiment, the cyclic peptide of SEQ ID NO: 3 is for use in the treatment and/or prevention of a disease of the nervous system, such as epilepsy. In one embodiment, the cyclic peptide of SEQ ID NO: 4 is for use in the treatment and/or prevention of a disease of the nervous system, such as selected from the group consisting of Alzheimer's disease, Parkinson's disease, Frontotemporal dementia (FTD), and Huntington's disease. In one embodiment, the cyclic peptide of SEQ ID NO: 5 is for use in the treatment and/or prevention of a disease of the nervous system, such as selected from the group consisting of Alzheimer's disease, Parkinson's disease, Frontotemporal dementia (FTD), and Huntington's disease. In one embodiment, the cyclic peptide of SEQ ID NO: 6 is for use in the treatment and/or prevention of a disease of the nervous system, such as selected from the group consisting of Alzheimer's disease, Parkinson's disease, Frontotemporal dementia (FTD), and Huntington's disease. In one embodiment, the cyclic peptide of SEQ ID NO: 7 is for use in the treatment and/or prevention of a disease of the nervous system, such as selected from the group consisting of Alzheimer's disease, Parkinson's disease, Frontotemporal dementia (FTD), and Huntington's disease.

In one embodiment, the cyclic peptide of SEQ ID NO: 1, or a pharmaceutically acceptable salt thereof, is for use in the treatment and/or prevention of a diseases of the nervous system, such as selected from the group consisting of Huntington's disease, Frontotemporal dementia (FTD), amyotrophic lateral sclerosis (ALS), Parkinson's disease and Alzheimer's disease. In one embodiment, the cyclic peptide of SEQ ID NO: 2, or a pharmaceutically acceptable salt thereof, is for use in the treatment and/or prevention of a disease of the nervous system, such as selected from the group consisting of Huntington's disease, Frontotemporal dementia (FTD), amyotrophic lateral sclerosis (ALS), Parkinson's disease and Alzheimer's disease In one embodiment, the cyclic peptide of SEQ ID NO: 3, or a pharmaceutically acceptable salt thereof, is for use in the treatment and/or prevention of a disease of the nervous system, such as epilepsy. In one embodiment, the cyclic peptide of SEQ ID NO: 4, or a pharmaceutically acceptable salt thereof, is for use in the treatment and/or prevention of a disease of the nervous system, such as selected from the group consisting of Alzheimer's disease, Parkinson's disease, Frontotemporal dementia (FTD), and Huntington's disease. In one embodiment, the cyclic peptide of SEQ ID NO: 5, or a pharmaceutically acceptable salt thereof, is for use in the treatment and/or prevention of a disease of the nervous system, such as selected from the group consisting of Alzheimer's disease, Parkinson's disease, Frontotemporal dementia (FTD), and Huntington's disease. In one embodiment, the cyclic peptide of SEQ ID NO: 6, or a pharmaceutically acceptable salt thereof, is for use in the treatment and/or prevention of a disease of the nervous system, such as selected from the group consisting of Alzheimer's disease, Parkinson's disease, Frontotemporal dementia (FTD), and Huntington's disease. In one embodiment, the cyclic peptide of SEQ ID NO: 7, or a pharmaceutically acceptable salt thereof, is for use in the treatment and/or prevention of a disease of the nervous system, such as selected from the group consisting of Alzheimer's disease, Parkinson's disease, Frontotemporal dementia (FTD), and Huntington's disease.

In one embodiment, the disease of the nervous system is a neurodegenerative disease. Neurodegeneration is the progressive loss of structure or function of neurons, including death of neurons. Many neurodegenerative diseases, such as amyotrophic lateral sclerosis (ALS), Parkinson's disease, Alzheimer's disease and Huntington's disease, occur as a result of neurodegenerative processes. In one embodiment, the neurodegenerative disease is frontotemporal lobar dementia. In one embodiment, the neurodegenerative disease is Huntington's disease. In one embodiment, the neurodegenerative disease is Alzheimer's disease. In one embodiment, the neurodegenerative disease is Parkinson's disease. In one embodiment, the neurodegenerative disease is amyotrophic lateral sclerosis. As demonstrated in Example 9, treatment with a cyclic peptide of SEQ ID NO: 1 causes an increase in lysosomal acidification, which is one of the mechanisms that clear toxic aggregates present in neurodegenerative diseases. Hence, in one embodiment, the cyclic peptide, of SEQ ID NO: 1 is for use in the treatment and/or prevention of a neurodegenerative disease. In one embodiment, the cyclic peptide of SEQ ID NO: 2 is for use in the treatment and/or prevention of a neurodegenerative disease. In one embodiment, the cyclic peptide of SEQ ID NO: 4 is for use in the treatment and/or prevention of a neurodegenerative disease. In one embodiment, the cyclic peptide of SEQ ID NO: is for use in the treatment and/or prevention of a neurodegenerative disease. In one embodiment, the cyclic peptide of SEQ ID NO: 6 is for use in the treatment and/or prevention of a neurodegenerative disease. In one embodiment, the cyclic peptide of SEQ ID NO: 7 is for use in the treatment and/or prevention of a neurodegenerative disease.

In one embodiment, the cyclic peptide, of SEQ ID NO: 1, or a pharmaceutically acceptable salt thereof, is for use in the treatment and/or prevention of a neurodegenerative disease. In one embodiment, the cyclic peptide of SEQ ID NO: 2, or a pharmaceutically acceptable salt thereof, is for use in the treatment and/or prevention of a neurodegenerative disease. In one embodiment, the cyclic peptide of SEQ ID NO: 4, or a pharmaceutically acceptable salt thereof, is for use in the treatment and/or prevention of a neurodegenerative disease. In one embodiment, the cyclic peptide of SEQ ID NO: 5, or a pharmaceutically acceptable salt thereof, is for use in the treatment and/or prevention of a neurodegenerative disease. In one embodiment, the cyclic peptide of SEQ ID NO: 6, or a pharmaceutically acceptable salt thereof, is for use in the treatment and/or prevention of a neurodegenerative disease. In one embodiment, the cyclic peptide of SEQ ID NO: 7, or a pharmaceutically acceptable salt thereof, is for use in the treatment and/or prevention of a neurodegenerative disease.

Mental and Behavioural Disorders

The cyclic peptides are derived from SorCS1, SorCS2 and SorCS3. SorCS3 has a strong genetic link to several psychiatric disorders as depression, anxiety, post-traumatic stress disorder (PTSD), Schizophrenia (SZ), attention deficit hyperactivity disorder (ADHD), autism and/or an autism related disorder such as selected from the group consisting of Rett syndrome, Fragile X syndrome and Angelman syndrome³⁴⁻³⁷, whereas SorCS2 has been described as a regulator of BDNF-signaling, which is important for the development of depression. SorCS3 also has a strong genetic link to dementia. In one embodiment, the present invention relates to a cyclic peptide as disclosed herein for use in the treatment and/or prevention of mental and behavioural disorders.

In one embodiment, the mental and behavioural disorder is selected from the group consisting of depression, anxiety, post-traumatic stress disorder (PTSD), Schizophrenia (SZ), attention deficit hyperactivity disorder (ADHD), autism, Rett syndrome, Fragile X syndrome and Angelman syndrome.

In one embodiment, the present invention relates to a cyclic peptide of SEQ ID NO: 1, 2, 6 or 7 for use in the treatment and/or prevention of depression.

In one embodiment, the present invention relates to a cyclic peptide of SEQ ID NO: 1, 2, 6 or 7, or a pharmaceutically acceptable salt thereof, for use in the treatment and/or prevention of depression.

In one embodiment, the present invention relates to a cyclic peptide of SEQ ID NO: 6 for use in the treatment and/or prevention of a disease or disorder selected from the group consisting of anxiety, post-traumatic stress disorder (PTSD), Schizophrenia (SZ), attention deficit hyperactivity disorder (ADHD) and autism. In one embodiment, the present invention relates to a cyclic peptide of SEQ ID NO: 7 for use in the treatment and/or prevention of a disease or disorder selected from the group consisting of anxiety, post-traumatic stress disorder (PTSD), Schizophrenia (SZ), attention deficit hyperactivity disorder (ADHD) and autism.

In one embodiment, the present invention relates to a cyclic peptide of SEQ ID NO: 6, or a pharmaceutically acceptable salt thereof, for use in the treatment and/or prevention of a disease or disorder selected from the group consisting of anxiety, post-traumatic stress disorder (PTSD), Schizophrenia (SZ), attention deficit hyperactivity disorder (ADHD) and autism. In one embodiment, the present invention relates to a cyclic peptide of SEQ ID NO: 7, or a pharmaceutically acceptable salt thereof, for use in the treatment and/or prevention of a disease or disorder selected from the group consisting of anxiety, post-traumatic stress disorder (PTSD), Schizophrenia (SZ), attention deficit hyperactivity disorder (ADHD) and autism.

In one embodiment, the mental and behavioural disorder is depression. In one embodiment, the mental and behavioural disorder is autism or an autism related disorder, such as selected from the group consisting of Rett syndrome, Fragile X syndrome and Angelman syndrome. In one embodiment, the cyclic peptide of SEQ ID NO: 6 is for use in the treatment or prevention of is autism or an autism related disorder. In one embodiment, the cyclic peptide of SEQ ID NO: 7 is for use in the treatment or prevention of autism or an autism related disorder.

In one embodiment, the cyclic peptide of SEQ ID NO: 6, or a pharmaceutically acceptable salt thereof, is for use in the treatment or prevention of is autism or an autism related disorder. In one embodiment, the cyclic peptide of SEQ ID NO: 7, or a pharmaceutically acceptable salt thereof, is for use in the treatment or prevention of autism or an autism related disorder.

Neuropathic Pain

As demonstrated in Examples 28 and 29, a cyclic peptide of SEQ ID NO: 3 reduced neuropathic pain in a spared nerve injury mouse model. Hence, in one embodiment, the cyclic peptide of SEQ ID NO: 3 is for use in the treatment of neuropathic pain. In one embodiment, the cyclic peptide of SEQ ID NO: 3, or a pharmaceutically acceptable salt thereof, is for use in the treatment of neuropathic pain.

Neuropathic pain is a category of pain that includes several forms of chronic pain and which results from dysfunction of nervous rather than somatic tissue. Neuropathic pain, that is pain deriving from dysfunction of the central or peripheral nervous system, may also be a consequence of damage to peripheral nerves or to regions of the central nervous system, may result from disease, or may be idiopathic. Symptoms of neuropathic pain include sensations of burning, tingling, electricity, pins and needles, paresthesia, dysesthesia, stiffness, numbness in the extremities, feelings of bodily distortion, allodynia (pain evoked by stimulation that is normally innocuous), hyperalgesia (abnormal sensitivity to pain), hyperpathia (an exaggerated pain response persisting long after the pain stimuli cease), phantom pain, and spontaneous pain.

Stroke

The cyclic peptide of SEQ ID NO: 3 ameliorates neuropathic pain in mice demonstrated in Example 28 and 29. The cyclic peptide of SEQ ID NO: 1-3 is derived from SorCS2, which has been shown to have a functional link to stroke and epilepsy^{27,28}. In one embodiment, the present invention relates to a cyclic peptide as disclosed herein, such as a cyclic peptide of SEQ ID NO: 1, 2 or 3, for use in the treatment and/or prevention of stroke. In one embodiment, the present invention relates to a cyclic peptide as disclosed herein, such as a cyclic peptide of SEQ ID NO: 1, 2 or 3, or a pharmaceutically acceptable salt thereof, for use in the treatment and/or prevention of stroke.

Metabolic Disorders

The cyclic peptide of SEQ ID NO: 4 and 5 is derived from SorCS1, which has a strong genetic to metabolic diseases as diabetes mellitus type 1, diabetes mellitus type 2, obesity and/or nonalcoholic fatty liver disease (NAFLD)²⁹⁻³³. In one embodiment, the present invention relates to a cyclic peptide as disclosed herein, such as a cyclic peptide of SEQ ID NO: 4 or 5, for use in the treatment and/or prevention of a metabolic disorder. In one embodiment, the metabolic disorder is obesity. In one embodiment, the metabolic disorder is diabetes mellitus type 1. In one embodiment, the metabolic disorder is diabetes mellitus type 2. In one embodiment, the metabolic disorder is non-alcoholic fatty liver disease (NAFLD). In one embodiment, the metabolic disorder is a lysosomal storage disorder, such as Nieman-Pick disease.

In one embodiment, the present invention relates to a cyclic peptide as disclosed herein, such as a cyclic peptide of SEQ ID NO: 4 or 5, or a pharmaceutically acceptable salt thereof, for use in the treatment and/or prevention of a metabolic disorder. In one embodiment, the metabolic disorder is obesity. In one embodiment, the metabolic disorder is diabetes mellitus type 1. In one embodiment, the metabolic disorder is diabetes mellitus type 2. In one embodiment, the metabolic disorder is non-alcoholic fatty liver disease (NAFLD). In one embodiment, the metabolic disorder is a lysosomal storage disorder, such as Nieman-Pick disease.

Administration

According to the present invention, a cyclic peptide, or a composition comprising a cyclic peptide as defined herein, is administered to individuals in need of treatment in pharmaceutically effective doses or a therapeutically effective amount. A therapeutically effective amount is an amount which is sufficient to achieve a therapeutic effect. The dosage requirements will vary with the particular peptide composition employed, the route of administration and the particular subject being treated, which depend on the severity and the sort of the disorder as well as on the weight and general state of the subject. It will also be recognized by one skilled in the art that the optimal quantity and spacing of individual dosages of a peptide of the invention will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a peptide of the invention given per day for a defined number of days, can be ascertained using conventional course of treatment determination tests.

In one embodiment of the present invention, the cyclic peptide is administered in doses of from 1 μg/day to 100 mg/day. In one embodiment of the present invention, one single dose of cyclic peptide is administered and may comprise of from 1 μg/kg body weight to 100 mg/kg body weight, such as 1 μg/kg body weight to 10 mg/kg body weight. A preferred dose is about 0.1 mg/kg to about 10 mg/kg and an especially preferred dose is about 0.1 mg/kg to about 5 mg/kg. A dose according to the present invention may be administered one or several times per day. A dose may also be administered in intermittent intervals, or intervals, whereby a dose is not administered every day. Rather one or more doses may be administered every second day, every third day, every fourth day, every fifth day, every sixth day, every week, every second week, every third week, every fourth week, every fifth week, every sixth week, or intervals within those ranges (such as every 2 to 4 weeks, or 4 to 6 weeks).

It will be appreciated that the preferred route of administration will depend on the general condition and age of the subject to be treated, the nature of the condition to be treated, the location of the tissue to be treated in the body and the peptide of the invention chosen.

In one embodiment of the present invention, the route of administration allows for the cyclic peptide to cross the blood-brain barrier.

For systemic treatment according to the present invention the route of administration is capable of introducing the cyclic peptide into the blood stream to ultimately target the sites of desired action. Such routes of administration are any suitable routes, such as a parenteral route (including subcutaneous, intramuscular, intrathecal, intracerebral, intravenous and intradermal administration). Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration or subcutaneous administration.

In one embodiment administration is subcutaneously, such as by injection. In one embodiment administration is intramuscularly, such as by injection. In one embodiment administration is administered intradermally, such as by injection. In one embodiment administration is intravenously, such as by injection.

Pharmaceutical Composition

Whilst it is possible for the cyclic peptides of the present invention to be administered as the raw peptide, it is preferred to present them in the form of a pharmaceutical formulation. Accordingly, the present invention further provides a pharmaceutical formulation, which comprises a cyclic peptide of the present invention or a pharmaceutically acceptable salt thereof, and a pharmaceutically acceptable carrier therefore. Thus, in one aspect, the present invention concerns a composition, such as a pharmaceutical composition, comprising the peptide as defined herein. The pharmaceutical formulations may be prepared by conventional techniques, e.g. as described in Remington: The Science and Practice of Pharmacy 2005, Lippincott, Williams & Wilkins.

Pharmaceutically acceptable carriers include water.

A pharmaceutically acceptable composition for parenteral administration should have a physiologically acceptable pH and should have a physiologically acceptable osmolality.

The pH of an aqueous composition may be adjusted in view of the components of the composition and necessary suitability for administration. The pH is generally at least 4, especially at least 5, in particular at least 5.5 such as at least 6. The pH is generally 9 or less, especially 8.5 or less, in particular 8 or less, such as 7.5 or less. The pH of may be 4 to 9, especially 5 to 8.5, in particular 5.5 to 8, such as 6.5 to 7.4 (e.g. 6.5 to 7.1).

For parenteral administration a physiologically acceptable osmolality is desirable to avoid excessive cell distortion or lysis. A physiologically acceptable osmolality will generally mean that solutions will have an osmolality which is approximately isotonic or mildly hypertonic. Suitably compositions for administration will have an osmolality of 250 to 750 mOsm/kg, especially 250 to 550 mOsm/kg, in particular 270 to 500 mOsm/kg, such as 270 to 400 mOsm/kg.

Other components, such as buffers or stabilizing agents, may also be present.

As used herein, “pharmaceutically acceptable salts” refer to derivatives wherein a peptide of the invention is modified by making pharmaceutically acceptable acid or base salts thereof. The phrase “pharmaceutically acceptable salt” is employed herein to refer to those salts which are, within the scope of sound medical judgment, suitable for use in a pharmaceutical context, without excessive toxicity, irritation, allergic response, or other problem or complication, commensurate with a reasonable benefit/risk ratio. Non-pharmaceutically acceptable salts may nevertheless be of utility during the manufacture of a peptide of the invention or a pharmaceutically acceptable salt thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. Lists of suitable salts are found in, for example, Remington's Pharmaceutical Sciences, 17th ed., Mack Publishing Company, Easton, P A, 1985, p. 1418, the disclosure of which is hereby incorporated by reference.

The invention is further illustrated by reference to the following clauses:

Clause A1. A cyclic peptide comprising or consisting of an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

Clause A2. The cyclic peptide according to clause A1, wherein the cyclic peptide consists of the amino acid sequence of MTEPVEHEEDV (SEQ ID NO: 1).

Clause A3. The cyclic peptide according to clause A1, wherein the cyclic peptide consists of the amino acid sequence of MTDPVDHDEDV (SEQ ID NO: 2).

Clause A4. The cyclic peptide according to clause A1, wherein the cyclic peptide consists of the amino acid sequence of MTAPVAHAEDV (SEQ ID NO: 3).

Clause A5. The cyclic peptide according to clause A1, wherein the cyclic peptide consists of the amino acid sequence of MIEPVEHEESR (SEQ ID NO: 4).

Clause A6. The cyclic peptide according to clause A1, wherein the cyclic peptide consists of the amino acid sequence of MIDPVDHDESR (SEQ ID NO: 5).

Clause A7. The cyclic peptide according to clause A1, wherein the cyclic peptide consists of the amino acid sequence of MIGSVEQEENA (SEQ ID NO: 6).

Clause A8. The cyclic peptide according to clause A1, wherein the cyclic peptide consists of the amino acid sequence of MIGSVDQDENA (SEQ ID NO: 7).

Clause A9. The cyclic peptide according to any one of the preceding clauses, wherein the cyclic peptide is a backbone cyclized peptide.

Clause A10. The cyclic peptide according to clause A1, wherein the cyclic peptide consists of no more than 20 amino acid residues, such as no more than 15 amino acid residues, such as no more than 14 amino acid residues, such as no more than 13 amino acid residues, such as no more than 12 amino acid residues.

Clause A11. The cyclic peptide according to any one of the preceding clauses, wherein the peptide is further conjugated to a detectable moiety.

Clause A12. A composition comprising the peptide according to any one of the preceding clauses.

Clause A13. The composition according to clause A12, wherein the composition is a pharmaceutical composition.

Clause A14. The cyclic peptide according to any one of clauses A1 to A11 or the composition according to any one of clauses A12 to A13 for use as a medicament.

Clause A15. The cyclic peptide according to any one of clauses A1 to A11 or the composition according to any one of clauses A12 to A13 for use in the treatment and/or prevention of a disease or disorder selected from the group consisting of diseases of the nervous system; neuropathic pain; mental and behavioural disorders; stroke and metabolic disorders.

Clause A16. The cyclic peptide according to any one of clauses A1 to A11 or the composition according to any one of clauses A12 to A13 for use according to clause A15, wherein the diseases of the nervous system is selected from the group consisting of Huntington's disease, amyotrophic lateral sclerosis (ALS), Parkinson's disease, Alzheimer's disease, Frontotemporal dementia (FTD) and epilepsy.

Clause A17. The cyclic peptide according to any one of clauses A1 to A11 or the composition according to any one of clauses A12 to A13 for use according to clause A15, wherein the diseases of the nervous system is a neurodegenerative disease.

Clause A18. The cyclic peptide for use according to clause A17, wherein the neurodegenerative disease is selected from the group consisting of Frontotemporal dementia (FTD), Huntington's disease, Alzheimer's disease, Parkinson's disease and amyotrophic lateral sclerosis.

Clause A19. The cyclic peptide according to any one of clauses A1 to A11 or the composition according to any one of clauses A12 to A13 for use according to clause A15, wherein the mental and behavioural disorder is selected from the group consisting of depression, anxiety, post-traumatic stress disorder (PTSD), Schizophrenia (SZ), attention deficit hyperactivity disorder (ADHD), autism, Rett syndrome, Fragile X syndrome and Angelman syndrome.

Clause A20. The cyclic peptide according to any one of clauses A1 to A11 or the composition according to any one of clauses A12 to A13 for use according to clause A15, wherein the metabolic disorder is selected from the group consisting of obesity; diabetes mellitus type 1; diabetes mellitus type 2 and non-alcoholic fatty liver disease (NAFLD).

Clause A21. The cyclic peptide according to clause A2 for use in the treatment or prevention of Huntington's disease, amyotrophic lateral sclerosis (ALS), Parkinson's disease, Alzheimer's disease, Frontotemporal dementia (FTD), depression and/or stroke.

Clause A22. The cyclic peptide according to clause A3 for use in the treatment or prevention of Huntington's disease, amyotrophic lateral sclerosis (ALS), Parkinson's disease, Alzheimer's disease, Frontotemporal dementia (FTD), depression and/or stroke.

Clause A23. The cyclic peptide according to clause A4 for use in the treatment or prevention of neuropathic pain, stroke and/or epilepsy.

Clause A24. The cyclic peptide according to clause A5 for use in the treatment or prevention of Huntington's disease, Parkinson's disease, Alzheimer's disease, Frontotemporal dementia (FTD), diabetes mellitus type 1, diabetes mellitus type 2, obesity and/or nonalcoholic fatty liver disease (NAFLD).

Clause A25. The cyclic peptide according to clause A6 for use in the treatment or prevention of Huntington's disease, Parkinson's disease, Alzheimer's disease, Frontotemporal dementia (FTD), diabetes mellitus type 1, diabetes mellitus type 2, obesity and/or non-alcoholic fatty liver disease (NAFLD).

Clause A26. The cyclic peptide according to clause A7 for use in the treatment or prevention of Huntington's disease, Parkinson's disease, Alzheimer's disease, Frontotemporal dementia (FTD), depression, anxiety, post-traumatic stress disorder (PTSD), Schizophrenia (SZ), attention deficit hyperactivity disorder (ADHD), autism and/or an autism related disorder, such as selected from the group consisting of Rett syndrome, Fragile X syndrome and Angelman syndrome.

Clause A27. The cyclic peptide according to clause A8 for use in the treatment or prevention of Huntington's disease, Parkinson's disease, Alzheimer's disease, Frontotemporal dementia (FTD), depression, anxiety, post-traumatic stress disorder (PTSD), Schizophrenia (SZ), attention deficit hyperactivity disorder (ADHD), autism and/or an autism related disorder, such as selected from the group consisting of Rett syndrome, Fragile X syndrome and Angelman syndrome.

Clause A28. A method of treatment or prevention of a disease or disorder selected from the group consisting of diseases of the nervous system; neuropathic pain; mental and behavioural disorders; stroke and metabolic disorders, said method comprising administering the cyclic peptide according to any one of clauses A1 to A11 or the composition according to any one of clauses A12 to A13 to a subject in need thereof.

Clause A29. Use of the cyclic peptide according to any one of clauses A1 to A11 or the composition according to any one of clauses A12 to A13 for the manufacture of a medicament for the treatment and/or prevention of a disease or disorder selected from the group consisting of diseases of the nervous system; neuropathic pain; mental and behavioural disorders; stroke and metabolic disorders.

Clause A30. A method of increasing the number of synapses, said method comprising the administration of the peptide according to any one of clauses A1 to A11 or the composition according to any one of clauses A12 to A13 to a subject in need thereof.

Clause A31. A method of manufacturing the cyclic peptide according to any one of clauses A1 to A11, said method comprising the steps of
a) Preparing a linear peptide of an amino acid sequence selected from the group consisting of SEQ ID NOs: 1 to 7; and
b) Cyclizing the peptide of a) to generate a backbone cyclized peptide.

Clause A32. The method according to clause A31, wherein the linear peptide of a) is prepared by recombinantly expressing the peptide, for example in an E. coli system.

Clause A33. The method according to clause A31, wherein the linear peptide of a) is prepared synthetically.

Clause 1. A cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof.

Clause B2. The pharmaceutically acceptable salt of a cyclic peptide according to clause E1.

Clause B3. The cyclic peptide according to clause E1.

Clause B4. The cyclic peptide or pharmaceutically acceptable salt according to any one of clauses 1 to B3, wherein the peptide is side chain-to-side chain cyclised.

Clause B5. The cyclic peptide or pharmaceutically acceptable salt according to any one of clauses 1 to B3, wherein the peptide is tail-to-side chain cyclised.

Clause B6. The cyclic peptide or pharmaceutically acceptable salt according to any one of clauses 1 to B3, wherein the peptide is side chain-to-head cyclised.

Clause B7. The cyclic peptide or pharmaceutically acceptable salt according to any one of clause B4 to B6, wherein the backbone of the peptide is joined exclusively by peptide bonds.

Clause B8. The cyclic peptide or pharmaceutically acceptable salt according to any one of clause 1 to B3, wherein the peptide is backbone cyclised.

Clause B9. The cyclic peptide or pharmaceutically acceptable salt according to clause B8, wherein the backbone of the peptide is joined exclusively by peptide bonds.

Clause B10. The cyclic peptide or pharmaceutically acceptable salt according to clause B9, wherein all residues of the peptide are joined exclusively by peptide bonds.

Clause B11. The cyclic peptide or pharmaceutically acceptable salt according to any one of clauses 1 to B10, wherein the cyclic peptide comprises no more than 50 amino acid residues, such as no more than 40 amino acid residues, such as no more than 30 amino acid residues, such as no more than 20 amino acid residues.

Clause B12. The cyclic peptide or pharmaceutically acceptable salt according to clause B11, wherein the cyclic peptide comprises no more than 14 amino acid residues, such as no more than 13 amino acid residues, such as no more than 12 amino acid residues.

Clause B13. The cyclic peptide or pharmaceutically acceptable salt according to any one of clauses 1 to B12, wherein the cyclic peptide is modified.

Clause B14. The cyclic peptide or pharmaceutically acceptable salt according to clause B13, wherein the cyclic peptide is conjugated to a detectable moiety.

Clause B15. The cyclic peptide or pharmaceutically acceptable salt according to any one of clauses 1 to B12, wherein the cyclic peptide is not modified.

Clause B16. The cyclic peptide or pharmaceutically acceptable salt according to any one of clauses 1 to B15, wherein the cyclic peptide comprises the amino acid sequence of MTEPVEHEEDV (SEQ ID NO: 1).

Clause B17. The cyclic peptide or pharmaceutically acceptable salt according to clause B16, wherein the cyclic peptide consists of the amino acid sequence of MTEPVEHEEDV (SEQ ID NO: 1).

Clause B18. The cyclic peptide or pharmaceutically acceptable salt according to any one of clauses 1 to B15, wherein the cyclic peptide comprises the amino acid sequence of MTDPVDHDEDV (SEQ ID NO: 2).

Clause B19. The cyclic peptide or pharmaceutically acceptable salt according to clause B18, wherein the cyclic peptide consists of the amino acid sequence of MTDPVDHDEDV (SEQ ID NO: 2).

Clause B20. The cyclic peptide or pharmaceutically acceptable salt according to any one of clauses 1 to B15, wherein the cyclic peptide comprises the amino acid sequence of MTAPVAHAEDV (SEQ ID NO: 3).

Clause B21. The cyclic peptide or pharmaceutically acceptable salt according to clause B20, wherein the cyclic peptide consists of the amino acid sequence of MTAPVAHAEDV (SEQ ID NO: 3).

Clause B22. The cyclic peptide or pharmaceutically acceptable salt according to any one of clauses 1 to B15, wherein the cyclic peptide comprises the amino acid sequence of MIEPVEHEESR (SEQ ID NO: 4).

Clause B23. The cyclic peptide or pharmaceutically acceptable salt according to clause B22, wherein the cyclic peptide consists of the amino acid sequence of MIEPVEHEESR (SEQ ID NO: 4).

Clause B24. The cyclic peptide or pharmaceutically acceptable salt according to any one of clauses 1 to B15, wherein the cyclic peptide comprises the amino acid sequence of MIDPVDHDESR (SEQ ID NO: 5).

Clause B25. The cyclic peptide or pharmaceutically acceptable salt according to clause B24, wherein the cyclic peptide consists of the amino acid sequence of MIDPVDHDESR (SEQ ID NO: 5).

Clause B26. The cyclic peptide or pharmaceutically acceptable salt according to any one of clauses 1 to B15, wherein the cyclic peptide comprises the amino acid sequence of MIGSVEQEENA (SEQ ID NO: 6).

Clause B27. The cyclic peptide or pharmaceutically acceptable salt according to clause B26, wherein the cyclic peptide consists of the amino acid sequence of MIGSVEQEENA (SEQ ID NO: 6).

Clause B28. The cyclic peptide or pharmaceutically acceptable salt according to any one of clauses 1 to B15, wherein the cyclic peptide comprises the amino acid sequence of MIGSVDQDENA (SEQ ID NO: 7).

Clause B29. The cyclic peptide or pharmaceutically acceptable salt according to clause B28, wherein the cyclic peptide consists of the amino acid sequence of MIGSVDQDENA (SEQ ID NO: 7).

Clause B30. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MTEPVEHEEDV (SEQ ID NO: 1).

Clause B31. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MTDPVDHDEDV (SEQ ID NO: 2).

Clause B32. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MTAPVAHAEDV (SEQ ID NO: 3).

Clause B33. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MIEPVEHEESR (SEQ ID NO: 4).

Clause B34. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MIDPVDHDESR (SEQ ID NO: 5).

Clause B35. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MIGSVEQEENA (SEQ ID NO: 6).

Clause B36. The cyclic peptide or pharmaceutically acceptable salt according to clause E1, wherein the peptide is backbone cyclized, all residues of the peptide are joined exclusively by peptide bonds, the peptide is unmodified and consists of the amino acid sequence of MIGSVDQDENA (SEQ ID NO: 7).

Clause B37. An aqueous composition comprising a cyclic peptide or pharmaceutically acceptable salt according to any one of clauses 1 to B36.

Clause B38. A pharmaceutical composition comprising a cyclic peptide or pharmaceutically acceptable salt according to any one of clauses 1 to B36.

Clause B39. The cyclic peptide or pharmaceutically acceptable salt according to any one of clauses 1 to B36, or the pharmaceutical composition according to clause B38, for use as a medicament.

Clause B40. Use of cyclic peptide or pharmaceutically acceptable salt according to any one of clauses 1 to B36, or a pharmaceutical composition according to clause B38, for the manufacture of a medicament.

Clause B41. The cyclic peptide, pharmaceutically acceptable salt or pharmaceutical composition according to clause B39, for use in the therapy of a disease of the nervous system; neuropathic pain; a mental or behavioural disorder; stroke; or a metabolic disorder.

Clause B42. The use according to clause B40, for the manufacture of a medicament for the therapy of a disease of the nervous system; neuropathic pain; a mental or behavioural disorder; stroke or a metabolic disorder.

Clause B43. A method of therapy of a disease of the nervous system; neuropathic pain; a mental or behavioural disorder; stroke; or a metabolic disorder, said method comprising administering to a subject the cyclic peptide or pharmaceutically acceptable salt according to any one of clauses 1 to B36, or a pharmaceutical composition according to clause B38.

Clause B44. The cyclic peptide, pharmaceutically acceptable salt or pharmaceutical composition according to clause B39, for use in the prophylaxis of a disease of the nervous system; neuropathic pain; a mental or behavioural disorder; stroke; or a metabolic disorder.

Clause B45. The use according to clause B40, for the manufacture of a medicament for the prophylaxis of a disease of the nervous system; neuropathic pain; a mental or behavioural disorder; stroke or a metabolic disorder.

Clause B46. A method of prophylaxis of a disease of the nervous system; neuropathic pain; a mental or behavioural disorder; stroke; or a metabolic disorder, said method comprising administering to a subject the cyclic peptide or pharmaceutically acceptable salt according to any one of clauses 1 to B36, or a pharmaceutical composition according to clause B38.

Clause B47. The cyclic peptide, pharmaceutically acceptable salt according, pharmaceutical composition, method or use according to any one of clauses clause B39 to B43, for the therapy of Huntington's disease.

Clause B48. The cyclic peptide, pharmaceutically acceptable salt according, pharmaceutical composition, method or use according to any one of clauses clause B39 or B44 to B46, for the prophylaxis of Huntington's disease.

Clause B49. The cyclic peptide, pharmaceutically acceptable salt according, pharmaceutical composition, method or use according to any one of clauses clause B39 to B43, for the therapy of frontotemporal dementia.

Clause B50. The cyclic peptide, pharmaceutically acceptable salt according, pharmaceutical composition, method or use according to any one of clauses clause B39 or B44 to B46, for the prophylaxis of frontotemporal dementia.

Clause B51. The cyclic peptide, pharmaceutically acceptable salt according, pharmaceutical composition, method or use according to any one of clauses clause B39 to B43, for the therapy of Parkinson's disease.

Clause B52. The cyclic peptide, pharmaceutically acceptable salt according, pharmaceutical composition, method or use according to any one of clauses clause B39 or B44 to B46, for the prophylaxis of Parkinson's disease.

Clause B53. The cyclic peptide, pharmaceutically acceptable salt according, pharmaceutical composition, method or use according to any one of clauses clause B39 to B43, for the therapy of lysosomal storage disorders, such as Nieman-Pick disease.

Clause B54. The cyclic peptide, pharmaceutically acceptable salt according, pharmaceutical composition, method or use according to any one of clauses clause B39 or B44 to B46, for the prophylaxis of lysosomal storage disorders, such as Nieman-Pick disease.

Clause B55. The cyclic peptide, pharmaceutically acceptable salt according, pharmaceutical composition, method or use according to any one of clauses clause B39 to B43, for the therapy of WAGR syndrome.

Clause B56. The cyclic peptide, pharmaceutically acceptable salt according, pharmaceutical composition, method or use according to any one of clauses clause B39 or B44 to B46, for the prophylaxis of WAGR syndrome.

Clause B57. The cyclic peptide, pharmaceutically acceptable salt according, pharmaceutical composition, method or use according to any one of clauses clause B39 to B43, for the therapy of dementia.

Clause B58. The cyclic peptide, pharmaceutically acceptable salt according, pharmaceutical composition, method or use according to any one of clauses clause B39 or B44 to B46, for the prophylaxis of dementia.

Clause B59. The cyclic peptide, pharmaceutically acceptable salt according, pharmaceutical composition, method or use according to any one of clauses clause B38 to B58, for administration at 1 μg/day to 100 mg/day, such as 0.1 mg to 10 mg/day.

Clause B60. The cyclic peptide, pharmaceutically acceptable salt according, pharmaceutical composition, method or use according to any one of clauses clause B38 to B59, for administration subcutaneously, such as by injection.

Clause B61. The cyclic peptide, pharmaceutically acceptable salt according, pharmaceutical composition, method or use according to any one of clauses clause B38 to B59, for administration intramuscularly, such as by injection.

Clause B62. The cyclic peptide, pharmaceutically acceptable salt according, pharmaceutical composition, method or use according to any one of clauses clause B38 to B59, for administration intravenously, such as by injection.

Clause B63. The cyclic peptide, pharmaceutically acceptable salt according, pharmaceutical composition, method or use according to any one of clauses clause B38 to B59, for administration to a human subject.

Clause B64. A salt of a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7.

Clause B65. A method of manufacturing a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7, or a salt thereof, the method comprising the steps of:
(i) preparing a linear peptide, or a salt thereof, having an appropriate amino acid sequence, and
(ii) subsequently generating a cyclized peptide, or a salt thereof, from the linear peptide.

Clause B66. A method of manufacturing a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7, or a salt thereof, the method comprising the steps of:
(i) preparing a protected linear peptide, or a salt thereof, having an appropriate amino acid sequence;
(ii) subsequently generating a protected cyclized peptide, or a salt thereof, from the protected linear peptide; and
(iii) removing protecting groups to provide a cyclized peptide, or a salt thereof.

Clause B67. A linear amino acid sequence selected from the group consisting of SEQ ID NO: 10 to 16 and 20 to 89, a salt thereof, or a protected version thereof.

Clause B68. A nucleic acid construct encoding for and being capable of expressing a peptide comprising an amino acid sequence selected from the group consisting of SEQ ID NO: 10 to 16 and 20 to 89.

Clause B69. The nucleic acid construct according to clause B68, encoding for and being capable of expressing a peptide consisting of an amino acid sequence selected from the group consisting of SEQ ID NO: 10 to 16 and 20 to 89.

Clause B70. A vector comprising the nucleic acid construct according to either clause B68 or B69.

Clause B71. An isolated host cell comprising the nucleic acid construct according to either clause B68 or B69 or a vector according to clause B70.

Clause B72. The host cell according to clause B71 which is a bacterial cell.

### EXAMPLES

The following peptides are studied in the Examples:

| Peptide | Sequence | SEQ ID NO | |
|---|---|---|---|
| | ...MTSPVSHSEDV... | 17 | Native SorCS2 sequence |
| P1 | [MTEPVEHEEDV] | 1 | Cyclic (backbone) |
| P2 | [MTDPVDHDEDV] | 2 | Cyclic (backbone) |
| P3 | [MTAPVAHAEDV] | 3 | Cyclic (backbone) |
| | ...MISPVSHSESR... | 18 | Native SorCS1 sequence |
| P4 | [MIEPVEHEESR] | 4 | Cyclic (backbone) |
| P5 | [MIDPVDHDESR] | 5 | Cyclic (backbone) |
| | ...MIGSVSQSENA... | 19 | Native SorCS3 sequence |
| P6 | [MIGSVEQEENA] | 6 | Cyclic (backbone) |
| P7 | [MIGSVDQDENA] | 7 | Cyclic (backbone) |
| | ...MTSPVSHSEDV... | 17 | Native SorCS2 sequence |
| LP1 | Ac-MTEPVEHEEDV—NH₂ | 8 | Linear, amidated and |
| | | | acetylated N- and C- |
| | | | terminals |
| P9 | YARAAARNARAEKEQEMTDP | 9 | Linear, longer and has cell- |
| | VDHDEDVQGAVQ | | penetrating moiety (TAT- |
| | | | sequence) |
| Scr | YARAAARNARAMDRTVQKVF | 90 | Scrambled peptide |
| | EHQENYRIYVKRGDPATEAQ | | connected to cell- |
| | DKFALAKGHEGVQPDM | | penetrating moiety (TAT- |
| | | | sequence) |

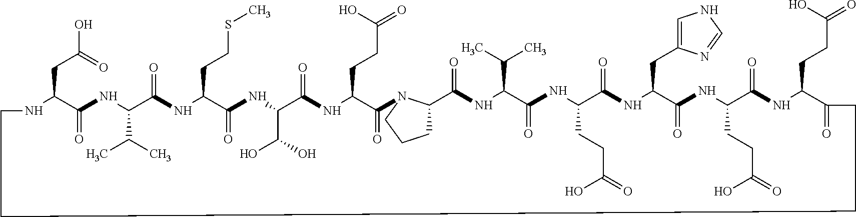

P1 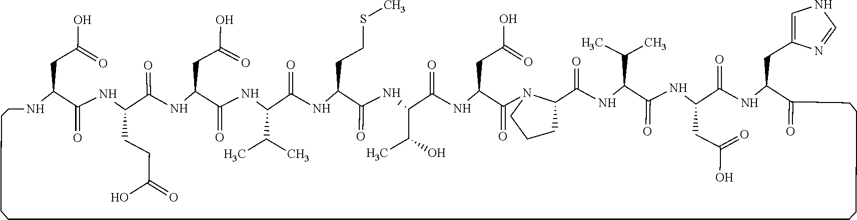 [SMILES: CSCC[C@@H]1NC(=O)[C@H](C(C)C)NC(=O)[C@H](CC(=O)O)NC(=O)[C@H](CCC(=O)O)NC(=O)[C@H](CC(=O)O)NCCCCCCCCC(=O)[C@H](Cc2c[nH]cn2)NC(=O)[C@H](CC(=O)O)NC(=O)[C@H](C(C)C)NC(=O)[C@@H]2CCCN2C(=O)[C@H](CC(=O)O)NC(=O)[C@H]([C@@H](C)O)NC1=O]
P2 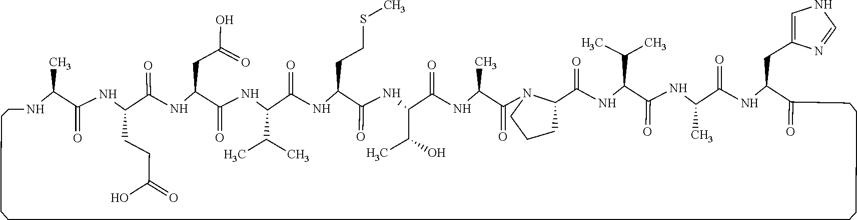 [SMILES: CSCC[C@@H]1NC(=O)[C@H](C(C)C)NC(=O)[C@H](CC(=O)O)NC(=O)[C@H](CCC(=O)O)NC(=O)[C@H](C)NCCCCCCCCC(=O)[C@H](Cc2c[nH]cn2)NC(=O)[C@H](C)NC(=O)[C@H](C(C)C)NC(=O)[C@@H]2CCCN2C(=O)[C@H](C)NC(=O)[C@H]([C@@H](C)O)NC1=O]
P3 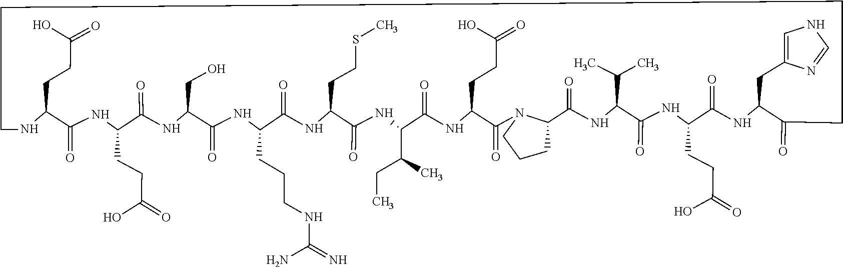 [SMILES: CC[C@H](C)[C@@H]1NC(=O)[C@H](CCSC)NC(=O)[C@H](CCCNC(=N)N)NC(=O)[C@H](CO)NC(=O)[C@H](CCC(=O)O)NC(=O)[C@H](CCC(=O)O)NCCCCC(=O)[C@H](Cc2c[nH]cn2)NC(=O)[C@H](CCC(=O)O)NC(=O)[C@H](C(C)C)NC(=O)[C@@H]2CCCN2C(=O)[C@H](CCC(=O)O)NC1=O]
P4 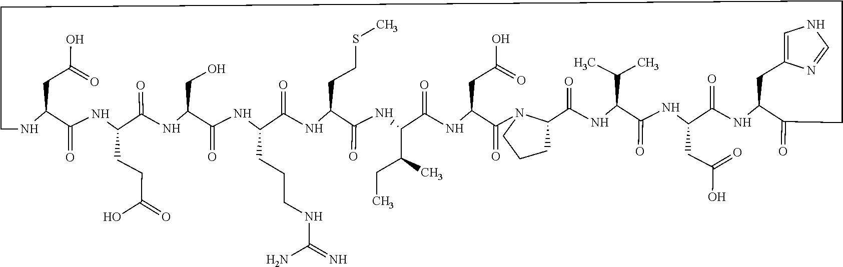 [SMILES: CC[C@H](C)[C@@H]1NC(=O)[C@H](CCSC)NC(=O)[C@H](CCCNC(=N)N)NC(=O)[C@H](CO)NC(=O)[C@H](CCC(=O)O)NC(=O)[C@H](CC(=O)O)NCCCCC(=O)[C@H](Cc2c[nH]cn2)NC(=O)[C@H](CC(=O)O)NC(=O)[C@H](C(C)C)NC(=O)[C@@H]2CCCN2C(=O)[C@H](CC(=O)O)NC1=O]
P5 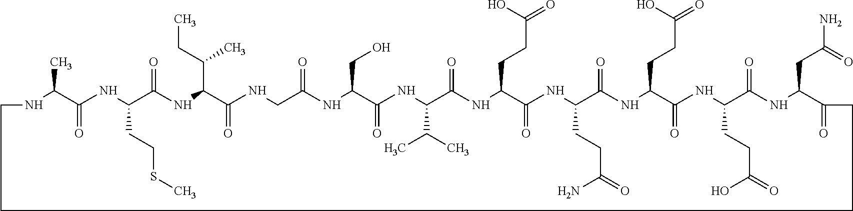 [SMILES: CC[C@H](C)[C@@H]1NC(=O)[C@H](CCSC)NC(=O)[C@H](C)NCCCCC(=O)[C@H](CC(N)=O)NC(=O)[C@H](CCC(=O)O)NC(=O)[C@H](CCC(=O)O)NC(=O)[C@H](CCC(N)=O)NC(=O)[C@H](CCC(=O)O)NC(=O)[C@H](C(C)C)NC(=O)[C@H](CO)NC(=O)CNC1=O]
P6 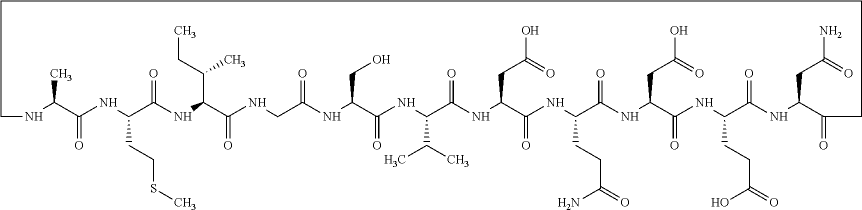 [SMILES: CC[C@H](C)[C@@H]1NC(=O)[C@H](CCSC)NC(=O)[C@H](C)NCCCCC(=O)[C@H](CC(N)=O)NC(=O)[C@H](CCC(=O)O)NC(=O)[C@H](CC(=O)O)NC(=O)[C@H](CCC(N)=O)NC(=O)[C@H](CC(=O)O)NC(=O)[C@H](C(C)C)NC(=O)[C@H](CO)NC(=O)CNC1=O]
P7 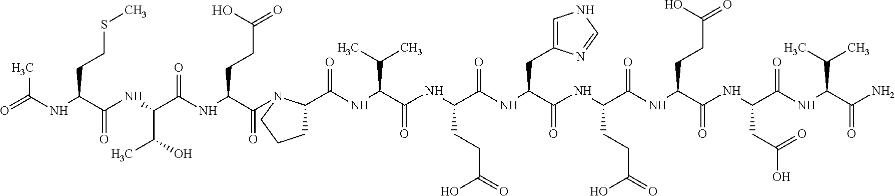 [SMILES: CSCC[C@H](NC(C)=O)C(=O)N[C@H](C(=O)N[C@@H](CCC(=O)O)C(=O)N1CCC[C@H]1C(=O)N[C@H](C(=O)N[C@@H](CCC(=O)O)C(=O)N[C@@H](Cc1c[nH]cn1)C(=O)N[C@@H](CCC(=O)O)C(=O)N[C@@H](CCC(=O)O)C(=O)N[C@@H](CC(=O)O)C(=O)N[C@H](C(N)=O)C(C)C)C(C)C)[C@@H](C)O]
LP1
P9 see FIG. 35

### Example 1: Peptide Synthesis

Linear peptides were synthesized using standard Fmoc (fluorenylmethyloxycarbonyl) chemistry.

Resin preparation: Fmoc-Pro-OH (0.2 mmol, 1 eq) and N,N-diisopropylethylamine (DIPEA) (0.14 mL, 4 eq) was added to the 2-CTC Resin (0.2 mmol, 1.00 eq, Sub 1.05 mmol/g) in dichloromethane (DCM) (10 mL). The mixture was agitated with N₂ for 2 h at 20° C., then methanol (MeOH) (0.5 mL) added and agitated with N₂ bubbling for another 30 min. The resin was washed three times with dimethylformamide (DMF) (15 mL). Deprotection: Fmoc removal was performed using 20% piperidine in DMF (15 mL) added and to the resin and agitated with N₂ for another 30 min. The resin was washed with DMF four times (15 mL) and filtered. Coupling: The consecutive amino acid couplings were performed using a solution of 2-(1H-benzotriazole-1-yl)-1,1,3,3-tetramethyluronium hexafluorophosphate (HBTU) (2.85 eq), DIPEA (6 eq) and Fmoc-protected amino acids (3 eq) in DMF (5 mL) added to the resin and agitated with N₂ for 30 min at 20° C. The resin was then washed four times with DMF (15 mL). Fmoc deprotection and coupling steps were repeated each of the following amino acids until the desired peptide sequence was achieved. The resin was then washed four times with dimethylformamide (DMF) (15 mL). Fmoc removal and coupling steps were repeated until the desired peptide sequence was achieved. The resultant side chain protected and resin bound linear peptide was used directly in the next step.

The Fmoc-protected amino acid building blocks used were: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Tyr(tBu)-OH, and Fmoc-L-Val-OH. If nothing else is specified, the natural L-form of the amino acids were used.

### Example 2: Peptide Cleavage, Cyclisation and Purification

After final amino acid coupling and Fmoc removal, the resin from Example 1 was washed with DMF 5 times, with MeOH 3 times, and dried under vacuum. The peptide resin was then treated with the cleavage cocktail (1% trifluoroacetic acid (TFA)/99% DCM) (15 mL) for 15 min and the peptide containing TFA-DCM mixture was collected. The cleavage was repeated three times.

The peptide (in 1% TFA/99% DCM) was diluted in DCM (200 mL) together with 2-(1H-benzotriazole-1-yl)-1,1,3,3-tetramethylaminium tetrafluoroborate (TBTU) (2 eq) and 1-hydroxybenzotriazole hydrate (HOBT) (2 eq) and DIPEA (6 eq) to couple the head to tail of the peptide. The mixture was stirred at 20° C. for 1 h.

LC-MS Method for Monitoring Cyclisation (Sidechain Protected Peptide):
System: Agilent Infinity II 1260 HPLC series
Column: Xbridge C18, 130 Å, 3.5 μm, 2.1×30 mm
Detector: Agilent LC-MS (G6125C), single quadrupole TIC scan
Scanning range: m/z min 100, m/z max. 2000, positive mode
Gradient: Gradient run-time 2 minutes; 0.00-2.00 min 70-100% B. Column cleaning and equilibration; 2.00-2.01100% B, 2.01-2.50 min 100% B, 2.50-2.51 min 100-10% B, 2.51-3.00 min 10% B.
Flow rate: 1.2 mL/min
Diode array: 220/254 nm
Column temperature: Room temperature
Solvent A: 0.1% TFA in water
Solvent B: 0.075% TFA in acetonitrile

After stirring, the mixture was washed with 1M hydrochloric acid (HCl) (30 mL) twice and dried under pressure to give the crude powder. 5 mL of cleavage buffer (92.5% TFA/2.5% 3-mercaptopropionic acid/2.5% triisopropyl silane/2.5% H₂O) was added to the flask containing the side chain protected cyclic peptide and the mixture was stirred for 2 h at 20° C. The peptide was precipitated with ice cold tert-butyl methyl ether (40 mL) and centrifuged (2 min at 3000 rpm) and washed two times with ice cold tert-butyl methyl ether (40 mL). The crude peptide was dried under vacuum for 2 hours and purified by prep-HPLC and the target peptide fraction freeze dried to give a white solid.

Prep-HPLC Method:
System: Gilson GX-281
Column: Gemini, C18, 110 Å, 5 μm or Luna, C18, 100 Å, 10 μm.
Gradient: Gradient run-time 50 minutes; 0 to 50 min 7 to 37% B.
Flow rate: 20 mL/min
Column temperature: 30° C.
Diode array: 220/254 nm
Solvent A: 0.075% TFA in water
Solvent B: Acetonitrile

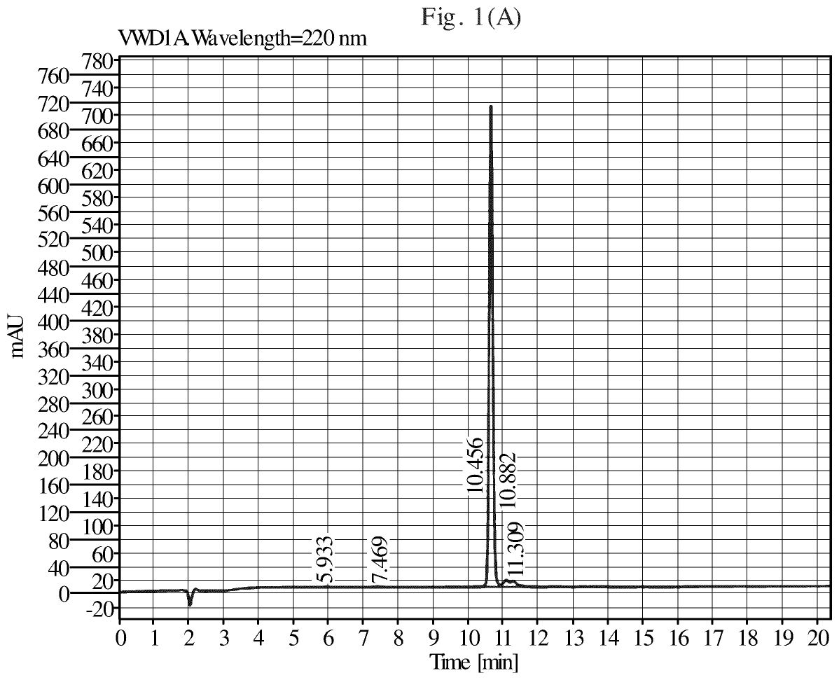

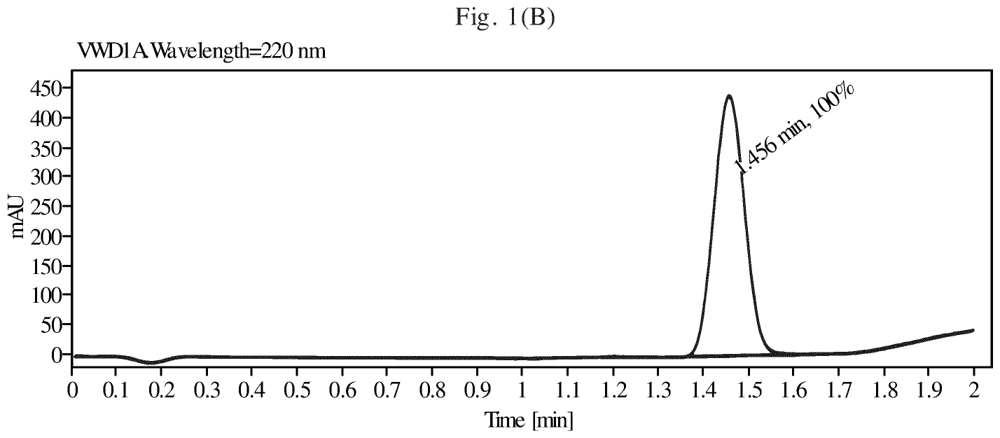

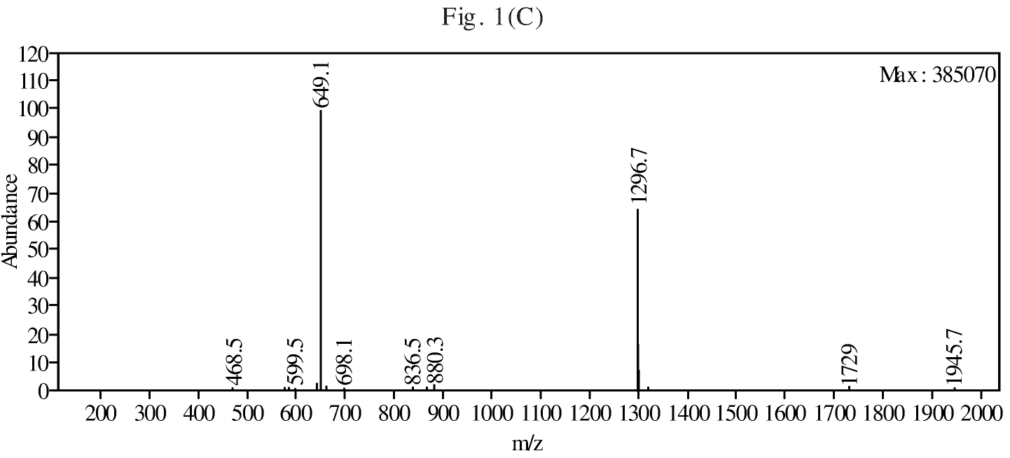

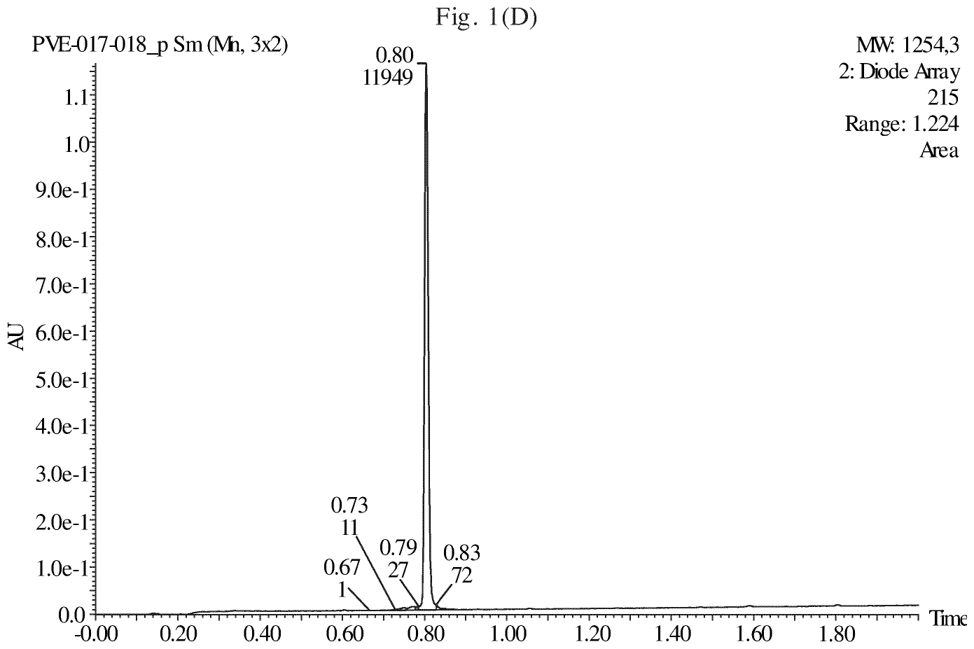

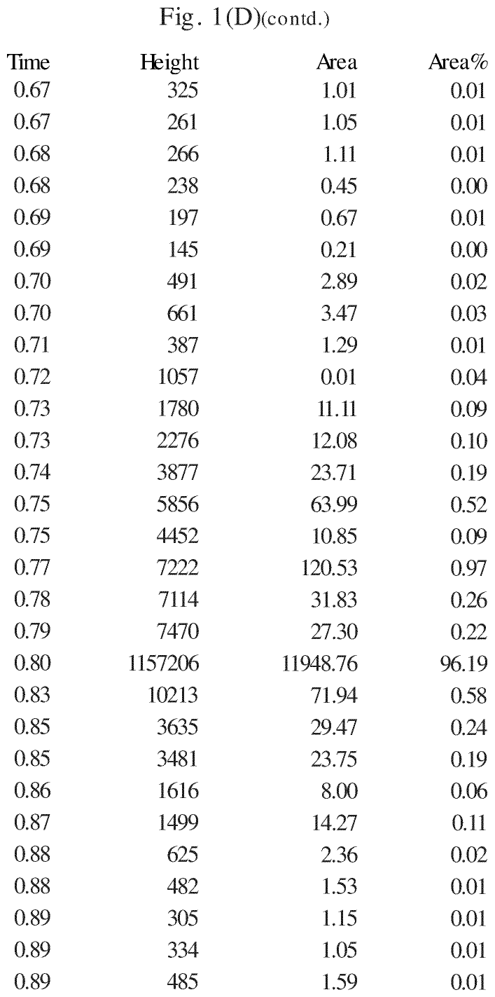

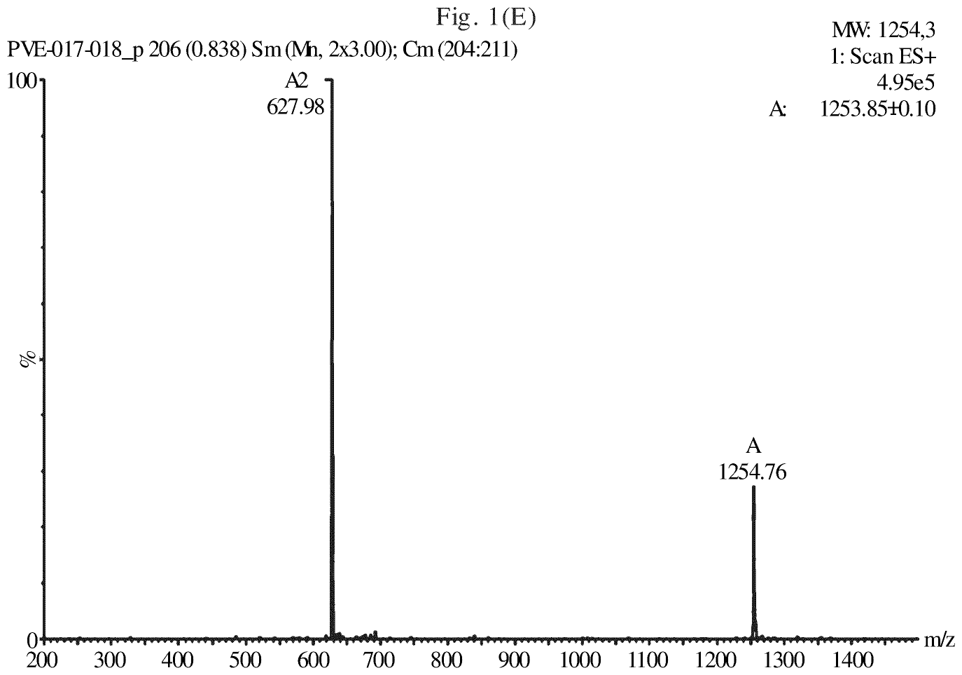

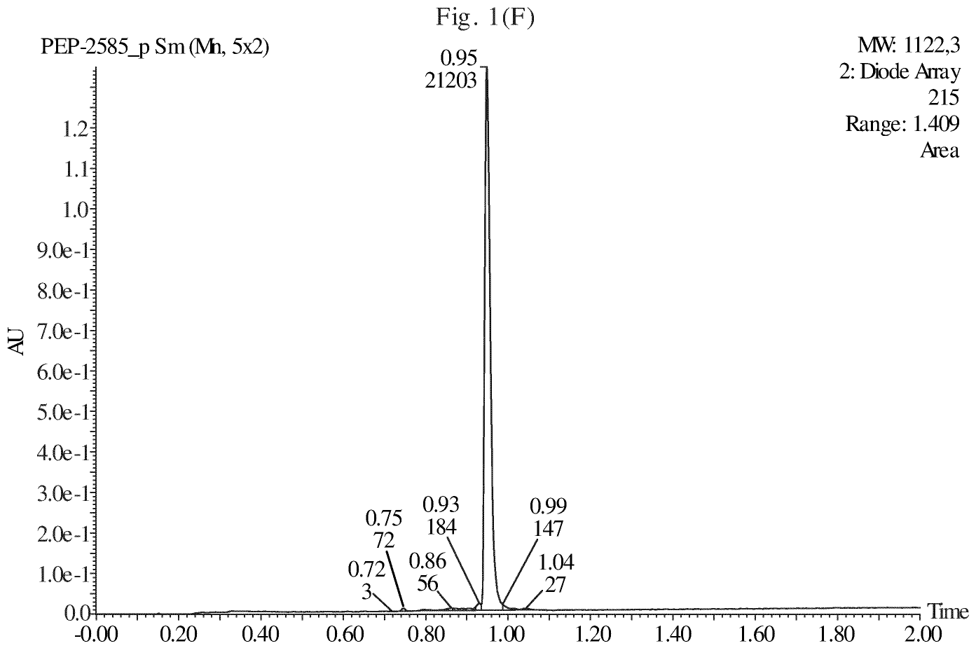

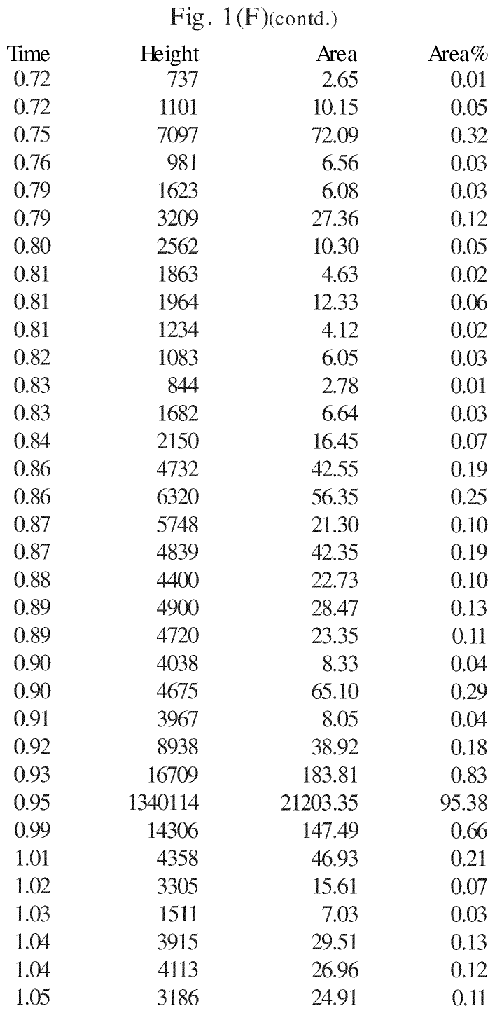

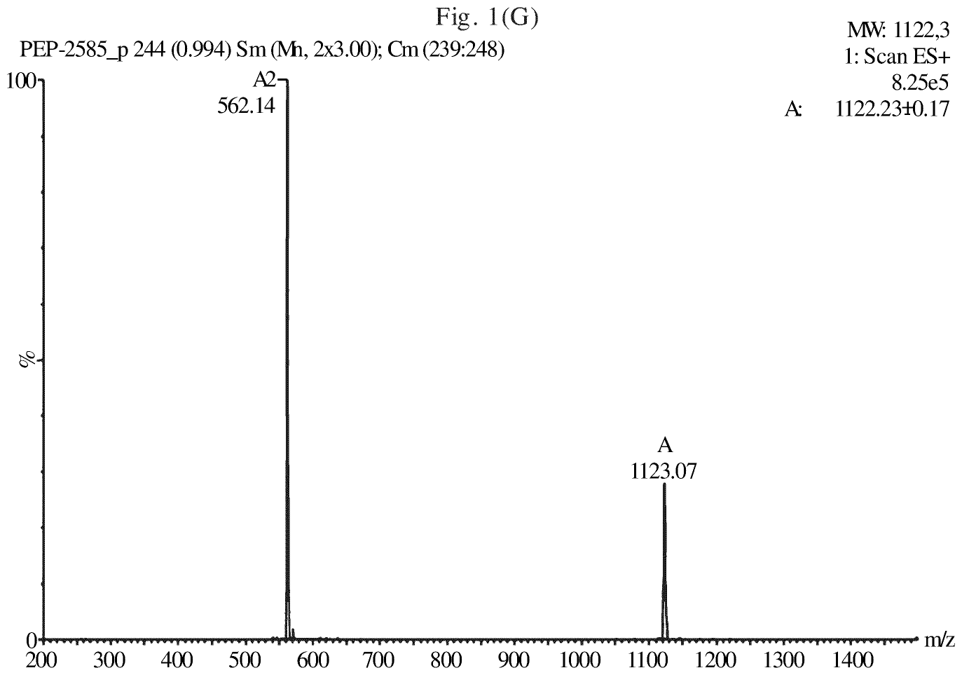

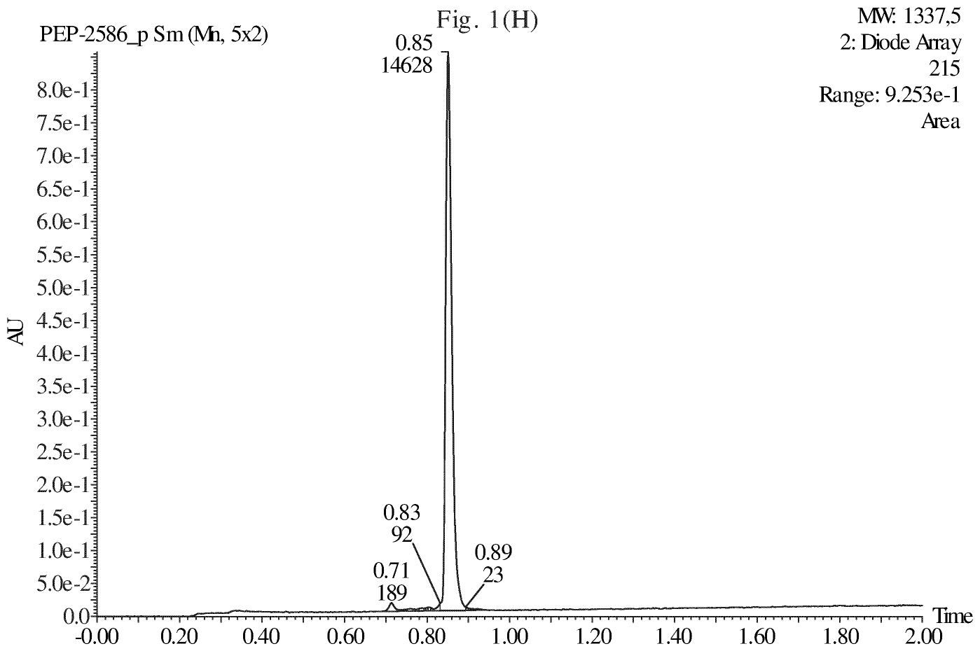

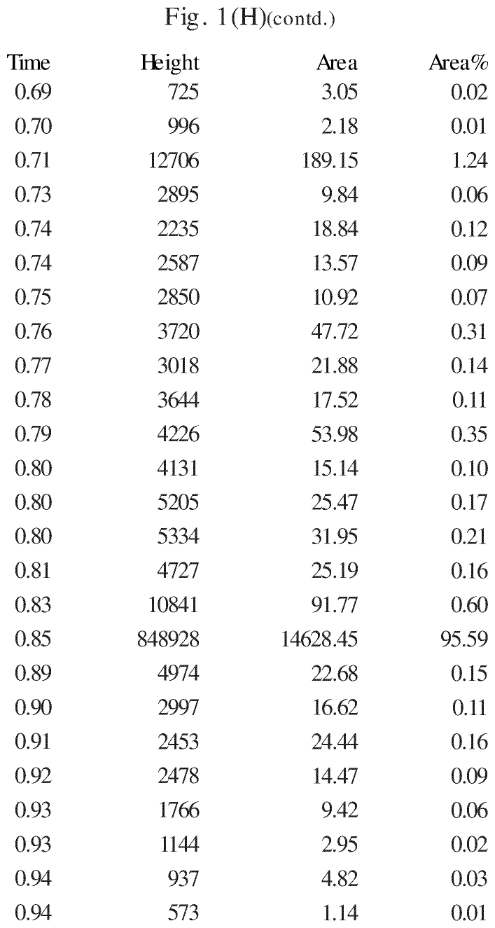

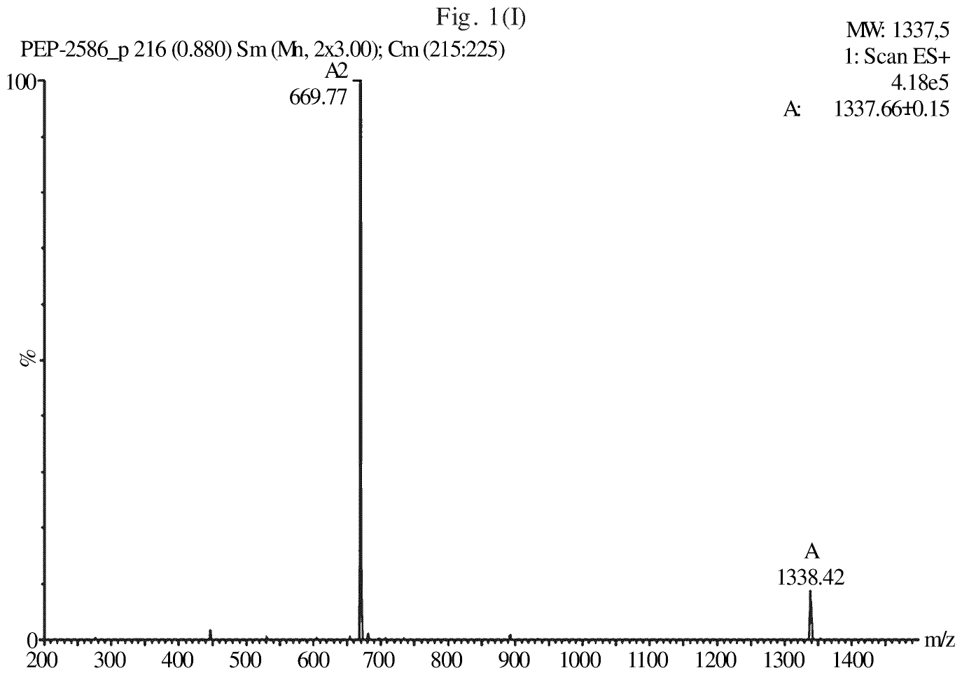

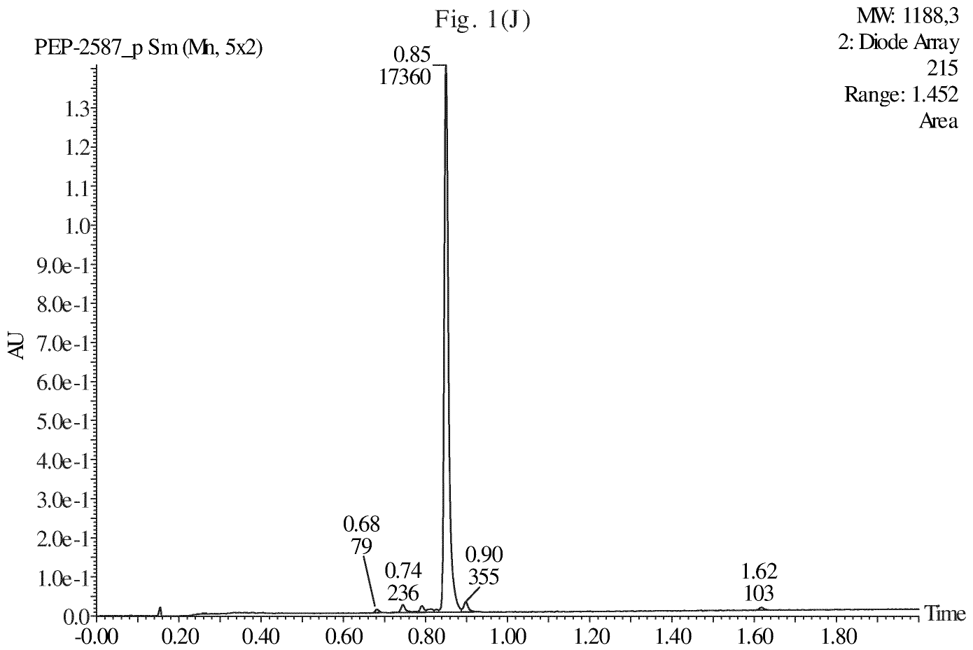

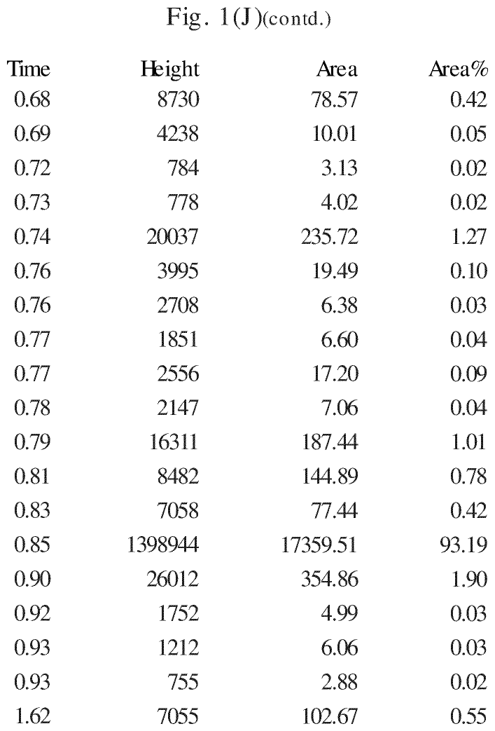

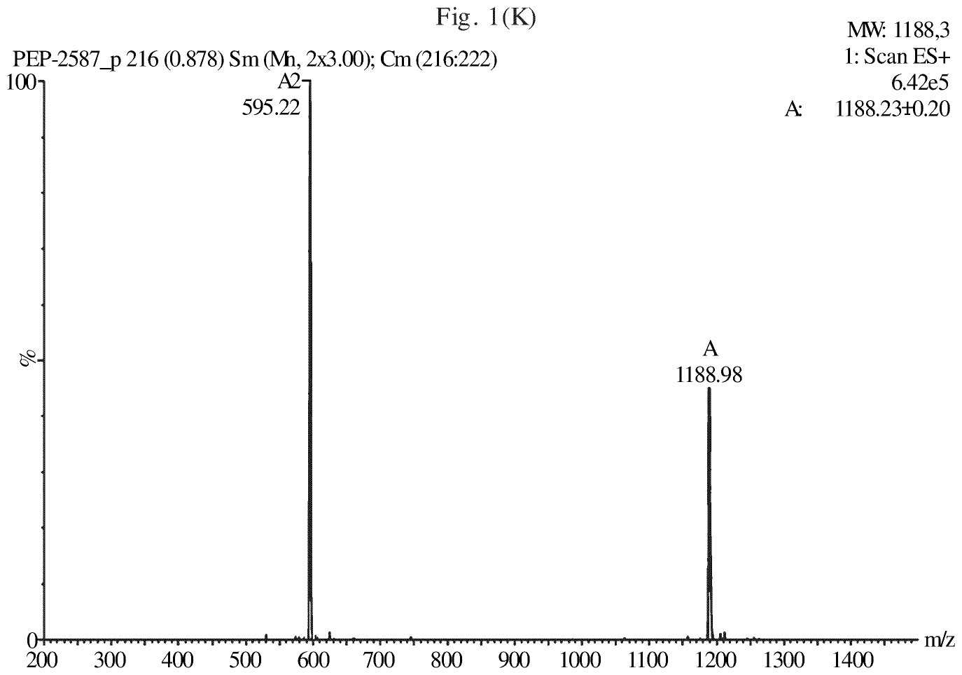

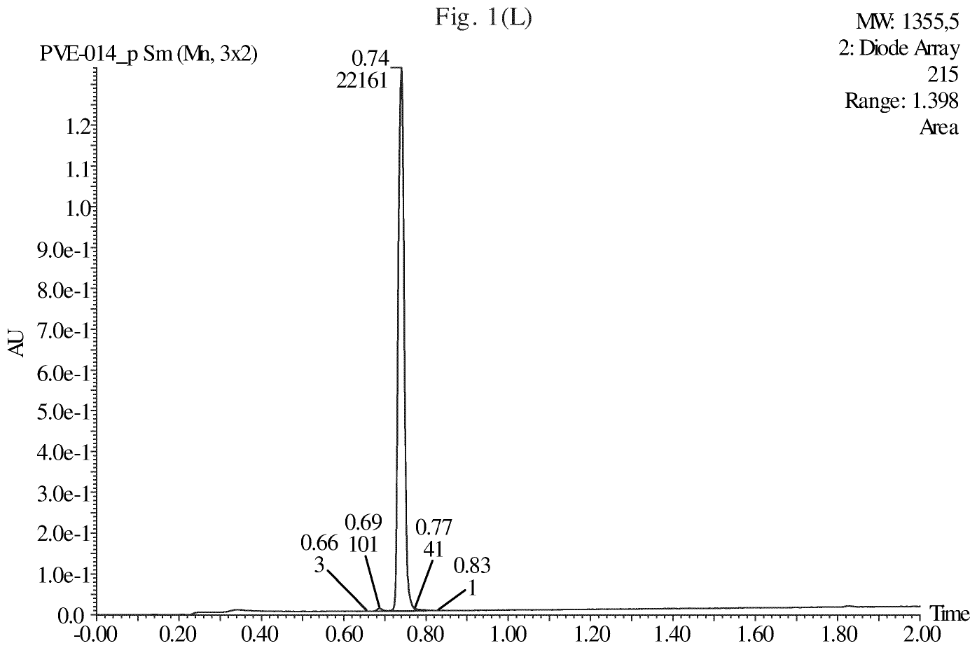

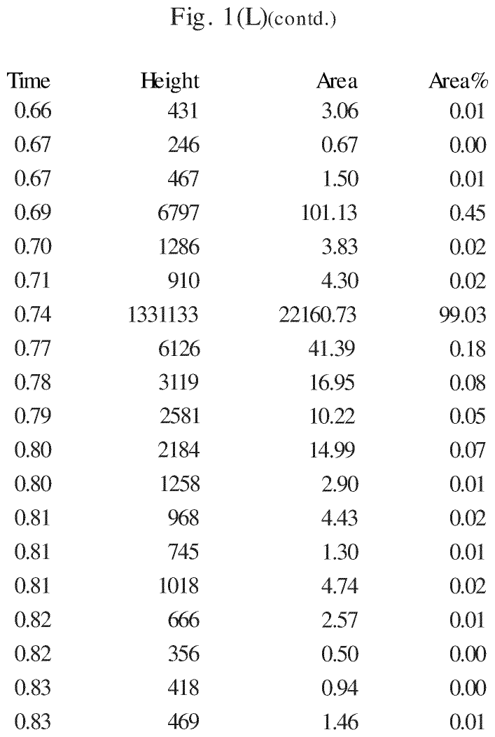

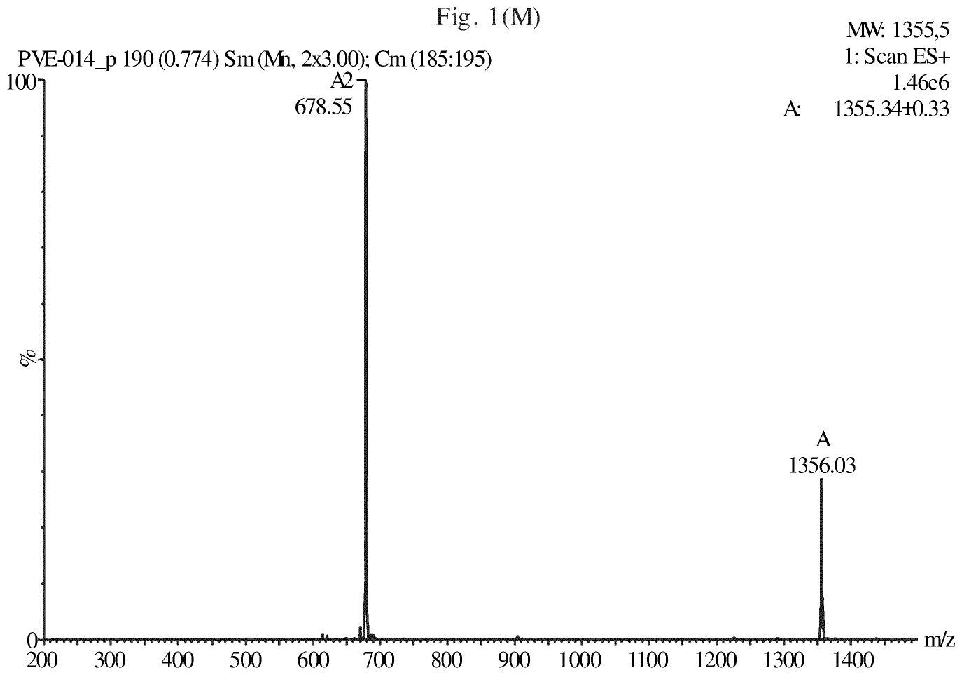

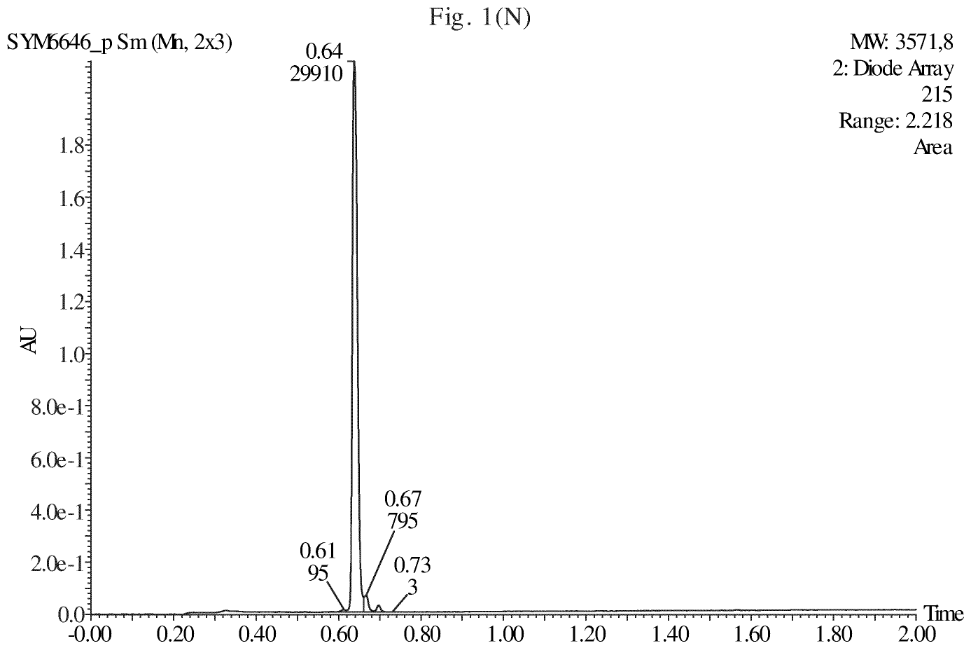

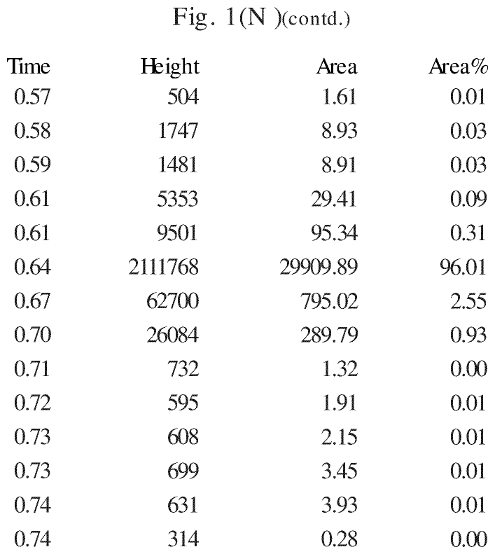

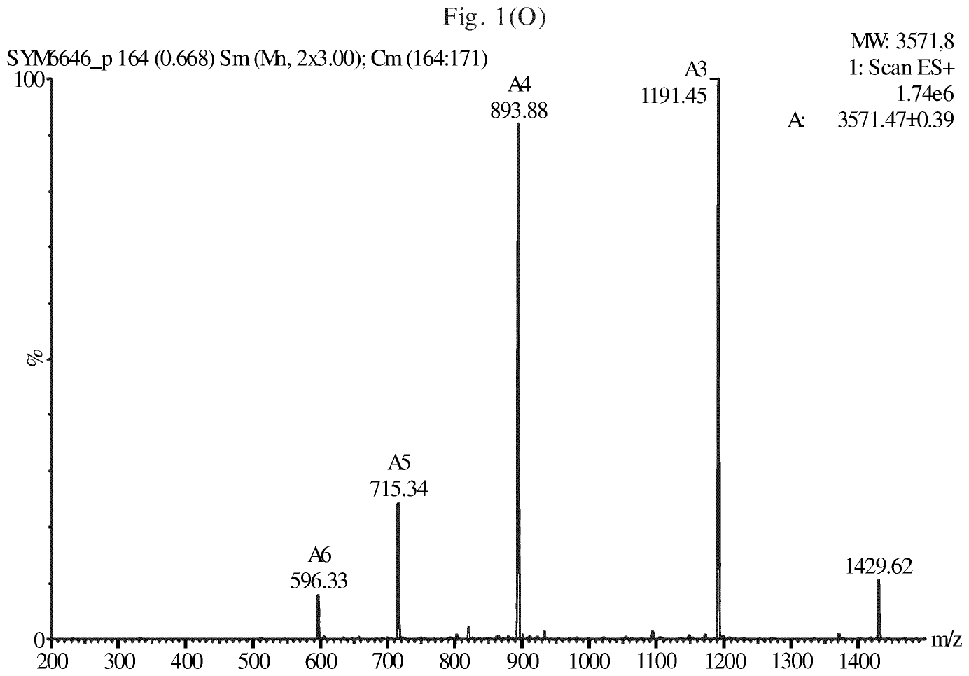

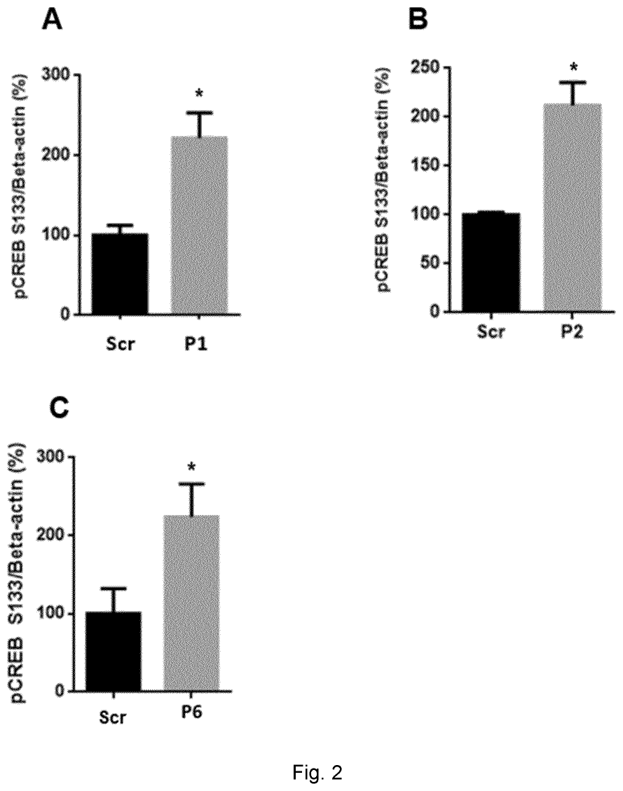

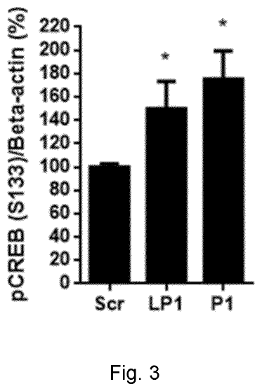

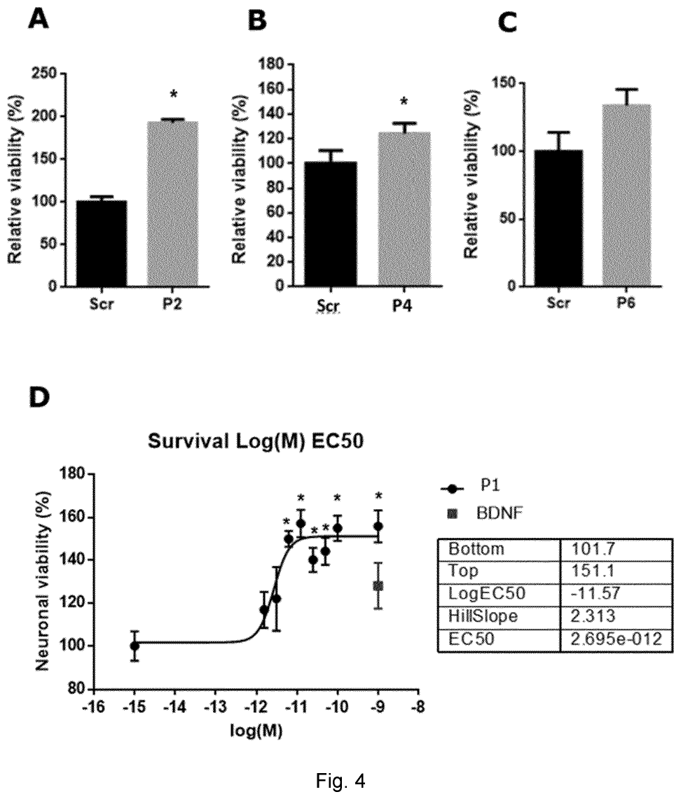

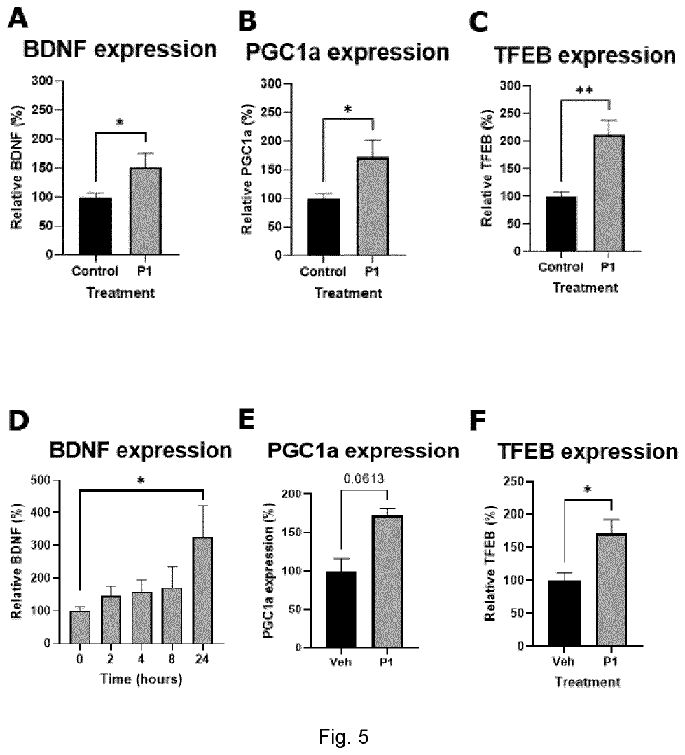

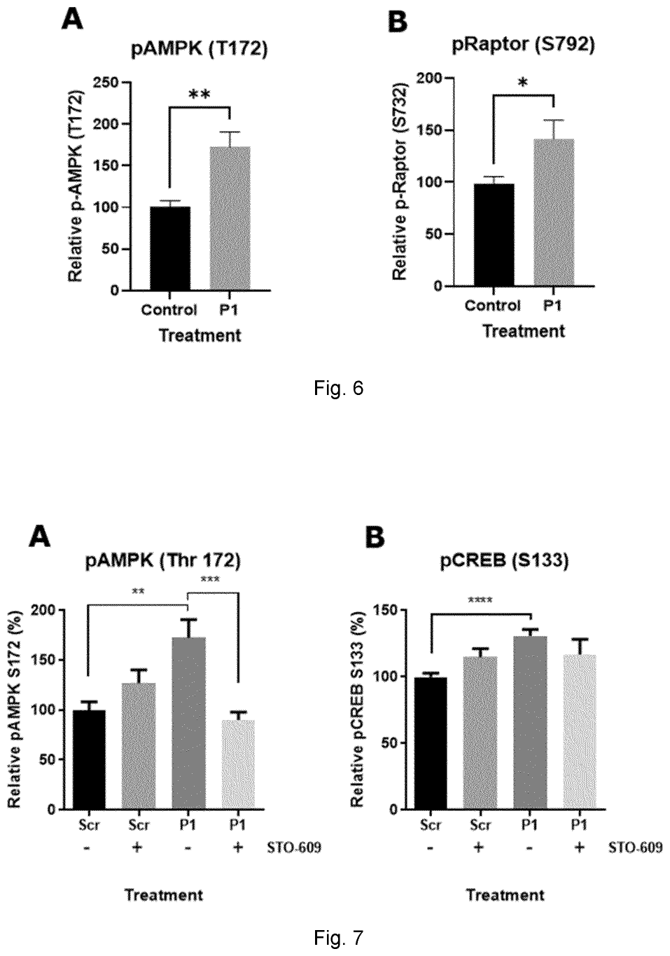

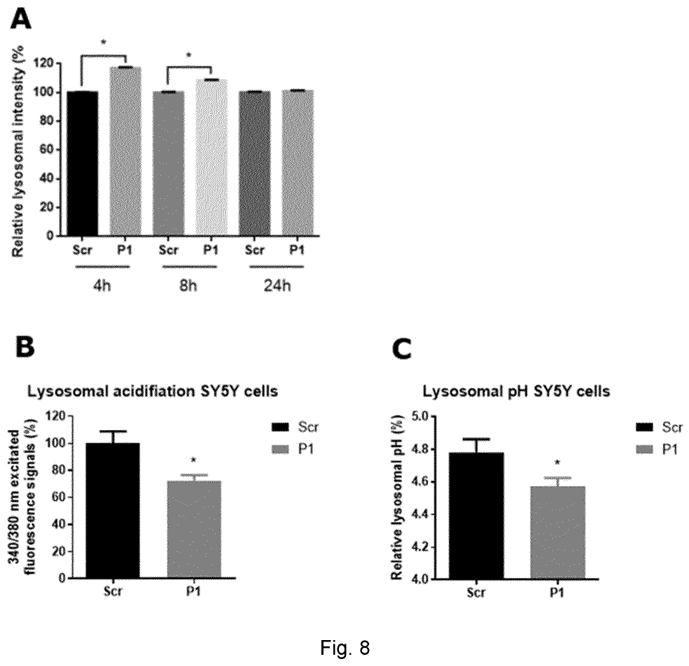

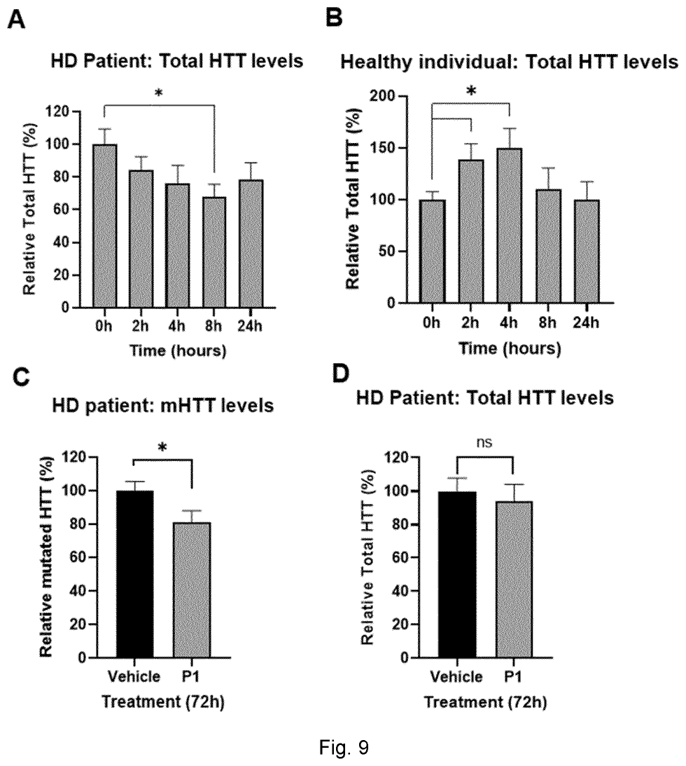

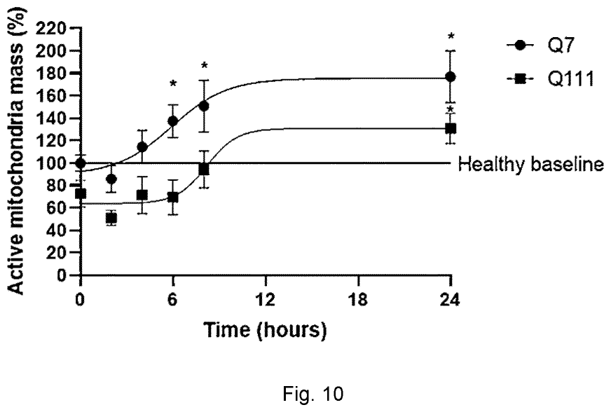

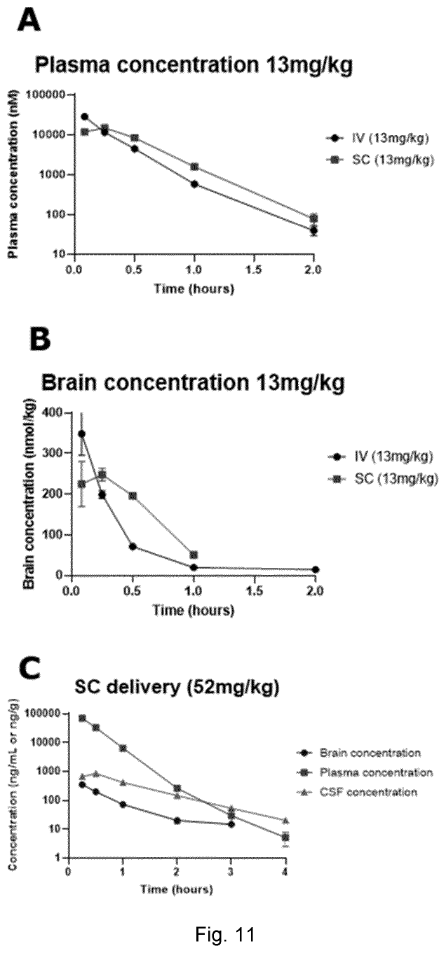

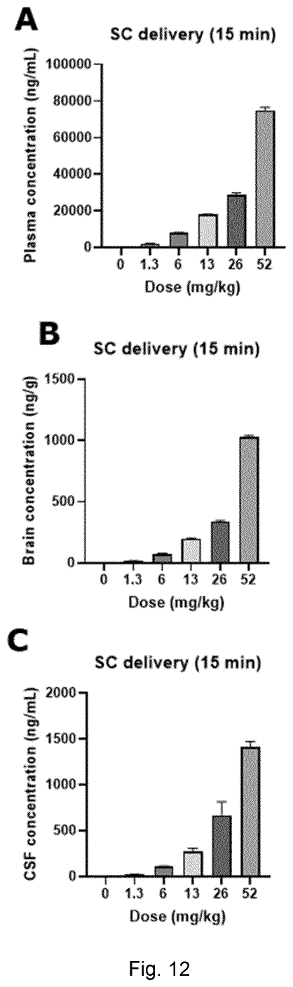

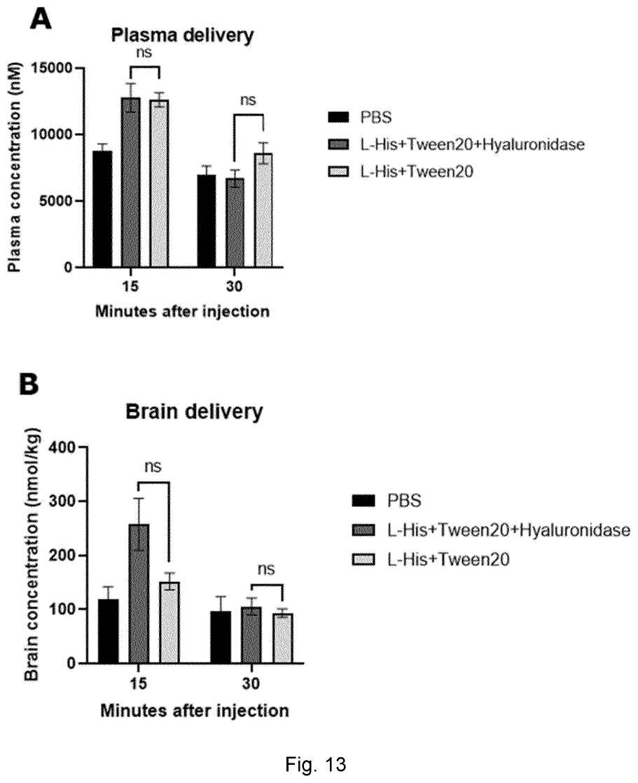

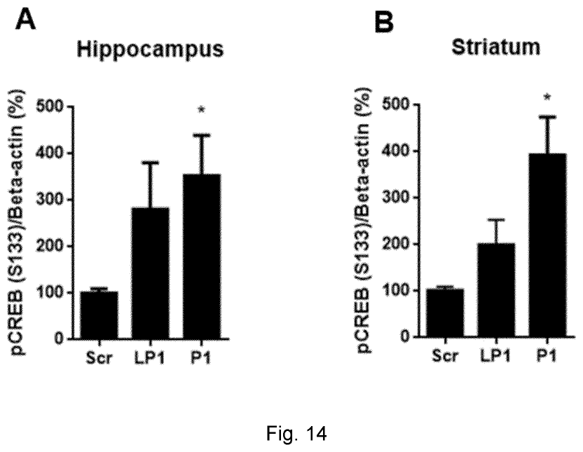

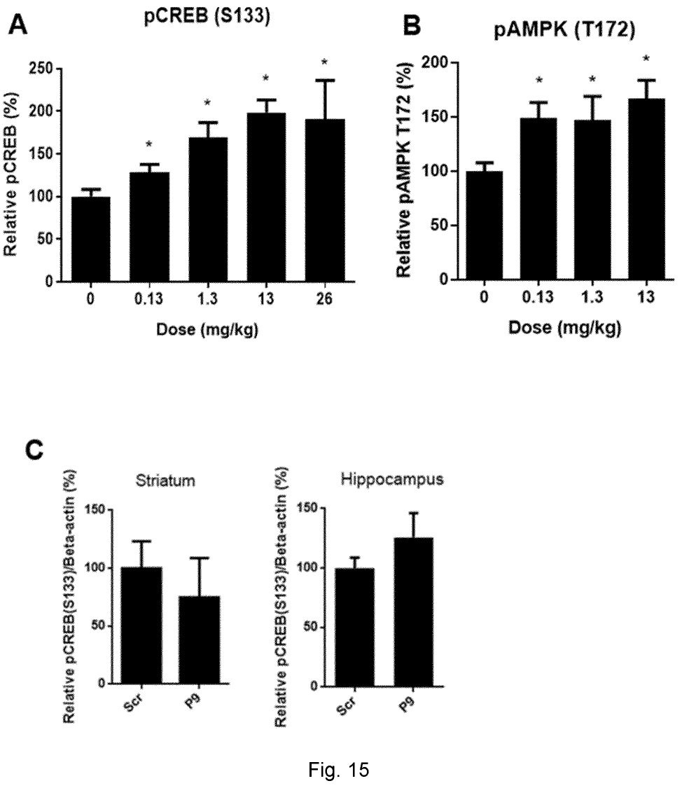

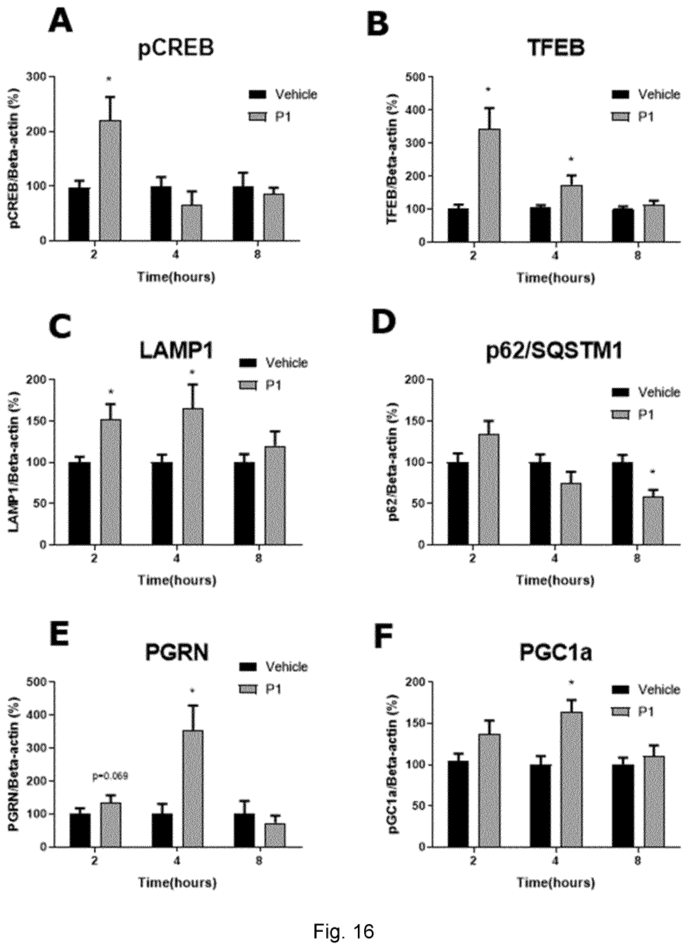

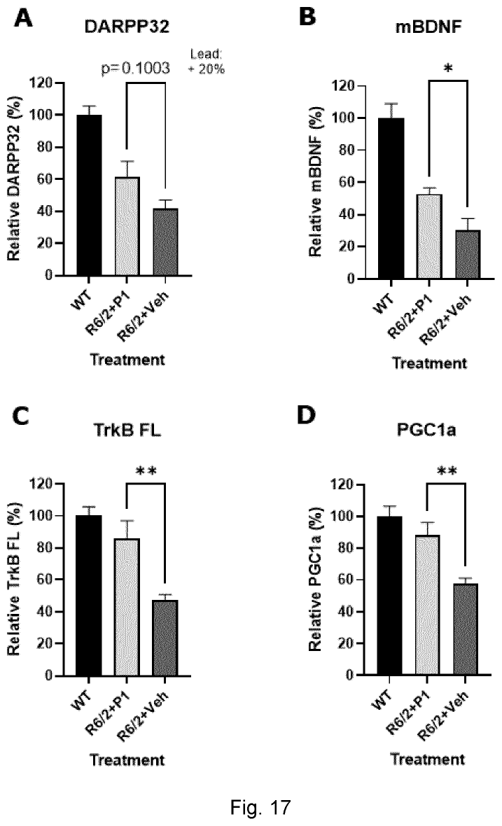

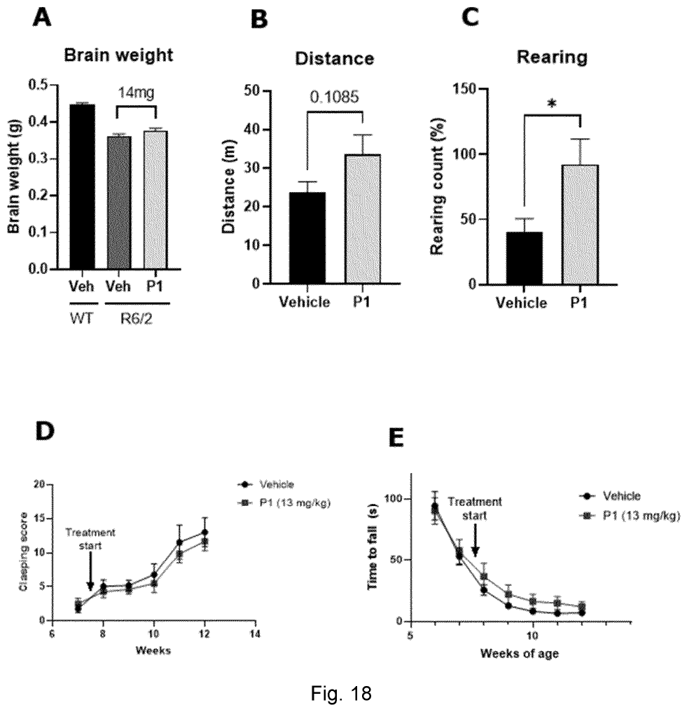

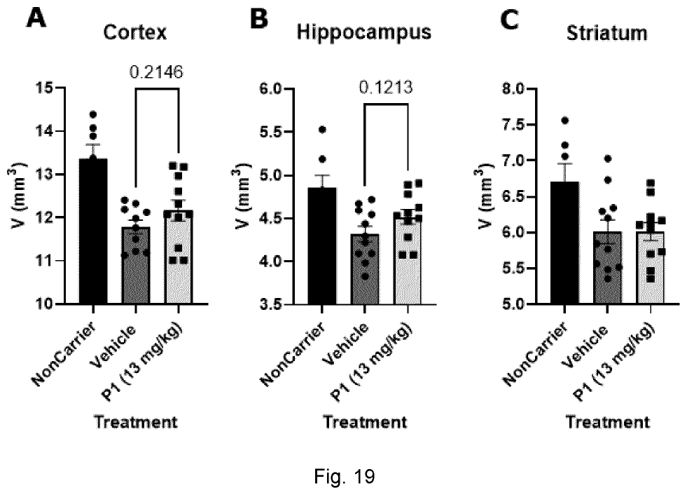

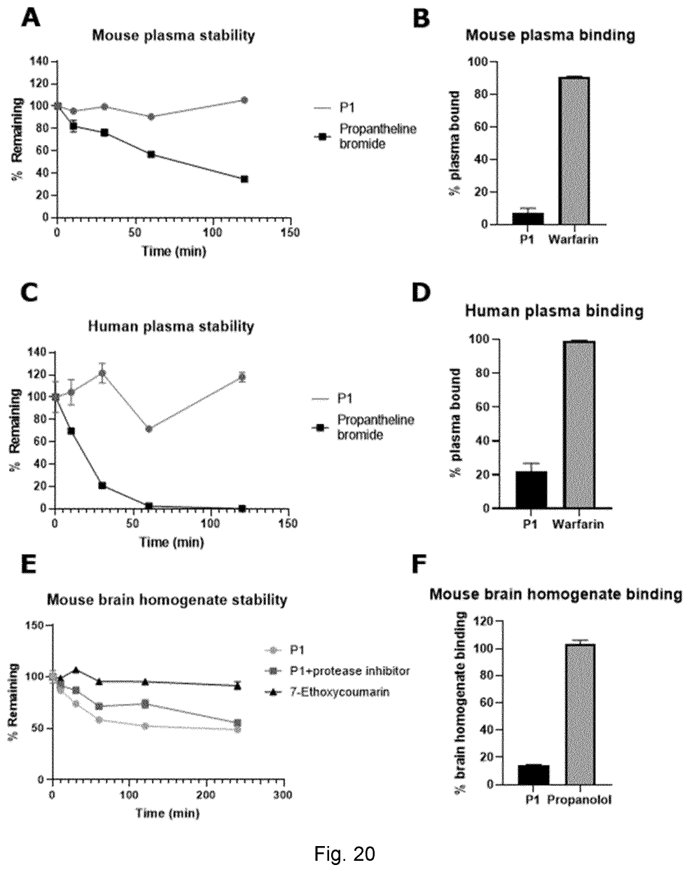

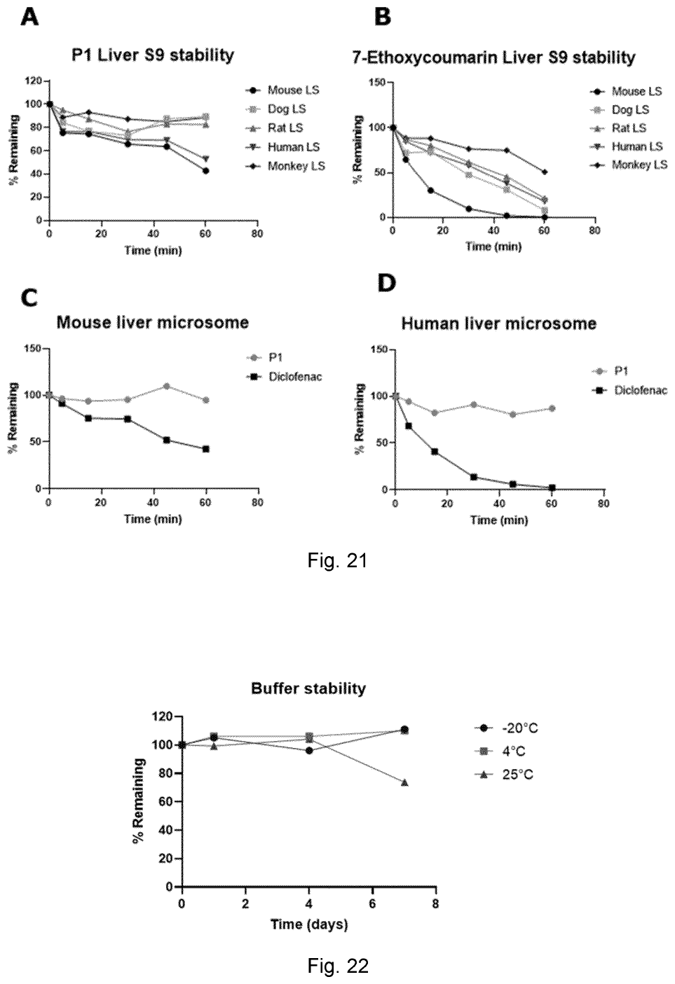

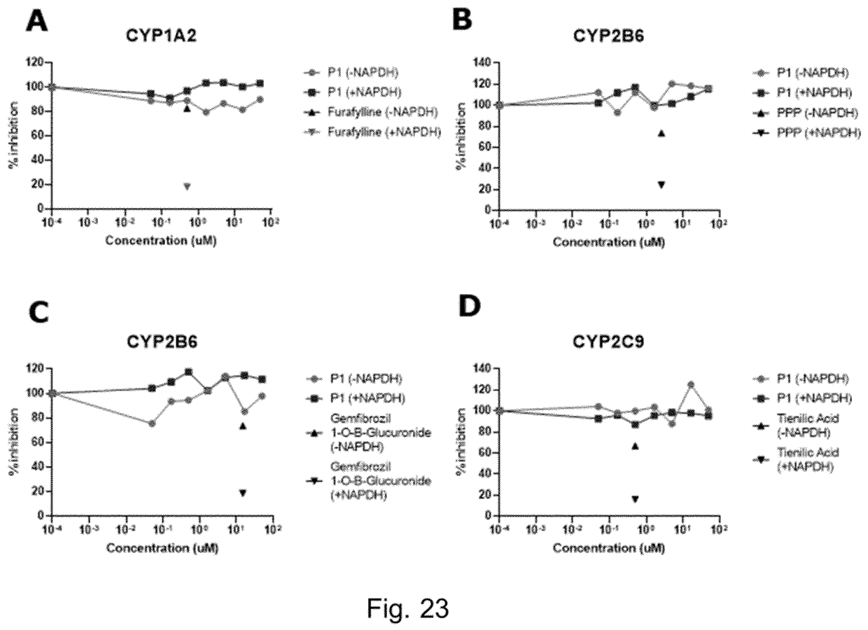

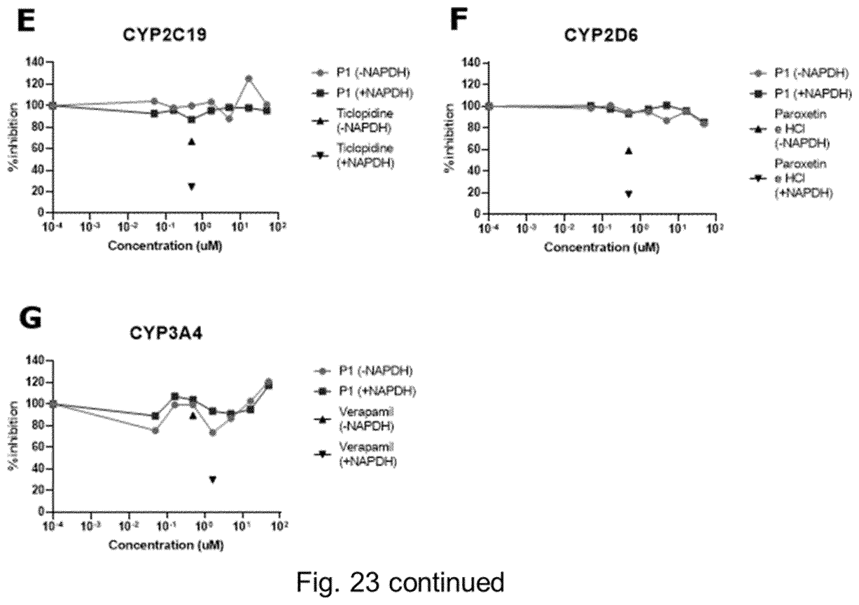

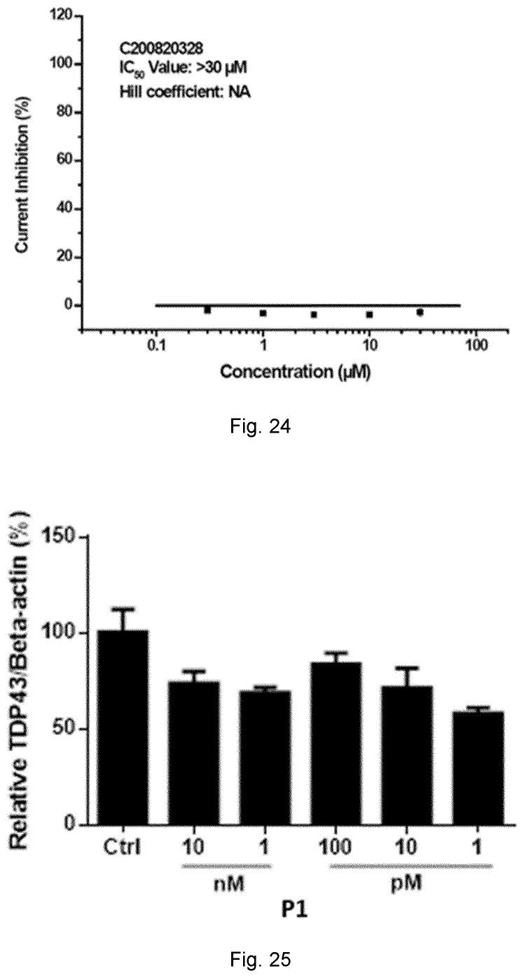

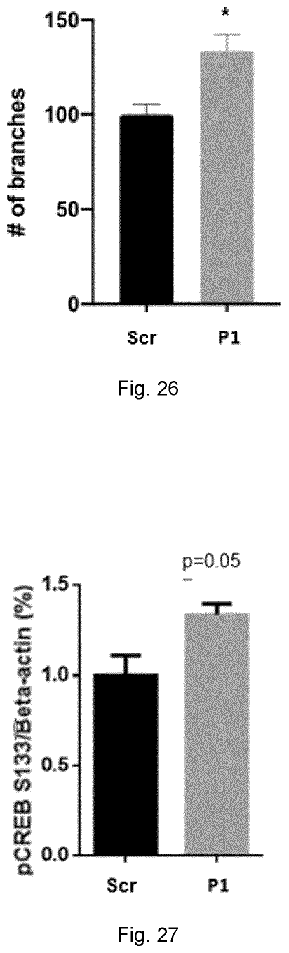

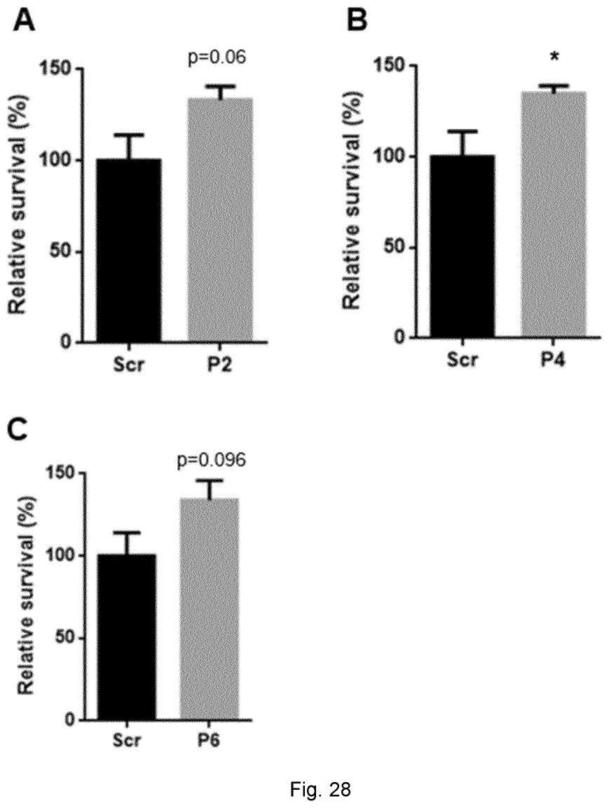

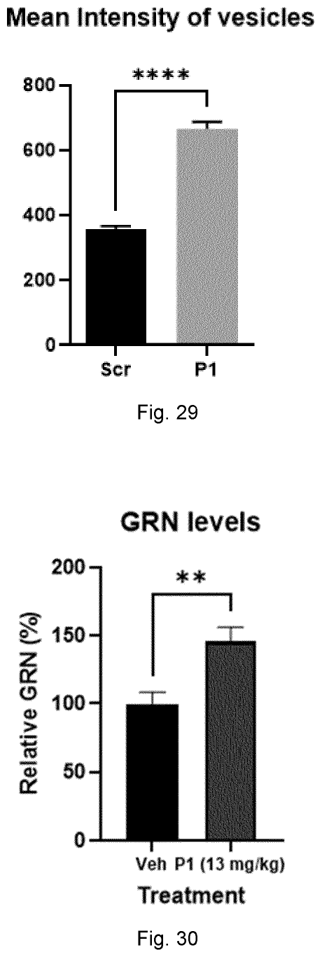

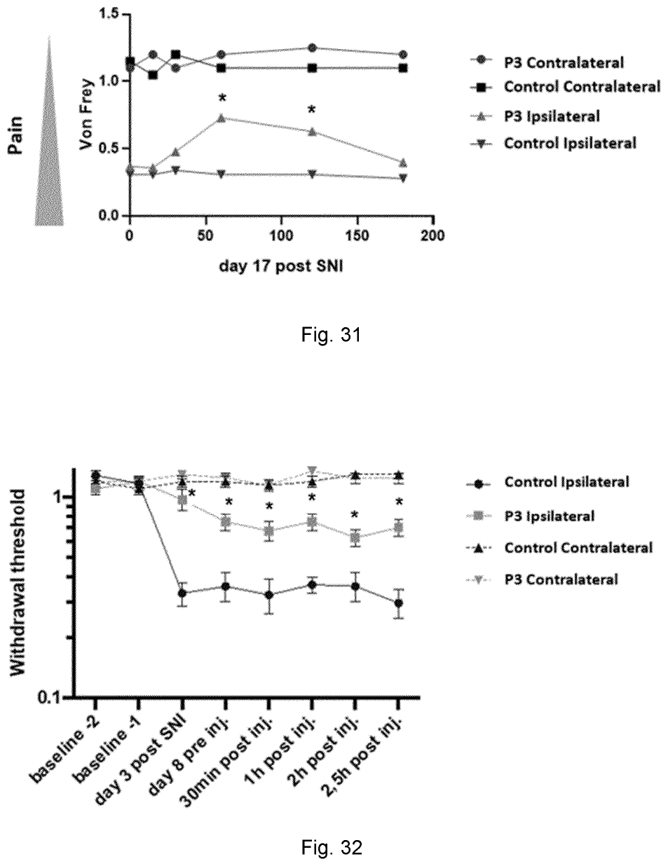

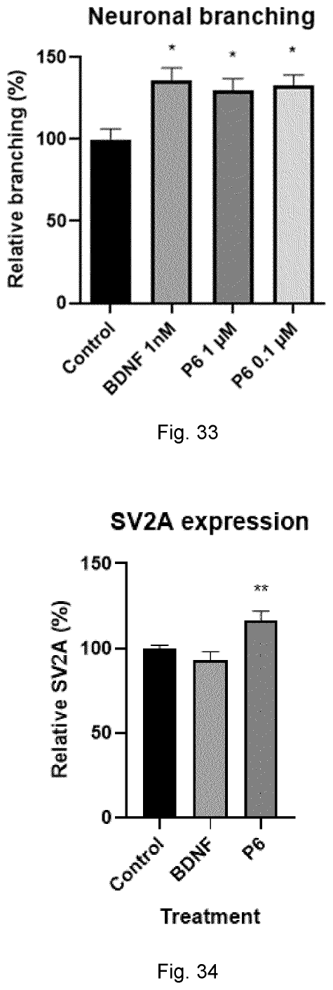

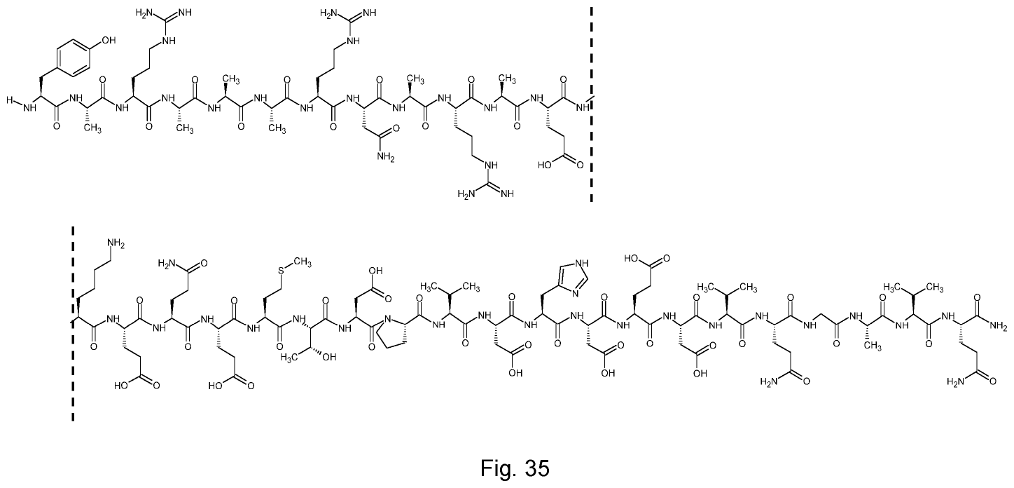

A qualitative analysis of the peptides was conducted by HPLC and LCMS. For example, see FIG. 1.

HPLC Method:
Column: Gemini C18, 110 Å, 5 μm, 150×4.6 mm
Gradient: Gradient run-time 20 minutes; 0.00-20.00 min 15-45% B. Column cleaning and equilibration; 20.00-20.10 45-95% B, 20.10-23.00 min 95% B, 23.00-23.10 min 95-15% B, 23.10-28.00 min 15% B
Flow rate: 1.0 mL/min
Diode array: 220/254 nm
Column temperature: 30° C.
Solvent A: 0.1% TFA in water
Solvent B: 0.075% TFA in acetonitrile

LC-MS Method for Final Products:
System: Agilent Infinity II 1260 HPLC series
Column: Xbridge C18, 130 Å, 3.5 μm, 2.1×30 mm
Detector: Agilent LCMS (G6125C), single quadrupole TIC scan
Scanning range: m/z min 100, m/z max. 2000, positive mode, electrospray
Gradient: Gradient run-time 1 minutes; 0.00-1.00 min 10-80% B. Column cleaning and equilibration; 1.00-1.01 80-95% B, 1.01-1.60 min 95% B, 1.60-1.61 min 95-10% B, 1.61-2.00 min 10% B
Flow rate: 1.2 mL/min
Diode array: 215 or 220 nm
Column temperature: Room temperature
Solvent A: 0.1% TFA in water
Solvent B: 0.075% TFA in acetonitrile

High resolution mass spectrometry (HMRS) was used to further confirm the identity of cyclic peptide P1 (a calibrated Agilent LCMS QTOF instrument using positive mode electrospray ionization)

Results

The purity and characterization of cyclic peptides P1 (SEQ ID NO: 1), P2 (SEQ ID NO: 2), P3 (SEQ ID NO: 3), P4 (SEQ ID NO: 4), P6 (SEQ ID NO: 6), linear peptide LP1 (SEQ ID NO: 8) and linear peptide P9 (SEQ ID NO: 9) are shown in FIG. 1A to FIG. 10.

Data on LC-MS and HPLC Purity is Shown in Table Below:

| | | | | Calc. | Calc. | Mass found | | | |
|---|---|---|---|---|---|---|---|---|---|
| | SEQ | HPLC | | monoiso- | average | m/z, | m/z, | m/z, | m/z, |
| Pep. | ID NO | purity | Sum formula | topic mass | mass | z = 1 | z = 2 | z = 3 | z = 4 |
| P1 | 1 | 99.7 | C₅₄H₈₁N₁₃O₂₂S | 1295.534 | 1296.3598 | 1296.7 | 649.1 | — | — |
| P2 | 2 | 96.2 | C₅₁H₇₅N₁₃O₂₂S | 1253.487 | 1254.2801 | 1254.76 | 627.98 | — | — |
| P3 | 3 | 95.8 | C₄₈H₇₅N₁₃O₁₆S | 1122.218 | 1122.2516 | 1123.07 | 562.14 | — | — |
| P4 | 4 | 95.6 | C₅₆H₈₈N₁₆O₂₀S | 1336.608 | 1337.4581 | 1338.42 | 669.77 | — | — |
| P6 | 6 | 93.2 | C₄₈H₇₇N₁₃O₂₀S | 1187.513 | 1188.2651 | 1188.98 | 595.22 | — | — |
| P8 | 8 | 99.0 | C₅₆H₈₆N₁₄O₂₃S | 1354.571 | 1355.4270 | 1356.03 | 678.55 | — | — |
| P9 | 9 | 96.0 | C₁₄₆H₂₃₃N₄₉O₅₄S | 3568.671 | 3570.7731 | — | — | 1191.45 | 893.88 |

HRMS:

| | SEQ | | Calc. mass | Mass found | Mass found | |
|---|---|---|---|---|---|---|
| Peptide | ID NO | Sum formula | m/z, z = 1 | m/z, z = 1 | m/z, z = 2 | Mass found |
| P1 | 1 | C₅₄H₈₁N₁₃O₂₂S | 1296.5418 | 1296.5406 | 648.7768 | 1295.5365 |

### Example 3: Effect on Transcription Factor CREB Activation

To assess the ability of peptides P1, P2 and P6, respectively, to activate transcription factor CREB, cortical neurons were isolated from p0 wild-type mice and seeded in a density of 500.000 per well. After 7 days in vitro the neurons were stimulated with the different peptide variants at 1 uM in neurobasal A media and incubated at 37° C. and 5% C02 for 20 minutes. Hereafter, the neurons were lysed in lysis buffer containing DTT and cOmplete cocktail protease inhibitor and subsequently sonicated to disrupt the nuclear membrane. The phosphorylation of CREB on serine 133 was validated by western blotting normalized to beta-actin. As control, cells were stimulated with a scrambled peptide (Scr). Student's t-test was used for statistical analysis.

As seen in FIG. 2A to FIG. 2C, each of the peptides P1, P2 and P6 markedly activated CREB compared to scrambled peptide (p<0.05).

### Example 4: A Comparative Study on the Effect on CREB Activity

The ability of peptide P1 and a linear peptide analog of P1 with acetylated N- and amidated C-terminals (referred to as LP1) to activate transcription factor CREB was assessed. Cortical neurons were isolated from p0 wild-type mice and seeded in a density of 500.000 per well. After 7 days in vitro the neurons were stimulated with the different peptide variants at 20 nM in neurobasal A media and incubated at 37° C. and 5% CO₂ for 20 minutes. Hereafter, the neurons were lysed in lysis buffer containing DTT and cOmplete cocktail protease inhibitor and subsequently sonicated to disrupt the nuclear membrane. The phosphorylation of CREB on serine 133 was validated by western blotting normalized to beta-actin. As control, cells were stimulated with a scrambled peptide (Scr). Student's t-test was used for statistical analysis.

As seen in FIG. 3, both peptide P1 and LP1 activated CREB compared to scrambled peptide (p<0.05) whereas LP1 activated CREB to a lesser extent, demonstrating an increased efficacy of the cyclic peptide P1 compared to the related linear version LP1.

### Example 5: Peptides P1, P2, P4 and P6 Increase Survival in Cortical Neurons

One consequence of CREB-activation is the upregulation of pro-survival genes leading to decreased apoptotic signalling and increased neuroprotection^{2,38}. It was thus investigated if treating cortical neurons would increase their ability to survive. This was assessed in primary neuronal cultures, as these spontaneously disintegrate and die as they get older in vitro.

Cortical neurons were isolated from p0 wild-type mice and seeded in a density of 50.000 in a 96-well plate pre-coated with poly-L and laminin and incubated at 37° C. in a 5% C02 atmosphere. At 7 days in vitro (DIV7) the neurons were treated by changing half the media with neurobasal A with B27 containing the peptides (P2, P4 or P6) to give a finale concentration of 1 uM and further incubated. The cortical neurons were treated in a likewise manner on DIV9 and DIV11. At DIV12 half the media was removed and 3-(4,5-dimethylthiazole-2-yl)-2,5-diphenyltetrazolium bromide (MTT) was added to the media to give a finale concentration of 0.5 mg/mL. MTT is reduced by mitochondrial enzymes in active cells to an insoluble product called formazan, which can be detected by colorimetry. Thus, the colour generated reflects activity and viable cells in the culture. The neurons were incubated for 4 hours with MTT and thereafter lysed in a 50% EtOH and 50% DMSO solution. To assure proper mixing of formazan, the plate was left to shake for 30 minutes before being measured at 570 nm and 650 nm. Student's t-test was used for statistical analysis.

The results are depicted in FIG. 4A to FIG. 4C as the relative survival compared to control (neurons treated with a scrambled peptide). Peptides P2, P4 and P6 increased the relative survival compared to non-treated.

Further, a drug-dose response on survival for peptide P1 was assessed in a similar manner. The results (FIG. 4D) indicate, that peptide P1 increases survival with an EC50 value of 2.7 μM. BDNF was used as a positive control, which increased survival to a lesser extent than peptide P1.

In conclusion, treatment with peptides P1, P2, P4 and P6 caused an increase in survival of cortical neurons.

### Example 6: Peptides P1 Treatment Leads to Increase in CREB-Targeted Genes

Activation of CREB is well-known to improve survival, synaptic formation and growth in neurons. A critical mediator of this is the production of the neurotrophic factor, BDNF, by CREB³⁹. Similarly, CREB activation has been described to induce both lysosomal- and mitochondrial biogenesis through the production of the two master regulators TFEB and PGC1a, respectively⁴⁰⁻⁴². We therefore assessed whether P1 treatment leads to increased BDNF, TFEB and PGC1a production in wild-type neurons as a consequence of CREB activation.

Cortical neurons were isolated from p0 wild-type mice and seeded in a density of 200,000 per well (24-well tray). After 7 days in vitro the neurons were stimulated with 1 uM P1 in neurobasal A media and incubated at 37° C. and 5% CO₂ for 4 hours or 6 hours. Hereafter, the neurons were lysed in RIPA lysis buffer containing cOmplete cocktail protease inhibitor.

As shown in FIG. 5A to FIG. 5C, P1 upregulated all of the assessed CREB-downstream target genes of BDNF, TFEB and PGC1.

To validate the therapeutic potential of P1 in Huntington's Disease, we likewise assessed whether P1 could increase the three distinct pathways in fibroblasts derived from a HD patient. The fibroblasts were bought from Coriell Biobank. The patient-derived fibroblasts, named GM04719, was sampled from a 39-year-old female with clinical onset at 46 years of age. The patient has 44-CAG repeats in their HTT-gene.

To assess target engagement by P1, 30,000-50,000 fibroblasts were seeded per well in a 96-well plate. The following day, the cells were treated with 1 uM of P1 and incubated at 37° C. and 5% CO₂ at timepoints between 2-24 hours. Hereafter, the cells were lysed in RIPA lysis buffer containing cOmplete cocktail protease inhibitor. BDNF, PGC1a and TFEB levels were analysed by western blotting normalized to beta-actin.

FIG. 5D to FIG. 5F show that BDNF is significantly upregulated after 24 hours, while TFEB and PGC1a is upregulated at 6 hours of treatment. This shows target engagement in this HD cell line.

This demonstrates that P1 might influence both pro-survival effects through BDNF upregulation, lysosomal processes through TFEB upregulation and mitochondrial functions through PGC1a upregulation in both healthy and HD cells suggesting a therapeutic approach as these functions are all impaired in HD patients. Student's t-test was used for statistical analysis.

### Example 7: Activation of Lysosomal Pathways

Previous studies demonstrated that master regulator of lysosomal biogenesis, TFEB, was upregulated. To further validate an activation of these lysosomes, we assessed lysosomal-pathway activation following P1-treatment (SEQ ID NO: 1). AMPK is a well-described activator of lysosomes through its inhibition of the mTOR complex (a complex which inhibits lysosome acidification) and further activation of TFEB⁴⁶. AMPK is activated on threonine 172⁴⁷ and activated phospho-AMPK subsequently phosphorylates Raptor (component of mTOR complex) on serine 792, which inactivates the mTOR complex⁴⁸.

To assess the ability of peptide P1 to activate AMPK and subsequently inactivate the mTOR complex, mouse cortical neurons were isolated from p0 wild-type mice and seeded in a density of 250,000 per well in a 12-well tray. After 7 days in vitro the neurons were stimulated with P1 in neurobasal A media and incubated at 37° C. and 5% CO₂ for 20 minutes. Hereafter, the neurons were lysed in lysis buffer containing DTT and cOmplete cocktail protease inhibitor. The phosphorylation of AMPK (T172) and Raptor (S792) was validated by western blotting normalized to beta-actin.

FIG. 6 shows that P1 both activates AMPK by phosphorylation on T172 (A) and further inactivates Raptor by phosphorylation on S792 (B). This demonstrates that P1 not only increases lysosomal biogenesis, but additionally activates lysosomes through inhibition of the mTOR complex. Student's t-test was used for statistical analysis.

### Example 8: P1 Activation of AMPK and CREB is Blocked by STO-609 (CaMKK2-Inhibitor)

To further understand the mechanism by which P1 (SEQ ID NO: 1) activates AMPK and CREB we assessed whether CaMKK2 was involved. CaMKK2 has previously been shown to directly activate AMPK (T172) and additionally activate CREB (S133), through CAMK4⁴⁹⁻⁵¹. To assess the involvement of CaMKK2 in the mechanistic function of P1, a commercially available selective potent inhibitor of CaMKK2 was used (STO-609).

Mouse cortical neurons were isolated from p0 wild-type mice and seeded in a density of 250,000 per well in a 12-well tray. After 7 days in vitro the neurons were either pre-treated with STO-609 (5 uM) for 1 hour or with DMSO (control). After 1 hour, the neurons were stimulated with P1 or a scrambled peptide (Scr), with or without STO-609, in neurobasal A media and incubated at 37° C. and 5% CO₂ for 20 minutes. Hereafter, the neurons were lysed in lysis buffer containing DTT and cOmplete cocktail protease inhibitor. Phosphorylation of AMPK (T172) and CREB (S133) was validated by western blotting and normalized to beta-actin.

FIG. 7 shows inhibition of AMPK (A) following STO-609-treatment and moreover loss of P1-induced activation of AMPK, indicating that CaMKK2 is involved for this process. Furthermore, CREB activation by P1 was lowered but not totally abolished in neurons pre-treated with STO-609 compared to P1-treated cells only, which suggests a partial role of CaMKK2 in this process. Student's t-test was used for statistical analysis.

### Example 9: Peptide P1 Increases Lysosomal Acidification

An important aspect in targeting neurodegenerative diseases is the clearance of toxic aggregates—a hallmark in these diseases. These aggregates are mainly cleared by either the lysosomal-autophagic network or through proteasomal degradation⁵². To determine the therapeutic potential of peptide P1, the direct acidification of lysosomes (a measurement of lysosomal activation) was assessed, which CREB, TFEB and AMPK have previously been demonstrated to affect^{41,43,44,46}. One way to assess this is to study the acidification of lysosomes, as this is a direct measurement of lysosomal activity and therefore can be used as a readout for degradation. Induction of lysosomal acidification was carried out in both primary hippocampal neurons and in SH-SY5Y cells (human neuroblastoma cell line).

Primary hippocampal neurons were seeded in an 8-well ibidi chamber pre-coated with Poly-I and laminin. 75.000 neurons were seeded pr. well. After 5 days in vitro the neurons were stimulated for 4, 8 or 24 hours with peptide P1 at 1 uM in neurobasal A with B27. After stimulation, the medium was changed to pre-warmed (37° C.) neurobasal A containing 1 uM of LysoSensor probe DND-189 (an acidotropic probes that accumulates in acidic organelles as the result of protonation) and incubated for 30 min. at 37° C. in a 5% C02 atmosphere. Hereafter, the media was changed to pre-warmed FluoroBrite Medium and the neurons were imaged in Olympus microscopy system. Pictures were taken of the lysosomes and processed using the ScanR imaging software to give fluorescence (intensity) of each lysosomal vesicle. The summed total intensity of all lysosomes was calculated and divided by the total number of neurons imaged yielding the total intensity of lysosomes per neuron. Student's t-test was used for statistical analysis.

SH-SY5Y were seeded in poly-L coated black 96 well plate with clear bottom at a density of ˜3×10⁴. The following day, the cells were stimulated with 1 uM P1 (SEQ ID NO: 1) for 4 hours and subsequently incubated with LysoSensor™ Yellow/Blue DND-160 (10 uM) for 20 min. Cells were rinsed with PBS and excitation 340 & 380 and emission 535 was read in a plate-reader. A pH calibration curve buffer was likewise measured. Calculation of the fluorescence intensity ratio of excitation (340/380) results in an average whole-cell intraorganellar pH reflective of all LysoSensor™ Yellow/Blue DND-160-labeled organelles combined. Using the generated linear trendline from the standard curve for pH and 340/380 intensity ratio, calculate the intraorganellar pH of samples.

The results are shown in FIG. 8. After 4 and 8 hours of treatment, the lysosomal intensity was increased in P1 treated neurons, demonstrating an increase in the acidification of lysosomes. However, after 24 hours no difference between control and P1-treated neurons was observed. This demonstrates that peptide P1 rapidly increases lysosomal activity in wild-type neurons, which declines before 24 hours (FIG. 8A). Similarly, an increase in lysosomal acidification was observed in SH-SY5Y cells after 4 hours of stimulation (FIG. 8B), shown by a reduction in the 340/380 intensity ratio. This corresponded to a drop of ˜0.2 pH in lysosomes (FIG. 8C).

In conclusion, treatment with peptide P1 caused an increase in lysosomal acidification, which is considered one of the important mechanisms that clear misfolded and toxic aggregates present in neurodegenerative diseases. Hence, these results indicate that peptide P1 is useful in the treatment of neurodegenerative diseases and lysomal storage disorders.

### Example 10: P1 Clears Soluble mHTT in Huntington's Patient-Derived Fibroblasts

Autophagy is the process of clearing misfolded proteins, aggregates or damaged organelles. As P1 (SEQ ID NO: 1) activates lysosomal pathways and furthermore shows target engagement in HD patient-derived fibroblasts (see Example 6), we validated whether P1 could decrease the disease-causing gene in HD, HTT.

GM04719 was seeded at 30,000-50,000 per well in a 96-well. The following day, the cells were treated with 1 uM of P1 and incubated at 37° C. and 5% CO₂ at timepoints 0-24 hours. Hereafter, the cells were lysed in RIPA lysis buffer containing cOmplete cocktail protease inhibitor. Total Huntingtin levels were analysed using antibody mab2166 (Sigma-Aldrich) by western blotting and normalized to beta-actin. To determine, whether P1 also clears the healthy allele, we further assessed levels of total HTT in fibroblast derived from a healthy individual (GM01650E), bought from Coriell Biobank.

FIG. 9A and FIG. 9B demonstrate that P1 (SEQ ID NO: 1) reduces total HTT levels in patient-derived fibroblasts at 8-hours of stimulation (A), while increasing the total HTT levels in the healthy cell line (B). This indirectly indicates, that P1 selectively targets the disease allele for degradation, while not affecting the healthy allele.

To get a direct measurement and further validation of the selective degradation of the disease-allele another study was carried out in GM04719 fibroblasts. Here, at 30,000-50,000 were seeded per well in a 96-well. The following 3 days, the cells were treated with 1 uM of P1 every 24 hour and during this incubated at 37° C. and 5% CO₂. 24 hours after third treatment, the cells were lysed in RIPA lysis buffer containing cOmplete cocktail protease inhibitor. Mutated Huntingtin levels were measured using antibody, which only detects the disease-allele (MW1 ab). Total Huntingtin levels were likewise analysed using antibody mab2166 and both were normalized to beta-actin.

FIG. 9C and FIG. 9D demonstrate, that P1 treatment of patient-derived fibroblast reduced the toxic disease-allele by 20% while not lowering the total amount of Huntingtin significantly. This clearly show, that P1 clears mutated Huntingtin only, the disease-causing protein in HD. Student's t-test was used for statistical analysis.

### Example 11: P1 Increases Active Mitochondrial Mass in a Cell Model of HD (ST HDH)

In addition to increasing both BDNF and lysosomal master regulator, TFEB, P1 (SEQ ID NO: 1) likewise increased mitochondrial master regulator PGC1a in both murine neurons and HD patient-derived fibroblasts. A consequence of PGC1a upregulation is the biogenesis of mitochondria and increased oxidative phosphorylation^{53,54}. Several mitochondrial deficits have been directly linked to Huntington's⁵⁵⁻⁶¹. Therefore, we assessed the role of P1 in regulating mitochondrial function as readout of PGC1a upregulation.

The impact on mitochondrial function was assessed in ST HDH cells (mouse striatal cell line) expressing either HTT with a 111 polyglutamine stretch (Q111) or a 7 polyglutamine stretch (Q7), thereby serving as a model of HD. The cells were stimulated with P1 at timepoints between 0-24 hours and mitochondrial mass was subsequently measured using MitoTracker—a probe which binds to active mitochondria. Signal was measured in plate-reader at ex/em 590/516.

As shown in FIG. 10, the sick cell line (Q111) displays lower base-levels of mitochondrial mass, than the healthy cell line (Q7). When stimulated with P1, the mitochondrial mass is increased in both Q7 and Q111 cells, while the mass in Q111 is increased above the baseline from the healthy cell line (at 24 hours). This demonstrates a therapeutic potential of P1 in targeting mitochondrial function in HD, and targeting mitochondrial dysfunction in other settings. Student's t-test was used for statistical analysis.

### Example 12: P1 Reaches Brain by Both Subcutaneous and Intravenous Injection

Both intravenous and subcutaneous delivery of drugs to the brain are considered challenging due to crossing of the blood-brain-barrier (BBB) among others. Therefore, many novel therapeutics for neurodegenerative diseases, such as antisense oligonucleotides, rely on intrathecal injections to circumvent any BBB-issues. As both subcutaneous and intravenous administrations are considered more patient-convenient and thus superior to intrathecal injections, we tested the ability of P1 (SEQ ID NO: 1) to reach the brain following intravenous and subcutaneous injections.

13 mg/kg and 52 mg/kg of P1 were either subcutaneously or intravenously injected in wild-type mice in either 4.38 mM L-His, 140 mM NaCl, 0.2% Tween-20 and 1500 IU hyaluronidase (for SC, pH 6.15) or saline (IV). Both plasma, whole brain and cerebrospinal fluid concentrations were validated at different timepoints of 0.25-4 hours by LC MS/MS.

FIG. 11A to FIG. 11C show brain and CSF levels of P1 following both SC and IV injections. T½ for P1 in brain following IV delivery were 0.232 hours, while SC delivery showed a T½ of 0.316 hours. The maximal brain-plasma ratio was 0.032 for SC delivery and 0.034 for IV delivery. As P1 displayed better half-life following SC injection, this route of administration was chosen in subsequent experiments.

Dose-Dependent Delivery

We further explored whether delivery to the CSF and brain would be dose-dependent by the SC route. P1 (SEQ ID NO: 1) were subcutaneously injected in wild-type mice in different concentrations of 0-52 mg/kg in 4.38 mM L-His, 140 mM NaCl, 0.2% Tween-20 and 1500 IU hyaluronidase (pH 6.15). Levels of P1 were validated in both plasma, whole brain and cerebrospinal fluid after 15 min injection by LC MS/MS.

P1 (FIG. 12A to FIG. 12C) was measurable in the brain and CSF at all concentrations. Levels in plasma, brain and CSF increased with the dosage, thereby displaying a dose-dependent delivery.

Formulation Composition

The composition of a formulation may impact stability, solubility and thus delivery of an active ingredient to target tissues, such as the brain. The initial L-his buffer used for SC administration contained the enzyme hyaluronidase as this is described to increase absorption of drugs injected subcutaneously. We therefore evaluated whether hyaluronidase is required for SC delivery to the brain.

13 mg/kg of P1 (SEQ ID NO: 1) were subcutaneously injected in wild-type mice in different formulations in either PBS buffer, in buffer containing 4.38 mM L-His, 140 mM NaCl, 0.2% Tween-20 and 1500 IU hyaluronidase (pH 6.15) or in buffer with 4.38 mM L-His, 140 mM NaCl, 0.2% Tween-20. Levels of P1 were validated in both plasma and whole brain after 15 and 30 min. of injection by LC MS/MS. P1 was measurable in the plasma and brain in all formulations while no significant difference was observed between L-His buffer with or without hyaluronidase. This suggests, that hyaluronidase is not required for brain delivery of P1 following SC injection (FIG. 13A to FIG. 13B).

### Example 13: Peptide P1 Displays High CREB-Activation in Striatum and Hippocampus of WT-Mice Following IV Injection

The aim of this study was to assess whether peptide P1 can cross the blood-brain-barrier (BBB) and activate CREB in the brain region of striatum in a wild-type mouse following intravenous injection. Loss of neurons within this region is the main hallmark in the neurodegenerative disease of Huntington's. Thus, assessing activation of CREB in this region would imply whether the peptide is able to penetrate the BBB to initiate pro-survival signals in brain regions affected in Huntington's disease.

To assess this, 8 weeks old wild-type mice were injected intravenously with either 0.26 mg/kg of peptide P1 or LP1 dissolved in saline. 1 hour after the injection, the mice were sacrificed by cervical dislocation and striatal tissue was isolated by dissection and immediately lysed using a TissueLyser in lysis buffer containing cOmplete and DTT. The samples were subsequently homogenized by sonication. The phosphorylation of CREB on serine 133 was subsequently validated by western blotting. The levels of phosphorylated CREB were normalized to the corresponding beta-actin levels. Student's t-test was used for statistical analysis.

As shown in FIG. 14, peptide P1 performed markedly better than LP1 in activating CREB, as demonstrated by its phosphorylation on serine 133, in both striatum (FIG. 14B) and hippocampus (FIG. 14A).

### Example 14: Subcutaneous Injection of Peptide P1 Activates Transcription Factors CREB and AMPK in Striatum of Wild-Type Mice

To assess the subcutaneous administration route of peptide P1, 8 weeks old wild-type mice were subcutaneously injected with different doses ranging between 0.13 to 26 mg/kg of peptide P1 dissolved in 4.38 mM L-His, 140 mM NaCl, 0.2% Tween-20 and 1500 IU hyaluronidase (pH 6.15). Likewise, was an earlier lead peptide constituting a linear version of P1 attached to a cell-penetrating moiety (P9, SEQ ID NO: 9), tested for its ability to activate CREB in both striatum and hippocampus (in a separate study) following subcutaneous injection with 3.6 mg/kg. 2 hours after injection the mice were sacrificed by cervical dislocation and striatal tissue was isolated by dissection and snap frozen in liquid nitrogen and stored at −80° C. until further use. To assess the activation of CREB and AMPK, the tissue was lysed using a TissueLyser in lysis buffer containing cOmplete and DTT. The samples were subsequently homogenized by sonication. The phosphorylation of CREB on serine 133 and threonine 172 on AMPK was subsequently validated by western blotting and the levels of were normalized to beta-actin. Student's t-test was used for statistical analysis.

The results demonstrate the ability of delivering peptide P1 to the striatum through subcutaneous administration—activating critical pathways in brain regions affected in Huntington's disease (FIG. 15A and FIG. 15B). Here peptide P9, was not able to notably activate CREB in either the striatum or hippocampus (FIG. 15C). Administration of 13 mg/kg resulted in the highest efficacy for P1.

In conclusion, peptide P1 can be delivered through subcutaneous injection to activate both AMPK and transcription factor CREB in the brain region of striatum in wild-type mice.

### Example 15: P1 Pathway Engagement In Vivo in Wild-Type Mice

As previously demonstrated, several proteins downstream of CREB, involved with lysosomal activation and mitochondrial biogenesis were regulated by P1 treatment (see Example 6). Similarly, we assessed the expression and regulation of these proteins in wild-type mice following SC injection to demonstrate target engagement in the striatum.

Wild-type mice were injected with 13 mg/kg of P1 (SEQ ID NO: 1) subcutaneously in 4.38 mM L-His, 140 mM NaCl, 0.2% Tween-20 and 1500 IU hyaluronidase (pH 6.15). The mice were sacrificed at timepoints between 2-8 hours after injection. Striatal tissue was isolated and the tissue was lysed using a TissueLyser in RIPA lysis buffer containing cOmplete and phosSTOP. Levels of pCREB, TFEB, downstream lysosomal gene products LAMP1, p62/SQSTM1, PGRN and mitochondrial master regulator PGC1a were validated by western blotting. All proteins were normalized to beta-actin levels.

As shown in FIG. 16A, P1 significantly activates CREB after 2 hours. Both TFEB and LAMP1 were significantly increased between 2-4 hours (FIG. 16B and FIG. 16C), while both PGRN & PGC1a were significantly increased at 6 hours (FIG. 16E and FIG. 16F). Furthermore, autophagic flux (a measured of autophagic degradation activity) was validated through assessing p62/SQSTM1 levels. P62 is a protein, which interacts with autophagic substrates and delivers them to autophagosomes for degradation⁴⁵. In the process, p62 is itself degraded and when autophagy is induced, a corresponding decrease in p62 levels should be observed. As shown in FIG. 16D, p62 levels declined after 4 hours and was significantly lowered at 8 hours following injection, demonstrating increased lysosomal activation in striatum of the wild-type mice. Student's t-test was used for statistical analysis.

### Example 16: Daily Subcutaneous Administration of P1 in R612 Mouse Model of HD

In a separate study, we validated target engagement of P1 (SEQ ID NO: 1) in R6/2 mice (mouse model of Huntington's), when treated for a prolonged period in the late stage of disease in this model. R6/2 mice were obtained from the Jackson Laboratory as:
FEMALE B6CBA-Tg(HDexon1)62Gpb/3J, stock 006494. Hemizygous (R6/2)
FEMALE B6CBA-Tg(HDexon1)62Gpb/3J, stock 006494. NonCarrier (WT controls)

Mice were delivered at 5 weeks of age and maintained on standard diet and water adlib, and a 7 am to 7 μm day-cycle.

The R6/2 mice were injected with a daily dose of 13 mg/kg of P1 subcutaneously in 4.38 mM L-His, 140 mM NaCl, 0.2% Tween-20 and 1500 IU hyaluronidase (pH 6.15) between 8 weeks to 12 weeks of age. At 12 weeks of age, mice were sacrificed and their brains removed and both hippocampus—cortex and striatal tissue was isolated and lysed in RIPA lysis buffer containing containing cOmplete and phosSTOP.

Measurement of Neurotrophic and Mitochondrial Proteins

Levels of both BDNF and its receptor, TrkB, were validated along with levels of the mitochondrial master regulator PGC1a. In addition, levels of DARPP32, a marker of medium spiny neurons (MSN), which are the neurons that ultimately perish in HD, were evaluated in cortex tissue.

As shown in FIG. 17, mice treated with P1 demonstrated significantly higher basal levels of both BDNF (FIG. 17B), TrkB (FIG. 17C) and PGC1a (FIG. 17D). In addition, a tendency to increase the levels of DARPP32 was observed (FIG. 17A).

Thus, subcutaneous administration of P1 (SEQ ID NO: 1) targets key pathways and upregulate vital proteins in HD important to neurotrophic support and synaptic- and mitochondrial function.

Brain Weight and Activity Studies

Prior to treatment (8 weeks of age), the R6/2 mice were weighed and assessed for clasping and rotarod behavior before randomization to 4 cages of n=6 each (two cages pr treatment group). The experimenter remained blinded throughout the studies and data analysis. Motor coordination was measured by hind-limb clasping and rotarod between week 7-12.

Clasping: Mice were once weekly assessed for clasping behavior. Mice were placed on the top grid of a cage and then held by the tail and lifted over the top of the homecage for 10 successive trials of 3 seconds with 3 second rest intervals

Each sequence of 3 seconds was scored from 0-3:

0: Normal righting response/no certain abnormalities. Slight collapse of the hind limbs toward the midline is not scored as abnormal unless the hind limbs are at least parallel. Struggling to grab hind limbs or tail with forelimbs are not scored as abnormal.

1: 1 hind limb with abnormal retraction or clearly abnormal posture, or both hind limbs abnormally collapsing toward midline until they are at least parallel but without touching or crossing.

2: Both hind limbs clasping (touching or crossing) after a delay of more than one second or intermittently.

3: Almost immediate (within the first second) and persistent clasping of hind limbs.

Rotarod: Accelerating test—cylinder accelerates from 4 to 40 RPM in 5 minutes with linear speed progression. During the first week of training, mice undergo 3 trials per day for 3 consecutive days. During training sessions, mice receive “second changes” when falling from the rotarod. The following weeks, mice are tested on only one day for 3 consecutive trials. The mean latency to fall is used for data analysis.

At 11 weeks of age, mice were subjected to an open field consisting of a 40×40 cm box with transparent Plexiglas walls of 50 cm height. Their behaviour was recorded using an automated tracking software and camera (Anymaze, Havard apparatus). Distance travelled and number of rears were recorded over a 20 min. period.

Brain weight: At 12 weeks of age, mice were sacrificed and the entire brain was carefully extracted and weighed using a fine-scale weigh.

Despite starting administration of P1 at very late stages of disease development in the R6/2 model, P1-treatment had a tendency to increase both brain weight (FIG. 18A), distance travelled by mice (FIG. 18B), lower hind-limb clasping (FIG. 18D) and increase performance on rotarod (FIG. 18E). In addition, the rearing frequency (a measure of activity behaviour) was increase in treated mice compared to vehicle treated controls (FIG. 18C).

Stereological Analysis

The brain was cut using a fine razor blade and one hemisphere (right) was directly put into ice-cold 4% PFA in PBS for 24 hours, and then transferred to a 30% sucrose PBS solution for 48 hours and stored at 4° C.

The PFA fixed hemispheres were imbedded in TissueTec and sliced on a cryostat at 50 μm thickness. For stereology, every 5th section was sampled using the principle of systematic random sampling, giving a section sampling fraction (SSF)=1/5.

The sections were mounted on chrome-gelatin-coated slides and Nissl stained with a 0.25% thionine solution (thionin, Sigma T3387). Image acquisition and analysis was performed using the newCAST system (Visiopharm, Horsholm, Denmark). This system consists of an Olympus light microscope (Olympus BX50, Olympus, Denmark) modified for stereology with a digital camera (PixeLINK PLA686C, Canada) and a motorized microscope stage (Prior H138 with controller H29, Cambridge, UK). The newCAST software was interfaced to the digital camera superimposing the counting frames on the live images.

The volume estimation was carried out using the Cavalieri principle for quantifying the total volume of the hippocampus, striatum and mid-part of the cortex (6 slides used for this) using a 4× lens.

While initiating treatment at a late stage of disease progression in this mouse model, P1 treatment had a tendency of increasing both hippocampal and cortical volume (FIG. 19A and FIG. 19B). Student's t-test was used for statistical analysis.

### Example 17: P1 Stability Assays in Plasma and Brain Homogenates

An important aspect of drug-development is pharmacokinetics, including absorption, distribution, metabolism and excretion. We next evaluated the stability of P1 (SEQ ID NO: 1) in both human and mouse plasma and mouse brain homogenate samples as well as plasma and brain binding. High plasma binding may act as a reservoir or depot, which is slowly released as the unbound form to target tissues. As unbound forms are being metabolized and/or excreted from the body, a high plasma binding affects half-life of the drug.

Plasma Stability

Mouse or human plasma were incubated with 2 μM of P1 (SEQ ID NO: 1) or propantheline bromide (positive control for degradation) and left at 37° C. At each time point, stop solution was added to precipitate the solution and after mixing and centrifugation, supernatant was used for LCMS analysis.

For plasma binding, 2 μM P1 or Warfarin (positive control) were added to mouse or human plasma. After 30 minutes incubation at 37° C., samples where ultracentrifuged for 4 hours to generate protein-free samples for LCMS analysis. In parallel, plasma was incubated for 4.5 h to determine total levels after incubation. Plasma binding was calculated as % Bound=100*(1−F/T4.5), where F is concentration of free ligand and T4.5 is the concentration of total ligand after incubation.

As demonstrated by FIG. 20A and FIG. 20C, P1 had a half-life of more than 289 minutes and more than 75% of the compound being unbound in plasma of both mouse and human (FIG. 20B and FIG. 20D).

Mouse Brain Stability

Mouse brain homogenate was incubated with 2 μM of P1 or 7-Ethoxycoumarin (positive control of degradation). Enzymatic reactions were stopped at timepoints between 0-240 min and samples analysed by LCMS. For test with protease inhibitors, brain homogenate was pre-incubated for 30 minutes with protease inhibitors prior to incubation with P1. For brain homogenate binding, 2 μM of P1 or propranolol (positive control) were added to mouse brain homogenate and incubated at 37° C. for 30 minutes. To prepare the protein-free samples (F samples) to be used for unbound determination, an aliquot of the pre-incubated matrix containing test compound or control compound was transferred to ultracentrifuge tubes and subjected to ultracentrifugation at 37° C., 35000 rpm for 4 hrs. In parallel, brain homogenate was incubated for 4.5 hrs to determine total levels after incubation.

As demonstrated by FIG. 20, P1 shows T½ of 245 minutes in brain homogenate (FIG. 20E) and less than 20% binding (FIG. 20F).

### Example 18: P1 Metabolic Stability in Liver Fractions

As the majority of drug metabolism occurs in the liver, liver in vitro preparations may serve as models to evaluate metabolic stability of drugs. Frequently used in vitro models are 1) S9 fractions (containing cytosol and microsomes with enzymatic activities) and 2) liver microsomes only.

Liver S9 Fraction Stability

S9 fractions from 5 different species were incubated with 2 μM P1 (SEQ ID NO: 1) or 7-Ethoxycoumarin (positive control for clearance) and necessary reactants for up to 60 minutes at 37° C. Reactions were stopped and samples were analysed by LCMS. As shown in FIG. 21A, P1 has low clearance and high stability in liver S9 fractions, although some stability is lost in mouse and human S9 liver fractions.

Liver Microsome Stability

Liver microsomes from mouse and human were incubated with 2 μM of P1 or diclofenac (positive control for degradation) and necessary reactants for up to 60 minutes at 37° C. Reactions were stopped at timepoints between 0-60 min. and samples analysed by LCMS. As demonstrated by FIG. 21C and FIG. 21D, P1 shows low clearance and high stability in both human and mouse liver microsomes.

### Example 19: P1 Stability in Buffer Formulation

Buffer formulations may impact the stability of drugs at different storage conditions. We assessed whether the L-His buffer composition (with hyaluronidase) would impact the stability of P1 (SEQ ID NO 1).

Buffer solution containing 4.4 mM L-histidine, 140 mM NaCl, 0.2% w/V Tween 20 and 1500 IU/mL hyaluronidase was incubated with 1 mM P1 at −20° C., 4° C. and 25° C. for up to 7 days. LCMS was used to ascertain the amount of P1 remaining in the solutions. FIG. 22 show no stability issues at −20° C. and 4° C., while 25° C. could affect stability after 4 days.

### Example 20: Cytochrome P450 Inhibition

The liver is highly susceptible to drug-induced toxicity as drugs are concentrated there. Therefore, liver toxicity is also a leading cause for removal of existing marketed drugs or hindrance in drug development. Thus, we assessed the potential toxic effects of P1 on liver enzymes P450, a class of enzymes important for drug clearance.

Liver microsomes were prepared with cocktails of known substrates of the respective CYP450 enzymes to evaluate the effects of P1 on enzyme inhibition. For each CYP-enzyme a previously described inhibitor was used as a positive control. Sample solutions were pre-incubated with P1 (10 mM) or inhibitor at 37° C. for ten minutes before addition of NADPH. Enzyme activity was measured in samples containing a dose-range of P1 with or without NAPDH (substrate used by the liver enzymes in detoxification steps). Reactions were incubated for 10 minutes before the reactions were stopped and analysed by LCMS to detect CYP metabolites.

FIG. 23A to FIG. 23G show that P1 (SEQ ID NO: 1) has no CYP-inhibition effects neither with nor without NADPH, indicating no toxicologic effect of P1.

### Example 21: hERG Inhibition

The cardiac hERG potassium channel is responsible for a rapid delayed rectifier current (IKr) in human ventricles. Inhibition of IKr is the most common mechanism of the non-cardiac drug evoked ventricular action potential duration increase. The increased action potential duration causes prolongation of the QT interval in the electrocardiogram that is associated with a dangerous ventricular arrhythmia, named torsade de pointes. Therefore, testing the interaction of a compound with the hERG potassium channel in heterologous expression systems is recommended by the International Conference on Harmonisation (ICH) as one of the non-clinical testing methods for assessing the potential of a test compound to prolong the QT interval.

P1 (0.1-30 uM) was evaluated in vitro in a concentration-response (relationship of the effect on electric current passing through hERG (human ether-à-go-go-related gene) potassium channels (a surrogate for IKr, the rapidly activating, delayed rectifier cardiac potassium current) stably expressed in a CHO cell line using a manual patch-clamp technique.

The hERG current was recorded at room temperature using whole-cell patch-clamp techniques. Output signals from the patch-clamp amplifier were digitized and low-pass filtered at 2.9 kHz. The recording was controlled with Patchmaster Pro software. The recording chamber with cells seeded was mounted on an inverted microscope stage. A cell in the recording chamber was randomly picked up for testing. The cell was continuously perfused from the perfusion system. A micropipette filled with ICS was used as recording electrode in the manual patch-clamp study. The micropipette was prepared on the day of the patchclamp experiment using glass capillaries (GC150TF-10, Harvard Apparatus Co., UK). From the holding potential of −80 mV, the voltage was increased to +60 mV for 850 ms to open the hERG channels. After that, the voltage was decreased to −50 mV for 1275 ms, causing a “rebound” or tail current, the peak tail current was measured and collected for data analysis. Finally, the voltage was decreased to the holding potential (−80 mV). This command voltage protocol was repeated every 15 s continuously during P1 application. Cells were then perfused with P1 or positive control working solutions until the peak tail current amplitude reach a stable state. At this point, cells will be once again perfused with next concentration of P1.

FIG. 24 shows that P1 had no effect on hERG current at the tested concentrations between 0.1-30 uM.

### Example 22: Peptide P1 Increases Clearance of Cytoplasmic TDP-43

The sortilin family has been associated with both frontotemporal dementia (FTD) and ALS^{20,23}—a disease in which the transcriptional repressor protein TARDBP (TDP-43) aggregates in the cytoplasm^{65,66}. As P1 both increases lysosomal acidification and biogenesis to induce autophagy, in this study the ability of peptide P1 to increase the clearance of cytoplasmic TDP-43 in HEK293 cells was assessed, an approach for treating FTD.

100,000 HEK293T cells were seeded in a 24-well plate per well. At 60% confluency the cells were transfected with a form of TDP-43-YFP with a mutated nuclear localization signal (TDP-43(ΔNLS)-YFP) to restrict TDP-43 to the cytosol, which is the pathogenic localization in frontotemporal dementia⁶⁷. After 24 hours, the cells were treated with a dose-range of peptide P1 and further incubated for another 24 hours. The cells were subsequently lysed in lysis buffer containing DTT and cOmplete cocktail protease inhibitor. The levels of TDP-43 were validated by western blotting using an anti-GFP antibody and the levels were normalized to beta-actin.

The results, shown in FIG. 25, demonstrate that peptide P1 decreased the pathogenic cytoplasmic form of TDP-43, indicating a therapeutic potential in frontotemporal dementia and ALS.

### Example 23: Peptide P1 Increases Branching of Progranulin (GRN)-Deficient Neurons

To further validate the therapeutic potential of peptide P1 in frontotemporal dementia, its neurotrophic activity in GRN heterozygous hippocampal neurons isolated from p0 mice was investigated. Heterozygous loss of function mutations in the progranulin gene, GRN, cause frontotemporal dementia⁶⁸. To assess this, we measured the dendritic branching of GRN+/− hippocampal neurons in vitro following treatment. 10000 neurons were seeded per coverslip. After 24 hours the medium was changed to medium containing 1 uM of peptide P1. Hereafter, the cells were incubated at 37° C. for 72 hours before being fixed in 4% PFA for 20 minutes at room temperature. The neurons were subsequently stained against MAP2 to determine cell morphology. Images were taken using a confocal microscope and the neurite branches were analysed using Zen Image Processing (Carl Zeiss). The total number of branches were calculated per neuron. Student's t-test was used for statistical analysis.

As shown in FIG. 26, treating neurons suffering from frontotemporal dementia resulted in increased branching of the neurons, which displays the neurotrophic activity of peptide P1 in this neurodegenerative disease.

### Example 24: Peptide P1 Activates Transcription Factor CREB in Progranulin (GRN)-Deficient Neurons

In this study, it was examined whether peptide P1 could increase phosphorylation of CREB (ser133) similar to wild-type neurons. To study the activation of CREB, cortical neurons were isolated from GRN+/−p0 wild-type mice and seeded in a density of 500,000 per well. After 7 days in vitro the neurons were stimulated with peptide P1 at 1 uM in neurobasal A media at 37° C. and 5% CO₂ and incubated for 20 minutes. Following, the neurons were lysed in lysis buffer containing DTT and cOmplete cocktail protease inhibitor and subsequently sonicated to disrupt the nuclear membrane. The phosphorylation of CREB on serine 133 was subsequently validated by western blotting. The levels of phosphorylated CREB were normalized to the corresponding beta-actin levels. Student's t-test was used for statistical analysis.

As shown in FIG. 27, peptide P1 was able to activate CREB in an in vitro model of frontotemporal dementia (GRN+/−).

### Example 25: Peptides P2, P4 and P6 Increase Survival in GRN-Deficient Cortical Neurons

One consequence of CREB-activation is the upregulation of pro-survival genes leading to decreased apoptotic signalling and increased neuroprotection. It was thus investigated if treating GRN(+/−) cortical neurons (a model of frontotemporal dementia) would increase their ability to survive. This was assessed in primary neuronal cultures, as these spontaneously disintegrate and die as they get older in vitro.

Cortical neurons were isolated from p0 wild-type mice and seeded in a density of 50,000 in a 96-well plate pre-coated with poly-L and laminin and incubated at 37° C. in a 5% C02 atmosphere. At 7 days in vitro (DIV) the neurons were treated by changing half the media with neurobasal A with B27 containing the peptides (P2, P4 or P6) to give a finale concentration of 1 uM and further incubated. The cortical neurons were treated in a likewise manner on DIV9, DIV11, DIV13 and DIV15. At DIV16 half the media was removed and 3-(4,5-dimethylthiazole-2-yl)-2,5-diphenyltetrazolium bromide (MTT) was added to the media to give a finale concentration of 0.5 mg/mL. MTT is reduced by mitochondrial enzymes in active cells to an insoluble product called formazan, which can be detected by colorimetry. Thus, the colour generated reflects activity and viable cells in the culture. The neurons were incubated for 4 hours with MTT and thereafter lysed in a 50% EtOH and 50% DMSO solution. To assure proper mixing of formazan, the plate was left to shake for 30 minutes before being measured at 570 nm and 650 nm. Student's t-test was used for statistical analysis.

The results are depicted in FIG. 28A, FIG. 28B and FIG. 28C as the relative survival compared to control (neurons treated with a scrambled peptide). Peptides P2, P4 and P6 increased the relative survival compared to non-treated.

In conclusion, treatment with peptides P2, P4 and P6 caused an increase in survival of GRN-deficient cortical neurons.

### Example 26: Peptide P1 Increases Lysosomal Acidification

Primary hippocampal neurons from GRN+/− mice were seeded in an 8-well ibidi chamber pre-coated with Poly-I and laminin. 75,000 neurons were seeded pr. well. After 5 days in vitro the neurons were stimulated for 4, 8 or 24 hours with peptide P1 at 1 uM in neurobasal A with B27. After stimulation, the medium was changed to pre-warmed (37° C.) neurobasal A containing 1 uM of LysoSensor probe DND-189 (an acidotropic probes that accumulates in acidic organelles as the result of protonation) and incubated for 30 min. at 37° C. in a 5% C02 atmosphere. Hereafter, the media was changed to pre-warmed FluoroBrite Medium and the neurons were imaged in Olympus microscopy system. Pictures were taken of the lysosomes and processed using the ScanR imaging software to give fluorescence (intensity) of each lysosomal vesicle. The summed total intensity of all lysosomes was calculated and divided by the total number of neurons imaged yielding the total intensity of lysosomes per neuron. Student's t-test was used for statistical analysis.

The results are shown in FIG. 29. After 4 hours of treatment, the lysosomal intensity was increased in P1 treated GRN+/− neurons, demonstrating an increase in the acidification of lysosomes. This demonstrates that peptide P1 increases lysosomal activity in an in vitro model of FTD.

### Example 27: Peptide P1 Increases Granulin Levels in Wild-Type Mice Following 7 Days Daily Treatment

Increasing levels of GRN are considered a therapeutic approach in FTD. Interestingly, GRN contain a transcription factor binding site at its promotor site for TFEB, suggesting TFEB is regulating the expression of GRNs⁶⁹. As we previously showed to increase TFEB, we assessed whether P1-treatment could increase GRN-levels in WT mice.

Wild-type mice were injected with a daily dose of 13 mg/kg of P1 (SEQ ID NO: 1) subcutaneously in 4.38 mM L-His, 140 mM NaCl, 0.2% Tween-20 and 1500 IU hyaluronidase (pH 6.15) for 7 days. The mice were sacrificed on day 8. The hippocampus was isolated and lysed using a TissueLyser in RIPA lysis buffer containing cOmplete and phosSTOP. Levels of GRN (ab HPA008763 from Sigma) was validated by western blotting normalized to beta-actin. Student's t-test was used for statistical analysis.

As shown in FIG. 30, mice treated with P1 demonstrated higher levels of GRN. This demonstrates the therapeutic value of P1 in FTD patients carrying heterozygous GRN mutations.

### Example 28: Peptide P3 Acutely Attenuates Neuropathic Pain in a Spared Nerve Injury Mouse Model

To assess the effect of peptide P3 on neuropathic pain, the spared nerve injury (SNI) model was used. The threshold for mechanical pain response is determined by testing with von Frey filaments of increasing bending force, which are repetitively pressed against the lateral area of the paw. Two baseline measurements were made before SNI operation. In brief, the common peroneal and tibial branches of the sciatic nerve were ligated and cut distally to the ligation, just distal to the branching of the sural nerve, which was left untouched. The mechanical allodynia was subsequently assessed with von Frey testing 17 days post-surgery. As depicted in FIG. 31, a single dose of peptide P3 by subcutaneous administration 17 days post-SNI operation in 4.38 mM L-His, 140 mM NaCl and 0.2% maltoside attenuated the pain in an acute manner measured by von Frey test, for up to 2½ hours. Student's t-test was used for statistical analysis.

In conclusion, peptide P3 acutely attenuates neuropathic pain caused by the SNI model for up to 2½ hours, indicating a therapeutic potential of peptide P3 in treating individuals suffering from injury-related neuropathic pain.

### Example 29: Peptide P3 Reduces Neuropathic Pain in a Spared Nerve Injury Mouse Model

Mice, surgery and von Frey testing was performed as in Example 28.

As depicted in FIG. 32, a single dose daily for 8 days of peptide P3 by subcutaneous administration 1 day post-SNI operation in 4.38 mM L-His, 140 m M NaCl and 0.2% maltoside in a SNI mouse model, continuously reduced the neuropathic pain measured by von Frey test as demonstrated by attenuated pain before the acute injection on day 8. Student's t-test was used for statistical analysis.

In conclusion, daily delivery of peptide P3 attenuates neuropathic pain caused by the SNI model, indicating a therapeutic potential of peptide P3 in treating individuals suffering from injury-related neuropathic pain.

### Example 30: Peptide P6 Increases Neuronal Branching

SorCS3 has been genome-widely implicated in multiple neurodevelopment-related traits and found to be associated with ADHD, depression, schizophrenia, autism and bipolar disorder as well as Alzheimer's^{70,71}. As growth by neurite elongation and branching are important in the development of neurons, we assessed whether P6 (SEQ ID NO: 6), derived from SorCS3-receptor, could increase neuronal branching.

Hippocampal neurons were isolated from p0 wild-type mice and seeded in a density of 10.000 per well in 24-well trays containing poly-D and laminin coated coverslips. At DIV1 the neurons were treated with 0.1 uM or 1 uM P6. BDNF (1 nM) was used as a positive control. At DIV4 the neurons were fixed and immunostained for MAP2, a marker of dendrites. Pictures of MAP2 were taken of neurites with 20 neurons pr. coverslip. The neurites may not be in contact with other neurites from other neurons. The pictures were analysed in Imaris Software. The number branches were counted manually.

As shown in FIG. 33, P6 increased neurite branching similar to BDNF at both 0.1 uM and 1 uM concentration, which demonstrates neurotrophic activity of P6. Student's t-test was used for statistical analysis.

### Example 31: Peptide P6 Increases Synaptic Vesicle Glycoprotein 2A (SV2A)

SV2A levels are strongly positively correlated with synaptophysin levels in the brain, which is reduced in disorders associated with synaptic loss, and thus used as a marker of synaptic density⁷². Several associated diseases with SorCS3 show synaptic loss, including Alzheimer's and schizophrenia. We there studied if the SorCS3-derived peptide, P6 (SEQ ID NO: 6) could affect SV2A levels in vitro.

Hippocampal neurons were isolated from p0 wild-type mice and seeded in a density of 250,000 per well in 12-well trays precoated with poly-L and laminin. At DIV8 the neurons were treated with 0.1 uM or 1 uM P6 for 3 days. The neurons were lysed in RIPA buffer containing cOmplete and phosSTOP and SV2A levels were validated by western blotting, normalized to beta-actin.

FIG. 34 shows that P6-treated neurons had increased SV2A levels, suggesting a therapeutic potential in neurodegenerative or psychiatric diseases with synaptic loss. Student's t-test was used for statistical analysis.

### REFERENCES

1 Benito, E. & Barco, A. CREB's control of intrinsic and synaptic plasticity: implications for CREB-dependent memory models. Trends Neurosci 33, 230-240, doi:10.1016/j.tins.2010.02.001 (2010).
2 Sakamoto, K., Karelina, K. & Obrietan, K. CREB: a multifaceted regulator of neuronal plasticity and protection. J Neurochem 116, 1-9, doi:10.1111/j.1471-4159.2010.07080.x (2011).
3 Sugars, K. L., Brown, R., Cook, L. J., Swartz, J. & Rubinsztein, D. C. Decreased cAMP response element-mediated transcription: an early event in exon 1 and full-length cell models of Huntington's disease that contributes to polyglutamine pathogenesis. J Biol Chem 279, 4988-4999, doi:10.1074/jbc.M310226200 (2004).
4 Pugazhenthi, S., Wang, M., Pham, S., Sze, C. I. & Eckman, C. B. Downregulation of CREB expression in Alzheimer's brain and in Abeta-treated rat hippocampal neurons. Mol Neurodegener 6, 60, doi:10.1186/1750-1326-6-60 (2011).
5 Ljungberg, M. C. et al. CREB-activity and nmnat2 transcription are down-regulated prior to neurodegeneration, while NMNAT2 over-expression is neuroprotective, in a mouse model of human tauopathy. Hum Mol Genet 21, 251-267, doi:10.1093/hmg/ddr492 (2012).
6 Boland, B. et al. Promoting the clearance of neurotoxic proteins in neurodegenerative disorders of ageing. Nat Rev Drug Discov 17, 660-688, doi:10.1038/nrd.2018.109 (2018).
7 Qin, Z. H. et al. Huntingtin bodies sequester vesicle-associated proteins by a polyproline-dependent interaction. J Neurosci 24, 269-281, doi:10.1523/JNEUROSCI.1409-03.2004 (2004).
8 Wong, Y. C. & Holzbaur, E. L. The regulation of autophagosome dynamics by huntingtin and HAP1 is disrupted by expression of mutant huntingtin, leading to defective cargo degradation. J Neurosci 34, 1293-1305, doi:10.1523/JNEUROSCI.1870-13.2014 (2014).
9 van Swieten, J. C. & Heutink, P. Mutations in progranulin (GRN) within the spectrum of clinical and pathological phenotypes of frontotemporal dementia. Lancet Neurol 7, 965-974, doi:10.1016/S1474-4422(08)70194-7 (2008).
10 Beel, S. et al. Progranulin reduces insoluble TDP-43 levels, slows down axonal degeneration and prolongs survival in mutant TDP-43 mice. Mol Neurodegener 13, 55, doi:10.1186/s13024-018-0288-y (2018).
11 Savas, J. N. et al. The Sorting Receptor SorCS1 Regulates Trafficking of Neurexin and AMPA Receptors. Neuron 87, 764-780, doi:10.1016/j.neuron.2015.08.007 (2015).
12 Subkhangulova, A. et al. SORCS1 and SORCS3 control energy balance and orexigenic peptide production. EMBO Rep 19, doi:10.15252/embr.201744810 (2018).
13 Breiderhoff, T. et al. Sortilin-related receptor SORCS3 is a postsynaptic modulator of synaptic depression and fear extinction. PLoS One 8, e75006, doi:10.1371/journal.pone.0075006 (2013).
14 Hermey, G. et al. SorCS1 variants and amyloid precursor protein (APP) are co-transported in neurons but only SorCS1c modulates anterograde APP transport. J Neurochem 135, 60-75, doi:10.1111/jnc.13221 (2015).
15 Glerup, S. et al. SorCS2 is required for BDNF-dependent plasticity in the hippocampus. Mol Psychiatry 21, 1740-1751, doi:10.1038/mp. 2016.108 (2016).
16 Leloup, N., Chataigner, L. M. P. & Janssen, B. J. C. Structural insights into SorCS2-Nerve Growth Factor complex formation. Nat Commun 9, 2979, doi:10.1038/s41467-018-05405-z (2018).
17 Christiansen, G. B. et al. The sorting receptor SorCS3 is a stronger regulator of glutamate receptor functions compared to GABAergic mechanisms in the hippocampus. Hippocampus 27, 235-248, doi:10.1002/hipo.22689 (2017).
18 Baum, A. E. et al. A genome-wide association study implicates diacylglycerol kinase eta (DGKH) and several other genes in the etiology of bipolar disorder. Mol Psychiatry 13, 197-207, doi:10.1038/sj.mp.4002012 (2008).
19 Ollila, H. M. et al. Findings from bipolar disorder genome-wide association studies replicate in a Finnish bipolar family-cohort. Mol Psychiatry 14, 351-353, doi:10.1038/mp. 2008.122 (2009).
20 Hu, F. et al. Sortilin-mediated endocytosis determines levels of the frontotemporal dementia protein, progranulin. Neuron 68, 654-667, doi:10.1016/j.neuron.2010.09.034 (2010).
21 He, Y., Fang, Z. & Yu, G. Sortilin-related VPS10 domain containing receptor 1 and Alzheimer's disease-associated allelic variations preferentially exist in female or type 2 diabetes mellitus patients in southern Han Chinese. Psychogeriatrics 12, 215-225, doi:10.1111/j.1479-8301.2012.00405.x (2012).
22 Alemany, S. et al. New suggestive genetic loci and biological pathways for attention function in adult attention-deficit/hyperactivity disorder. Am J Med Genet B Neuropsychiatr Genet 168, 459-470, doi:10.1002/ajmg.b.32341 (2015).
23 Mori, F. et al. Sortilin-related receptor CNS expressed 2 (SorCS2) is localized to Bunina bodies in amyotrophic lateral sclerosis. Neurosci Lett 608, 6-11, doi:10.1016/j.neulet.2015.09.030 (2015).
24 Ma, Q. et al. SorCS2-mediated NR2A trafficking regulates motor deficits in Huntington's disease. JCI Insight 2, doi:10.1172/jci.insight.88995 (2017).
25 Hermey, G. et al. Characterization of sorCS1, an alternatively spliced receptor with completely different cytoplasmic domains that mediate different trafficking in cells. J Biol Chem 278, 7390-7396, doi:10.1074/jbc.M210851200 (2003).
26 Oetjen, S., Mahlke, C., Hermans-Borgmeyer, I. & Hermey, G. Spatiotemporal expression analysis of the growth factor receptor SorCS3. J Comp Neurol 522, 3386-3402, doi:10.1002/cne.23606 (2014).
27 Malik, A. R. et al. SorCS2 facilitates release of endostatin from astrocytes and controls post-stroke angiogenesis. Glia, doi:10.1002/glia.23778 (2020).
28 Malik, A. R. et al. SorCS2 Controls Functional Expression of Amino Acid Transporter EAAT3 and Protects Neurons from Oxidative Stress and Epilepsy-Induced Pathology. Cell Rep 26, 2792-2804 e2796, doi:10.1016/j.celrep.2019.02.027 (2019).
29 Goodarzi, M. O. et al. SORCS1: a novel human type 2 diabetes susceptibility gene suggested by the mouse. Diabetes 56, 1922-1929, doi:10.2337/db06-1677 (2007).
30 Lane, R. F. et al. Diabetes-associated SorCS1 regulates Alzheimer's amyloid-beta metabolism: evidence for involvement of SorL1 and the retromer complex. J Neurosci 30, 13110-13115, doi:10.1523/jneurosci.3872-10.2010 (2010).
31 Paterson, A. D. et al. A genome-wide association study identifies a novel major locus for glycemic control in type 1 diabetes, as measured by both A1C and glucose. Diabetes 59, 539-549, doi:10.2337/db09-0653 (2010).
32 Schmidt, V. & Willnow, T. E. Protein sorting gone wrong—VPS10P domain receptors in cardiovascular and metabolic diseases. Atherosclerosis 245, 194-199, doi:10.1016/j.atherosclerosis.2015.11.027 (2016).
33 Wang, H. F. et al. SORCS1 and APOE polymorphisms interact to confer risk for late-onset Alzheimer's disease in a Northern Han Chinese population. Brain research 1448, 111-116, doi:10.1016/j.brainres.2012.01.067 (2012).
34 Binzer, S. et al. Genetic analysis of the isolated Faroe Islands reveals SORCS3 as a potential multiple sclerosis risk gene. Multiple sclerosis (Houndmills, Basingstoke, England) 22, 733-740, doi:10.1177/1352458515602338 (2016).
35 Cross-Disorder Group of the Psychiatric Genomics Consortium. Electronic address, p. m. h. e. & Cross-Disorder Group of the Psychiatric Genomics, C. Genomic Relationships, Novel Loci, and Pleiotropic Mechanisms across Eight Psychiatric Disorders. Cell 179, 1469-1482 e1411, doi:10.1016/j.cell.2019.11.020 (2019).
36 Demontis, D. et al. Discovery of the first genome-wide significant risk loci for attention deficit/hyperactivity disorder. Nature genetics, doi:10.1038/s41588-018-0269-7 (2018).
37 Wray, N. R. et al. Genome-wide association analyses identify 44 risk variants and refine the genetic architecture of major depression. Nature genetics 50, 668-681, doi:10.1038/s41588-018-0090-3 (2018).
38 Walton, M. R. & Dragunow, I. Is CREB a key to neuronal survival? Trends Neurosci 23, 48-53, doi:10.1016/s0166-2236(99)01500-3 (2000).
39 Tao, X., Finkbeiner, S., Arnold, D. B., Shaywitz, A. J. & Greenberg, M. E. Ca2+ influx regulates BDNF transcription by a CREB family transcription factor-dependent mechanism. Neuron 20, 709-726, doi:10.1016/s0896-6273(00)81010-7 (1998).
40 Kang, H. et al. Activation of the ATF2/CREB-PGC-1alpha pathway by metformin leads to dopaminergic neuroprotection. Oncotarget 8, 48603-48618, doi:10.18632/oncotarget.18122 (2017).
41 Seok, S. et al. Transcriptional regulation of autophagy by an FXR-CREB axis. Nature 516, 108-111, doi:10.1038/nature13949 (2014).
42 Xing, F. et al. The Anti-Warburg Effect Elicited by the cAMP-PGC1alpha Pathway Drives Differentiation of Glioblastoma Cells into Astrocytes. Cell Rep 18, 468-481, doi:10.1016/j.celrep. 2016.12.037 (2017).
43 Binder, J. L., Chander, P., Deretic, V., Weick, J. P. & Bhaskar, K. Optical induction of autophagy via Transcription factor EB (TFEB) reduces pathological tau in neurons. PLoS One 15, e0230026, doi:10.1371/journal.pone.0230026 (2020).
44 Zhuang, X. X. et al. Pharmacological enhancement of TFEB-mediated autophagy alleviated neuronal death in oxidative stress-induced Parkinson's disease models. Cell Death Dis 11, 128, doi:10.1038/s41419-020-2322-6 (2020).
45 Bjorkoy, G. et al. Monitoring autophagic degradation of p62/SQSTM1. Methods Enzymol 452, 181-197, doi:10.1016/S0076-6879(08)03612-4 (2009).
46 Paquette, M. et al. AMPK-dependent phosphorylation is required for transcriptional activation of TFEB and TFE3. Autophagy, 1-19, doi:10.1080/15548627.2021.1898748 (2021).
47 Willows, R. et al. Phosphorylation of AMPK by upstream kinases is required for activity in mammalian cells. Biochem J 474, 3059-3073, doi:10.1042/BCJ20170458 (2017).
48 Gwinn, D. M. et al. AMPK phosphorylation of raptor mediates a metabolic checkpoint. Mol Cell 30, 214-226, doi:10.1016/j.molcel.2008.03.003 (2008).
49 Bok, J., Wang, Q., Huang, J. & Green, S. H. CaMKII and CaMKIV mediate distinct prosurvival signaling pathways in response to depolarization in neurons. Mol Cell Neurosci 36, 13-26, doi:10.1016/j.mcn.2007.05.008 (2007).
50 Racioppi, L. & Means, A. R. Calcium/calmodulin-dependent protein kinase kinase 2: roles in signaling and pathophysiology. J Biol Chem 287, 31658-31665, doi:10.1074/jbc.R112.356485 (2012).
51 Sabbir, M. G. CAMKK2-CAMK4 signaling regulates transferrin trafficking, turnover, and iron homeostasis. Cell Commun Signal 18, 80, doi:10.1186/s12964-020-00575-0 (2020).
52 Vilchez, D., Saez, I. & Dillin, A. The role of protein clearance mechanisms in organismal ageing and age-related diseases. Nat Commun 5, 5659, doi:10.1038/ncomms6659 (2014).
53 Cheng, A. et al. Involvement of PGC-1alpha in the formation and maintenance of neuronal dendritic spines. Nat Commun 3, 1250, doi:10.1038/ncomms2238 (2012).
54 Wareski, P. et al. PGC-1{alpha} and PGC-1{beta} regulate mitochondrial density in neurons. J Biol Chem 284, 21379-21385, doi:10.1074/jbc.M109.018911 (2009).
55 Marullo, M. et al. Analysis of the repressor element-1 silencing transcription factor/neuron-restrictive silencer factor occupancy of non-neuronal genes in peripheral lymphocytes from patients with Huntington's disease. Brain Pathol 20, 96-105, doi:10.1111/j.1750-3639.2008.00249.x (2010).
56 Conforti, P. et al. In vivo delivery of DN:REST improves transcriptional changes of REST-regulated genes in HD mice. Gene Ther 20, 678-685, doi:10.1038/gt.2012.84 (2013).
57 Jodeiri Farshbaf, M. & Ghaedi, K. Huntington's Disease and Mitochondria. Neurotox Res 32, 518-529, doi:10.1007/s12640-017-9766-1 (2017).
58 Naia, L., Ferreira, I. L., Ferreiro, E. & Rego, A. C. Mitochondrial Ca(2+) handling in Huntington's and Alzheimer's diseases—Role of ER-mitochondria crosstalk. Biochem Biophys Res Commun 483, 1069-1077, doi:10.1016/j.bbrc.2016.07.122 (2017).
59 Zheng, J., Winderickx, J., Franssens, V. & Liu, B. A Mitochondria-Associated Oxidative Stress Perspective on Huntington's Disease. Front Mol Neurosci 11, 329, doi:10.3389/fnmol.2018.00329 (2018).
60 Cherubini, M., Lopez-Molina, L. & Gines, S. Mitochondrial fission in Huntington's disease mouse striatum disrupts ER-mitochondria contacts leading to disturbances in Ca(2+) efflux and Reactive Oxygen Species (ROS) homeostasis. Neurobiol Dis 136, 104741, doi:10.1016/j.nbd.2020.104741 (2020).
61 Strand, A. D. et al. Expression profiling of Huntington's disease models suggests that brain-derived neurotrophic factor depletion plays a major role in striatal degeneration. J Neurosci 27, 11758-11768, doi:10.1523/JNEUROSCI.2461-07.2007 (2007).
62 Chiang, M. C., Chern, Y. & Huang, R. N. PPARgamma rescue of the mitochondrial dysfunction in Huntington's disease. Neurobiol Dis 45, 322-328, doi:10.1016/j.nbd.2011.08.016 (2012).
63 Puigserver, P. et al. A cold-inducible coactivator of nuclear receptors linked to adaptive thermogenesis. Cell 92, 829-839, doi:10.1016/s0092-8674(00)81410-5 (1998).
64 Narain, Y., Wyttenbach, A., Rankin, J., Furlong, R. A. & Rubinsztein, D. C. A molecular investigation of true dominance in Huntington's disease. J Med Genet 36, 739-746, doi:10.1136/jmg.36.10.739 (1999).
65 Arai, T. et al. TDP-43 is a component of ubiquitin-positive tau-negative inclusions in frontotemporal lobar degeneration and amyotrophic lateral sclerosis. Biochem Biophys Res Commun 351, 602-611, doi:10.1016/j.bbrc.2006.10.093 (2006).
66 Neumann, M. et al. Ubiquitinated TDP-43 in frontotemporal lobar degeneration and amyotrophic lateral sclerosis. Science 314, 130-133, doi:10.1126/science.1134108 (2006).
67 Elden, A. C. et al. Ataxin-2 intermediate-length polyglutamine expansions are associated with increased risk for ALS. Nature 466, 1069-1075, doi:10.1038/nature09320 (2010).
68 Cruts, M. et al. Null mutations in progranulin cause ubiquitin-positive frontotemporal dementia linked to chromosome 17q21. Nature 442, 920-924, doi:10.1038/nature05017 (2006).
69 Tanaka, Y., Matsuwaki, T., Yamanouchi, K. & Nishihara, M. Increased lysosomal biogenesis in activated microglia and exacerbated neuronal damage after traumatic brain injury in progranulin-deficient mice. Neuroscience 250, 8-19, doi:10.1016/j.neuroscience.2013.06.049 (2013).
70 Wu, Y. et al. Multi-trait analysis for genome-wide association study of five psychiatric disorders. Transl Psychiatry 10, 209, doi:10.1038/s41398-020-00902-6 (2020).
71 Ni, H. et al. The GWAS Risk Genes for Depression May Be Actively Involved in Alzheimer's Disease. J Alzheimers Dis 64, 1149-1161, doi:10.3233/JAD-180276 (2018).
72 Finnema, S. J. et al. Imaging synaptic density in the living human brain. Sci Transl Med 8, 348ra396, doi:10.1126/scitranslmed.aaf6667 (2016).
73 Synthetic Peptides: A User's Guide (Advances in Molecular Biology), Grant G. A. ed., Oxford University Press, 2002
74 Pharmaceutical Formulation: Development of Peptides and Proteins, Frokjaer and Hovgaard eds., Taylor and Francis, 1999
75 Remington's Pharmaceutical Sciences, 17th ed., Mack Publishing Company, Easton, P A, 1985, p. 1418

1. A cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a pharmaceutically acceptable salt thereof.

2. The cyclic peptide according to claim 1, wherein the cyclic peptide is backbone cyclized.

3. The cyclic peptide or pharmaceutically acceptable salt according to either of claim 1 or 2, wherein the cyclic peptide comprises no more than 50 amino acid residues, such as no more than 40 amino acid residues, such as no more than 30 amino acid residues, such as no more than 20 amino acid residues.

4. The cyclic peptide or pharmaceutically acceptable salt according to claim 3, wherein the cyclic peptide comprises no more than 14 amino acid residues, such as no more than 13 amino acid residues, such as no more than 12 amino acid residues.

5. The cyclic peptide or pharmaceutically acceptable salt according to any one of claims 1 to 4, wherein the cyclic peptide comprises the amino acid sequence of MTEPVEHEEDV (SEQ ID NO: 1).

6. The cyclic peptide or pharmaceutically acceptable salt according to claim 5, wherein the cyclic peptide consists of the amino acid sequence of MTEPVEHEEDV (SEQ ID NO: 1).

7. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MTEPVEHEEDV (SEQ ID NO: 1).

8. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MTDPVDHDEDV (SEQ ID NO: 2).

9. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MTAPVAHAEDV (SEQ ID NO: 3).

10. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MIEPVEHEESR (SEQ ID NO: 4).

11. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MIDPVDHDESR (SEQ ID NO: 5).

12. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MIGSVEQEENA (SEQ ID NO: 6).

13. The cyclic peptide or pharmaceutically acceptable salt according to claim 1, wherein the peptide is backbone cyclized, all residues of the peptide are joined exclusively by peptide bonds, the peptide is unmodified and consists of the amino acid sequence of MIGSVDQDENA (SEQ ID NO: 7).

14. A pharmaceutical composition comprising the peptide or pharmaceutically acceptable salt according to any one of claims 1 to 13.

15. The cyclic peptide or pharmaceutically acceptable salt according to any one of claims 1 to 13 or the pharmaceutical composition according to claim 14 for use as a medicament.

16. The cyclic peptide or pharmaceutically acceptable salt according to any one of claims 1 to 13 or the pharmaceutical composition according to claim 14 for use in the treatment or prevention of a disease or disorder selected from the group consisting of diseases of the nervous system; neuropathic pain; mental and behavioural disorders; stroke, metabolic disorders and WAGR syndrome.

17. The cyclic peptide, pharmaceutically acceptable salt or pharmaceutical composition for use according to claim 16, wherein the diseases of the nervous system is selected from the group consisting of Huntington's disease, amyotrophic lateral sclerosis (ALS), Parkinson's disease, Alzheimer's disease, Frontotemporal dementia (FTD) and epilepsy.

18. The cyclic peptide, pharmaceutically acceptable salt or pharmaceutical composition for use according to claim 16, wherein the diseases of the nervous system is a neurodegenerative disease.

19. The cyclic peptide, pharmaceutically acceptable salt or pharmaceutical composition for use according to claim 18, wherein the neurodegenerative disease is selected from the group consisting of Frontotemporal dementia (FTD), Huntington's disease, Alzheimer's disease, Parkinson's disease and amyotrophic lateral sclerosis.

20. The cyclic peptide, pharmaceutically acceptable salt or pharmaceutical composition for use according to claim 16, wherein the mental and behavioural disorder is selected from the group consisting of dementia, depression, anxiety, post-traumatic stress disorder (PTSD), Schizophrenia (SZ), attention deficit hyperactivity disorder (ADHD), autism, Rett syndrome, Fragile X syndrome and Angelman syndrome.

21. The cyclic peptide, pharmaceutically acceptable salt or pharmaceutical composition for use according to claim 16, wherein the metabolic disorder is selected from the group consisting of obesity; diabetes mellitus type 1; diabetes mellitus type 2, non-alcoholic fatty liver disease (NAFLD) and lysosomal storage disorders, such as Nieman-Pick disease.

22. A method of treatment or prevention of a disease or disorder selected from the group consisting of diseases of the nervous system; neuropathic pain; mental and behavioural disorders; stroke; metabolic disorders and WAGR syndrome, said method comprising administering the cyclic peptide according to any one of claims 1 to 13 or the pharmaceutical composition according to claim 14.

23. Use of the cyclic peptide or pharmaceutically acceptable salt according to any one of claims 1 to 13 or the pharmaceutical composition according to claim 14 for the manufacture of a medicament for the treatment or prevention of a disease or disorder selected from the group consisting of diseases of the nervous system; neuropathic pain; mental and behavioural disorders; stroke; metabolic disorders and WAGR syndrome.

24. A method of manufacturing a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7), or a salt thereof, the method comprising the steps of:
(i) Preparing a linear peptide or a salt thereof, or a protected version thereof, having an appropriate amino acid sequence; and
(ii) subsequently generating a cyclized peptide, or a salt thereof, from the linear peptide, salt thereof, or a protected version thereof.

25. A linear amino acid comprising a sequence selected from the group consisting of SEQ ID NO: 10 to 16 and 20 to 89, a salt thereof, or a protected version thereof.

26. The linear amino acid according to claim 25, consisting of a sequence selected from the group consisting of SEQ ID NO: 10 to 16 and 20 to 89, a salt thereof, or a protected version thereof.

27. A salt of a cyclic peptide comprising an amino acid sequence selected from the group consisting of MTEPVEHEEDV (SEQ ID NO: 1), MTDPVDHDEDV (SEQ ID NO: 2), MTAPVAHAEDV (SEQ ID NO: 3), MIEPVEHEESR (SEQ ID NO: 4), MIDPVDHDESR (SEQ ID NO: 5), MIGSVEQEENA (SEQ ID NO: 6) and MIGSVDQDENA (SEQ ID NO: 7).

28. A nucleic acid construct encoding for a peptide comprising an amino acid sequence selected from the group consisting of SEQ ID NO: 10 to 16 and 20 to 89.

29. The nucleic acid construct encoding for a peptide consisting of an amino acid sequence selected from the group consisting of SEQ ID NO: 10 to 16 and 20 to 89.

30. A vector comprising the nucleic acid construct according to either claim 28 or 29.

31. An isolated host cell comprising the nucleic acid construct according to claim 28 or 29, or a vector according to claim 30.


SEQUENCE LISTING (90 sequences)

SEQ ID NO: 1 - protein, 11 aa, Artificial Sequence
  Cyclic peptide
MTEPVEHEEDV

SEQ ID NO: 2 - protein, 11 aa, Artificial Sequence
  Cyclic peptide
MTDPVDHDEDV

SEQ ID NO: 3 - protein, 11 aa, Artificial Sequence
  Cyclic peptide
MTAPVAHAEDV

SEQ ID NO: 4 - protein, 11 aa, Artificial Sequence
  Cyclic peptide
MIEPVEHEESR

SEQ ID NO: 5 - protein, 11 aa, Artificial Sequence
  Cyclic peptide
MIDPVDHDESR

SEQ ID NO: 6 - protein, 11 aa, Artificial Sequence
  Cyclic peptide
MIGSVEQEENA

SEQ ID NO: 7 - protein, 11 aa, Artificial Sequence
  Cyclic peptide
MIGSVDQDENA

SEQ ID NO: 8 - protein, 11 aa, Artificial Sequence
  Linear, amidated and acetylated peptide
  MOD_RES (1)..(1): ACETYLATION
  MOD_RES (11)..(11): AMIDATION
MTEPVEHEEDV

SEQ ID NO: 9 - protein, 32 aa, Artificial Sequence
  Linear peptide
YARAAARNARAEKEQEMTDPVDHDEDVQGAVQ

SEQ ID NO: 10 - protein, 11 aa, Artificial Sequence
  Peptide
MTEPVEHEEDV

SEQ ID NO: 11 - protein, 11 aa, Artificial Sequence
  Peptide
MTDPVDHDEDV

SEQ ID NO: 12 - protein, 11 aa, Artificial Sequence
  Peptide
MTAPVAHAEDV

SEQ ID NO: 13 - protein, 11 aa, Artificial Sequence
  Peptide
MIEPVEHEESR

SEQ ID NO: 14 - protein, 11 aa, Artificial Sequence
  Peptide
MIDPVDHDESR

SEQ ID NO: 15 - protein, 11 aa, Artificial Sequence
  Peptide
MIGSVEQEENA

SEQ ID NO: 16 - protein, 11 aa, Artificial Sequence
  Peptide
MIGSVDQDENA

SEQ ID NO: 17 - protein, 11 aa, Homo sapiens
MTSPVSHSEDV

SEQ ID NO: 18 - protein, 11 aa, Homo sapiens
MISPVSHSESR

SEQ ID NO: 19 - protein, 11 aa, Homo sapiens
MIGSVSQSENA

SEQ ID NO: 20 - protein, 11 aa, Artificial Sequence
  Peptide
VMTEPVEHEED

SEQ ID NO: 21 - protein, 11 aa, Artificial Sequence
  Peptide
DVMTEPVEHEE

SEQ ID NO: 22 - protein, 11 aa, Artificial Sequence
  Peptide
EDVMTEPVEHE

SEQ ID NO: 23 - protein, 11 aa, Artificial Sequence
  Peptide
EEDVMTEPVEH

SEQ ID NO: 24 - protein, 11 aa, Artificial Sequence
  Peptide
HEEDVMTEPVE

SEQ ID NO: 25 - protein, 11 aa, Artificial Sequence
  Peptide
EHEEDVMTEPV

SEQ ID NO: 26 - protein, 11 aa, Artificial Sequence
  Peptide
VEHEEDVMTEP

SEQ ID NO: 27 - protein, 11 aa, Artificial Sequence
  Peptide
PVEHEEDVMTE

SEQ ID NO: 28 - protein, 11 aa, Artificial Sequence
  Peptide
EPVEHEEDVMT

SEQ ID NO: 29 - protein, 11 aa, Artificial Sequence
  Peptide
TEPVEHEEDVM

SEQ ID NO: 30 - protein, 11 aa, Artificial Sequence
  Peptide
VMTDPVDHDED

SEQ ID NO: 31 - protein, 11 aa, Artificial Sequence
  Peptide
DVMTDPVDHDE

SEQ ID NO: 32 - protein, 11 aa, Artificial Sequence
  Peptide
EDVMTDPVDHD

SEQ ID NO: 33 - protein, 11 aa, Artificial Sequence
  Peptide
DEDVMTDPVDH

SEQ ID NO: 34 - protein, 11 aa, Artificial Sequence
  Peptide
HDEDVMTDPVD

SEQ ID NO: 35 - protein, 11 aa, Artificial Sequence
  Peptide
DHDEDVMTDPV

SEQ ID NO: 36 - protein, 11 aa, Artificial Sequence
  Peptide
VDHDEDVMTDP

SEQ ID NO: 37 - protein, 11 aa, Artificial Sequence
  Peptide
PVDHDEDVMTD

SEQ ID NO: 38 - protein, 11 aa, Artificial Sequence
  Peptide
DPVDHDEDVMT

SEQ ID NO: 39 - protein, 11 aa, Artificial Sequence
  Peptide
TDPVDHDEDVM

SEQ ID NO: 40 - protein, 11 aa, Artificial Sequence
  Peptide
VMTAPVAHAED

SEQ ID NO: 41 - protein, 11 aa, Artificial Sequence
  Peptide
DVMTAPVAHAE

SEQ ID NO: 42 - protein, 11 aa, Artificial Sequence
  Peptide
EDVMTAPVAHA

SEQ ID NO: 43 - protein, 11 aa, Artificial Sequence
  Peptide
AEDVMTAPVAH

SEQ ID NO: 44 - protein, 11 aa, Artificial Sequence
  Peptide
HAEDVMTAPVA

SEQ ID NO: 45 - protein, 11 aa, Artificial Sequence
  Peptide
AHAEDVMTAPV

SEQ ID NO: 46 - protein, 11 aa, Artificial Sequence
  Peptide
VAHAEDVMTAP

SEQ ID NO: 47 - protein, 11 aa, Artificial Sequence
  Peptide
PVAHAEDVMTA

SEQ ID NO: 48 - protein, 11 aa, Artificial Sequence
  Peptide
APVAHAEDVMT

SEQ ID NO: 49 - protein, 11 aa, Artificial Sequence
  Peptide
TAPVAHAEDVM

SEQ ID NO: 50 - protein, 11 aa, Artificial Sequence
  Peptide
RMIEPVEHEES

SEQ ID NO: 51 - protein, 11 aa, Artificial Sequence
  Peptide
SRMIEPVEHEE

SEQ ID NO: 52 - protein, 11 aa, Artificial Sequence
  Peptide
ESRMIEPVEHE

SEQ ID NO: 53 - protein, 11 aa, Artificial Sequence
  Peptide
EESRMIEPVEH

SEQ ID NO: 54 - protein, 11 aa, Artificial Sequence
  Peptide
HEESRMIEPVE

SEQ ID NO: 55 - protein, 11 aa, Artificial Sequence
  Peptide
EHEESRMIEPV

SEQ ID NO: 56 - protein, 11 aa, Artificial Sequence
  Peptide
VEHEESRMIEP

SEQ ID NO: 57 - protein, 11 aa, Artificial Sequence
  Peptide
PVEHEESRMIE

SEQ ID NO: 58 - protein, 11 aa, Artificial Sequence
  Peptide
EPVEHEESRMI

SEQ ID NO: 59 - protein, 11 aa, Artificial Sequence
  Peptide
IEPVEHEESRM

SEQ ID NO: 60 - protein, 11 aa, Artificial Sequence
  Peptide
RMIDPVDHDES

SEQ ID NO: 61 - protein, 11 aa, Artificial Sequence
  Peptide
SRMIDPVDHDE

SEQ ID NO: 62 - protein, 11 aa, Artificial Sequence
  Peptide
ESRMIDPVDHD

SEQ ID NO: 63 - protein, 11 aa, Artificial Sequence
  Peptide
DESRMIDPVDH

SEQ ID NO: 64 - protein, 11 aa, Artificial Sequence
  Peptide
HDESRMIDPVD

SEQ ID NO: 65 - protein, 11 aa, Artificial Sequence
  Peptide
DHDESRMIDPV

SEQ ID NO: 66 - protein, 11 aa, Artificial Sequence
  Peptide
VDHDESRMIDP

SEQ ID NO: 67 - protein, 11 aa, Artificial Sequence
  Peptide
PVDHDESRMID

SEQ ID NO: 68 - protein, 11 aa, Artificial Sequence
  Peptide
DPVDHDESRMI

SEQ ID NO: 69 - protein, 11 aa, Artificial Sequence
  Peptide
IDPVDHDESRM

SEQ ID NO: 70 - protein, 11 aa, Artificial Sequence
  Peptide
AMIGSVEQEEN

SEQ ID NO: 71 - protein, 11 aa, Artificial Sequence
  Peptide
NAMIGSVEQEE

SEQ ID NO: 72 - protein, 11 aa, Artificial Sequence
  Peptide
ENAMIGSVEQE

SEQ ID NO: 73 - protein, 11 aa, Artificial Sequence
  Peptide
EENAMIGSVEQ

SEQ ID NO: 74 - protein, 11 aa, Artificial Sequence
  Peptide
QEENAMIGSVE

SEQ ID NO: 75 - protein, 11 aa, Artificial Sequence
  Peptide
EQEENAMIGSV

SEQ ID NO: 76 - protein, 11 aa, Artificial Sequence
  Peptide
VEQEENAMIGS

SEQ ID NO: 77 - protein, 11 aa, Artificial Sequence
  Peptide
SVEQEENAMIG

SEQ ID NO: 78 - protein, 11 aa, Artificial Sequence
  Peptide
GSVEQEENAMI

SEQ ID NO: 79 - protein, 11 aa, Artificial Sequence
  Peptide
IGSVEQEENAM

SEQ ID NO: 80 - protein, 11 aa, Artificial Sequence
  Peptide
AMIGSVDQDEN

SEQ ID NO: 81 - protein, 11 aa, Artificial Sequence
  Peptide
NAMIGSVDQDE

SEQ ID NO: 82 - protein, 11 aa, Artificial Sequence
  Peptide
ENAMIGSVDQD

SEQ ID NO: 83 - protein, 11 aa, Artificial Sequence
  Peptide
DENAMIGSVDQ

SEQ ID NO: 84 - protein, 11 aa, Artificial Sequence
  Peptide
QDENAMIGSVD

SEQ ID NO: 85 - protein, 11 aa, Artificial Sequence
  Peptide
DQDENAMIGSV

SEQ ID NO: 86 - protein, 11 aa, Artificial Sequence
  Peptide
VDQDENAMIGS

SEQ ID NO: 87 - protein, 11 aa, Artificial Sequence
  Peptide
SVDQDENAMIG

SEQ ID NO: 88 - protein, 11 aa, Artificial Sequence
  Peptide
GSVDQDENAMI

SEQ ID NO: 89 - protein, 11 aa, Artificial Sequence
  Peptide
IGSVDQDENAM

SEQ ID NO: 90 - protein, 56 aa, Artificial Sequence
  Scrambled peptide
YARAAARNARAMDRTVQKVFEHQENYRIYVKRGDPATEAQDKFALAKGHEGVQPDM